(19) United States
(12) Patent Application Publication
(10) Pub. No.: US 2016/0263247 A1
(43) Pub. Date: Sep. 15, 2016

(54) COMPOSITIONS AND METHODS FOR TREATING CARDIOVASCULAR DISEASES USING FOXP3

(71) Applicant: Kiromic, LLC, Lubbock, TX (US)

(72) Inventor: Maurizio Chiriva-Internati, Lubbock, TX (US)

(21) Appl. No.: 15/053,171

(22) Filed: Feb. 25, 2016

Related U.S. Application Data
(63) Continuation-in-part of application No. 14/687,804, filed on Apr. 15, 2015.
Application No. 14/687,804 is a continuation-in-part of application No. 14/508,642, filed on Oct. 7, 2014.
(60) Provisional application No. 61/979,789, filed on Apr. 15, 2014.
(60) Provisional application No. 61/887,704, filed on Oct. 7, 2013.
(60) Provisional application No. 62/120,442, filed on Feb. 25, 2015.

(51) Int. Cl. A61K 48/00; C12N 7/00

(52) U.S. Cl. CPC A61K 48/0058; C12N 7/00; C12N 2710/16171; C12N 2710/16143; C12N 2750/14171; C12N 2750/14143

(57) ABSTRACT

The methods and systems of the present invention provide for an expression vector containing a disease-specific promoter or a constitutive promoter linked to a gene encoding a therapeutic agent, such as a protein, microRNA, siRNA or other therapeutical molecule, e.g., other oligonucletide. The therapeutic agent may be, for example, Interleukin 10 (IL10), Forkhead box P3 (FOXP3), or a member of a transforming growth factor beta 1 (TGFβ1) signaling pathway, such as SMAD3.

### CROSS REFERENCE TO RELATED APPLICATION

This application claims the benefit of U.S. Provisional Patent Application No. 62/120,442 filed on Feb. 25, 2015. This application also claims priority to U.S. patent application Ser. No. 14/687,804, filed on Apr. 15, 2015. U.S. patent application Ser. No. 14/687,804 claims priority to U.S. patent application Ser. No. 14/508,642, filed on Oct. 7, 2014, which claims priority to U.S. Provisional Application No. 61/887,704 filed on Oct. 7, 2013. U.S. patent application Ser. No. 14/687,804 also claims the benefit of U.S. Provisional Patent Application No. 61/979,789 filed on Apr. 15, 2014. All applications are incorporated herein by reference in their entirety.

### GOVERNMENT LICENSE RIGHTS

This invention was made with government support under Merit Review grant awarded by the U.S. Department of Veterans Affairs. The government may have certain rights in the invention.

### FIELD OF THE INVENTION

The present invention relates to methods and compositions for the treatment of cardiovascular diseases and other conditions. In particular, the present invention relates to gene therapy using a disease-specific promoter or a constitutive promoter in treating cardiovascular diseases, and to modulating immune responses, involving FOXP3 (forkhead box P3).

### BACKGROUND OF THE INVENTION

In gene therapy, a therapeutic gene is delivered which then acts to counteract a negative phenotype or disease within the patient or animal model. There are two primary types of gene expression approaches that may be used to drive gene expression: (i) constitutive, and (ii) tissue specific. The gene therapy agents must be safe, and not induce wide-spread unintended damages. However, many, if not all, therapeutic genes will likely have negative consequences when expressed, i.e., adverse reactions, especially if the genes are expressed at high levels.

The first major expression approach is the “constitutive” approach, such as using the cytomegalovirus (CMV) immediate early promoter (“pr”). The treatment of genetic diseases might be the most appropriate for this approach. Genetic diseases result from a faulty protein which is important within a tissue or organ for normal function. Thus, the strategy is usually for maximum gene delivery and gene expression.

However, many, if not most, therapeutic genes may also produce side effects because of their inherent function and adverse reactions when expressed at high levels. For example, in the case of inflammatory diseases, such as cardiovascular diseases, therapeutic genes that have been used in the treatment of atherosclerosis, such as, interleukin 10 (IL10, IL-10), is also associated with a number of adverse reactions which are manifested in the clinical setting and in some animal models. These adverse reactions include increased bacterial, fungal and viral infections, cancer, headache, and anemia (8-16). In general, the strongest therapeutic genes can also be the most dangerous and need be tightly regulated. Some therapeutic genes, however, may be appropriately expressed constitutively in a patient.

A major refinement of the constitutive approach is the “tissue-specific” approach. Here, the delivered transgene is expressed only within a specific cell type(s), thereby limiting its overall expression (17). This approach may represent an improvement over the constitutive approach.

The third approach is a “disease-specific” approach (4). The goal of the disease-specific approach is to limit the expression of the therapeutic transgene to the site of disease, in the cells which are changing towards the disease-associated phenotype. This disease-specific approach provides an important safety feature for gene therapy against adverse reactions from the over-expression of a therapeutic transgenes such as IL10.

Because of the potential advantages of the disease-specific approach, there is a continuing need to develop and test new expression systems. Additionally, there is a need to identify additional therapeutic genes that may have similar or better therapeutic effect when comparied to other therapeutic genes, which may be associated with fewer adverse effects even when constitutively expressed.

### SUMMARY

The present invention provides methods of treating a cardiovascular disease in a subject comprising the step of administering to the subject a vector comprising a cDNA encoding a FOX (Forkhead box) protein. The FOX protein may be FOXP3, such as human FOXP3. The vector may be under the control of a disease-specific promoter, a tissue-specific promoter, or a constitutive promoter.

The cardiovascular disease may be, for example, atherosclerosis, coronary artery disease, or hypertension.

In certain embodiments, the promoter is a disease-specific promoter. The promoter may be a lectin-like oxidized low density lipoprotein receptor 1 (LOX-1) promoter. In one embodiment, the promoter comprising a nucleotide sequence about 80% to about 100% identical to the nucleotide sequence of SEQ ID NO: 1.

In certain embodiments, the promoter is a constitutive promoter. The promoter may be a cytomegalovirus (CMV) immediate early promoter.

The expression vector may be an adeno-associated virus (AAV) vector (derived from AAV). For example, the AAV vector may be an AAV2 or AAV8 vector. The AAV vector may comprise an AAV8 capsid gene which can be wild-type or mutated. In one embodiment, the AAV8 capsid gene of the AAV vector comprises SEQ ID NO: 3; SEQ ID NO: 5; or (iii) SEQ ID NO: 7.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1: Structure of AAV vectors and experimental scheme. A. is a cartoon showing the basic structure of the three AAV vectors used in this study. B. shows the experimental scheme and data collected.

FIG. 2: More detailed structure of the AAV.LOX1pr-hIL10 vector plasmid. This figure shows in more detail the structure of the basic structure of the AAV2.LOX1pr-hIL10 vector plasmid used to generate the virus. Note that the relative positions of three responsive elements for AngII, Ox-LDL and PMA are shown within the promoter sequence. However, the full length LOX1pr is likely responsive to many other stimuli as well.

FIG. 3: Physical characterization of animal groups. Data shown are mean+/−SE. A. shows the levels of total cholesterol. Note that cholesterol levels of all the animals on HCD had had significantly higher cholesterol levels than ND animals. B. shows the animal weights at the end of the experiment. The key at the bottom is used for both panels.

FIG. 4: Expression of hIL10 in animal groups. Relative expression of delivered hIL10 genes compared to endogenous β-actin determined by real-time quantitative PCR from aorta of 6 mice in each group. For QRT-PCR the quantity of mRNA for each vector transgene was normalized to βactin in the same sample. Data shown are mean+/−SE. Note that while CMVpr-IL10 (CMV promoter-IL10) and LOX1pr-IL10 treatments were statistically similar, however LOX1pr-IL10 trended to be less than half the expression level of CMVpr-IL-10. Disease-specific Expression of hIL10 in various organs: LOX-1 promoter is predominantly expressed in the aorta, the site of disease (57).

FIG. 5: Systolic blood velocity. High resolution ultrasound (HRUS) was used to measure blood flow velocities in the luminal center of the abdominal region of the aorta in 8-10 animals from each group. Shown is a quantification of the results. Note that both CMVpr-hIL10- and LOX1pr-hIL10-HCD-treated animals all had significantly lower blood velocity than the AAV/Neo-HCD-treated animals, similar to ND controls. However, CMVpr-hIL10-HCD- and LOX1pr-hIL10-HCD-treated animals were statistically similar to each other.

FIG. 6: Analysis of the aortic lumen by high resolution ultrasound (HRUS). HRUS was used to measure the cross sectional area of the thoracic region of the aortas in 8-10 animals from each animal group. Shown is a quantification of the cross-sectional area for the abdominal/thoracic region of the aorta. Note that both CMVpr-hIL10- and LOX1pr-hIL10-HCD-treated animals had a significantly larger cross sectional area than the AAV/Neo-HCD-treated animals, indicating significant efficacy. However, CMVpr-hIL10-HCD- and LOX1pr-hIL10-HCD-treated animals were statistically similar to each other.

FIG. 7: Analysis of the aortic wall thickness by HRUS. HRUS was used to measure the wall thickness of the aorta. Shown is a quantification of the thoracic region of the aortas in 8-10 animals from each animal group. Note that both CMVpr-hIL10- and LOX1pr-hIL10-HCD-treated animals had a significantly thinner aortic wall than the AAV/Neo-HCD-treated animals, indicating significant efficacy. However, CMVpr-hIL10-HCD- and LOX1pr-hIL10-HCD-treated animals were statistically similar to each other.

FIG. 8. Transduction of human primary placental umbilical cord vein rings in culture by AAV2 and modified AAV2-EYH. Vein rings were infected with 10⁸ AAV2-CMV-EYH/eGFP virus in vitro. Photomicrographs were taken 2 days post-infection. Note that the smooth muscle cells are strongly targeted by the AAV2-EYH-modified capsid.

FIG. 9. Specific changes in the AAV8 capsid for improved transduction of arterial smooth muscle cells, including tasks 1 and 2. The indicated changes will be made in pDG8). Tyrosines at position 447 and 733 will be replaced by phenylalanines, while the EYH peptide (a peptide having seven amino acids), will be inserted after position 590. These modifications may enhance delivery of the AAV2/8(cvd1). LOX1pr-IL10 DNA into vascular smooth muscle cells.

FIG. 10. Delivery of hSMAD3 and dietary effects. A. Relative expression of hSMAD3 gene to βactin by real-time quantitative PCR from aorta of 3 mice in each group. For qRT-PCR the quantity of RNA for each gene was normalized to βactin in the same sample. Data shown are mean+/−SE. B. Western blot analysis of protein from liver probed with anti-SMAD3 antibody. Note that both A and B show increased SMAD3 levels in the AAV/hSMAD3-treated animals. C. Levels of total cholesterol. (HCD: High Cholesterol Diet).

FIG. 11. Structural Analysis of the aorta. High resolution ultrasound (HRUS) was used to measure various aortic parameters. A. Quantification of the cross-sectional area for the thoracic region of the aorta in 3-5 animals from each animal group by HRUS with representative captured images from the analysis just above. Note that the AAV/hSMAD3-HCD animals had a larger cross sectional area than the AAV/Neo-HCD animals. B. Quantification of the wall thickness of the aorta (thoracic region). Note that the AAV/hSMAD3-HCD animals have a thinner wall thickness than the AAV/Neo-HCD animals. C. Quantification of blood flow velocities in the lumenal center of the abdominal region of the aorta in 3-5 animals from each group with representative captured images from the analysis just above. Note that the AAV/hSMAD3-HCD animals have a much lower blood velocity than the AAV/Neo-HCD animals (or hCGRP-treated).

FIG. 12. Macrophage burden of aortic tissue. A. CD68 expression. CD68 is a marker of macrophages and thus is a general marker of inflammation. Histologic sections of aorta from the indicated animal groups were analyzed for CD68 protein by immunohistochemistry using anti-CD68 antibody. Note that the AAV/hSMAD3-HCD-treated animals displayed a much lower brown CD68 signal than the AAV/Neo-HCD-treated animals strongly suggesting lower inflammation. B. Similar analysis with anti-ITGAM antibody, another marker of macrophages, with similar results to CD68. C. QRT-PCR analysis of EMR expression, another macrophage marker. Note, again, macrophage levels were significantly lower (p<0.05) in hSMAD3-treated animals than Neo-treated. D. QRT-PCR analysis of ITGAM expression. Note, again, macrophage levels trended lower in hSMAD3-treated animals than Neo-treated.

FIG. 13. Visual inspection of representative aortas. Aortas from the indicated animals were buffered formalin fixed, cleaned and photographed. Note that the AAV/Neo-treated HCD aorta displays much higher amounts of lipid accumulation (white areas) than the AAV/hSMAD3-treated-HCD animals.

FIG. 14. Immune response status of Th2 in the aortas. A. QRT-PCR analysis of IL-4 expression, a Th2 response cytokine. Note that IL-4 levels were significantly higher (p <0.05) in hSMAD3-treated animals than Neo-treated. B. QRT-PCR analysis of IL-10 expression, another Th2 response cytokine. Note, again, IL-10 levels trended higher in hSMAD3-treated animals than Neo-treated. C. QRT-PCR analysis of IL-7 expression, a Th1 response cytokine. Note that IL-7 levels were higher (p<0.05) in hSMAD3-treated animals than Neo-treated, however, overall, the changes were very minor. D. QRT-PCR analysis of IL-12 expression, another Th1 response cytokine. Note IL-12 levels trended lower in hSMAD3-treated animals than Neo-treated.

FIG. 15. Collagen (COL) and connective tissue growth factor (CTGF) expression in aortas and liver. A. QRT-PCR analysis of COL1A2 expression in the aortas, a major marker of fibrosis. Note that COL1A2 levels were essentially the same in the aortas of both hSMAD3-treated animals and Neo-treated. B. QRT-PCR analysis of COL2A1 expression in the aortas, another marker of fibrosis. Note that COL2Al2 levels were significantly lower in the aortas of both hSMAD3-treated animals than Neo-treated. C. QRT-PCR analysis of COL1A2 expression in the liver. Note that COL1A2 levels trended lower in both hSMAD3-treated animals than Neo-treated. No significant change is seen in any experimental group. D, E, and F show an analogous Q-RT-PCR analysis of COL1A1, COL2A1, and CTGF expression, respectively, but this time in the liver. Note that all three genes are significantly down-regulated by hSMAD3 delivery compared to Neo control, fully consistent with lower fibrosis.

FIG. 16 Vector structure and experimental overview for delivery of FOXP3. A. The structure of the AAV virus vectors for delivery of FOXP3. B. The experimental overview for delivery of FOXP3.

FIG. 17. Animal characterization. A. The levels of total cholesterol at week 20. B. The animal weights at the end (week 20) of the experiment. The animals never received fasting conditions. p values of <0.05 refer to the student t test, are indicated by a “asterisk”, and also indicate statistical significance.

FIG. 18. Aortic lumen cross-sectional area measurement. High resolution ultrasound (HRUS) was used to measure the cross-sectional area for the aortas in 6-8 animals from each animal group by HRUS with representative captured images from the analysis shown just above. Note that the AAV/hFOX3P-HCD animals had a larger cross sectional luminal area than the AAV/Neo-HCD animals. Also note that the AAV/hFOX3P-HCD and the ND groups were statistically the same. P values of <0.05 refer to the student t test, are indicated by a “asterisk”, and also indicate statistical significance.

FIG. 19. Aortic wall thickness measurement. HRUS was used to measure the wall thickness of the aorta. Shown is a quantification of the wall thickness of the aorta (thoracic region) of indicated groups with representative captured images from the analysis shown just above. Note that the AAV/hFOXP3-HCD animals have a significantly thinner wall thickness than the AAV/Neo-HCD animals. Also note that the AAV/hFOX3P-HCD and the ND groups were statistically the same. p values of <0.05 refer to the student t test, are indicated by a “asterisk”, and also indicate statistical significance.

FIG. 20. Systolic blood velocity measurement. High resolution ultrasound (HRUS) was used to quantify blood flow velocities in the luminal center of the aorta in 6-8 animals from each group with representative captured images from the analysis shown just above. Note that the AAV/hFOXP3-HCD animals have a much lower blood velocity than the AAV/Neo-HCD animals. Also note that the AAV/hFOX3P-HCD and the ND groups were very similar in peak systolic blood velocity. p values of <0.05 refer to the student t test, are indicated by a “asterisk”, and also indicate statistical significance.

FIG. 21. Visual inspection of representative aortas. A. Aortas from the indicated animals were buffered formalin-fixed, cleaned and photographed. Note that the AAV/Neo-treated HCD aorta displays much higher amounts of lipid accumulation (white areas) than ether the AAV/hFOXP3-HCD-treated animals. B. PowerPoint black-white 25% enhancement of the aortic arch of the three aortas. Note that the AAV/Neo-treated HCD aorta displays a much more extensive white area than ether the AAV/hFOXP3-HCD-treated or ND animals.

FIG. 22. Histologic views of representative aortas. Aortas from each of three representative animals from the three indicated animal groups (indicated column) were buffered formalin-fixed, paraffin-embedded, sectioned and hematoxylin and eosin stained. Note that the higher level of atherosclerotic plaque in the AAV/Neo-HCD-treated group is readily apparent.

### DETAILED DESCRIPTION

The methods and systems of the present invention provide for an expression vector containing a disease-specific promoter, a tissue-specific promoter, or a constitutive promoter linked to a gene encoding a therapeutic agent, such as a protein, microRNA, siRNA or other therapeutic molecule, e.g., other oligonucleotide. A variety of different disease-specific promoters may be used with the present invention, provided that the promoter preferentially expresses the gene linked to it at the site of the disease and not more globally within the body.

Because of the adverse reactions which can arise from the use of certain therapeutic agents, their overall production may be limited. The use of disease-specific transcriptional promoters is one approach to give such reduced and selective expression. Such disease-specific gene expression should direct expression to the site of disease where it is most needed and, at the same time, limit overall production. The disease-specific expression of a therapeutic agent can lower the possibility of significant adverse reactions (17-23).

In another embodiment, the methods and systems of the present invention involves a constitutive promoter.

The present invention provides for an expression vector for treating a variety of conditions including cardiovascular diseases, e.g., atherosclerosis. The expression vector may be an adeno-associated virus (AAV) vector (derived from AAV). In one embodiment, the disease-specific promoter is the promoter of the LOX-1 gene (LOX-1 promoter, LOX1 promoter, or LOX1pr) (17, 4). In another embodiment, the promoter is a cytomegalovirus (CMV) immediate early promoter. In a third embodiment, the promoter is a tissue-specific promoter. The therapeutic agent may be FOXP3, or Interleukin 10 (IL 10).

### Promoters, Including Disease-Specific Promoters

In certain embodiments, the promoter may be a constitutive promoter. For example, the promoter may be largely or constantly active, and may be without specificity for particular cell types. For example, the promoter may be the cytomegalovirus (CMV) immediate early promoter (pr), the simian virus 40 early (SV40) promoter, the human Ubiquitin C (UBC) promoter, the human elongation factor 1α (EF1A) promoter, the human or mouse phosphoglycerate kinase 1 (PGK1) promoter, human beta actin promoter, or the chicken β-Actin promoter coupled with CMV early enhancer (CAGG). Such a promoter may be useful where there is less concern about a therapeutic gene leading to adverse reactions if expressed at high levels in many cell types, and/or where a compatible disease-specific or tissue-specific promoter is unknown or unsuitable.

In certain embodiments, the present expression vector contains a disease-specific promoter. A disease-specific promoter can ensure that the expression of a gene under its control is substantially limited to diseased tissues or tissues surrounding diseased tissues. The promoter of any gene, which is up-regulated during a disease, may be used as a disease-specific promoter in the present invention. Under normal conditions (a non-disease state), the gene, under the control of its promoter, is expressed at a low level which may or may not be detectable. During a disease, the promoter is activated by disease-associated stimuli (e.g., blood sheer stress, dislipidemia, immune cell trafficking, other activations, etc.), and the promoter/gene is strongly up-regulated. The use of a disease-specific promoter also is an inherent safeguard against the adverse effects of a therapeutic agent under its control. Once the disease becomes diminished, the disease-stimulus will be eliminated or reduced (e.g., after the gene therapy). In response, the expression of the therapeutic agent, under the control of the disease-specific promoter, should be down-regulated, resulting in a negative feedback.

Lectin-like oxidized low density lipoprotein receptor 1 (LOX1, LOX-1, oxidized LDL receptor 1, OLR1) is a scavenger receptor which is expressed when cells become activated, and binds many ligands. LOX1 is also one of the receptors which recognize oxidized low density lipoprotein (Ox-LDL). Moreover, it is expressed in many cell types, including endothelial cells, smooth muscle cells, and macrophages, the major cell types believed involved in atherosclerosis (23-28). It is heavily expressed within atherosclerotic plaque. LOX-1 is also considered one of the earliest markers of activated endothelium, which predates the initiation of atherosclerosis (17, 24). LOX1 appears to be transcriptionally up-regulated during atherogenesis. Many agents, such as Ox-LDL and AngII, induce its up-regulation.

In one embodiment of the present invention, the LOX-1 transcriptional promoter (the promoter of the LOX1 gene, LOX1pr) is used as a disease-specific promoter of the expression vector for expressing therapeutic genes to counter a cardiovascular disease (e.g., to counter atherogenesis). The LOX-1 promoter can be from human or from other species (4, 17).

In another embodiment, the present expression vector contains a promoter comprising/consisting of (or consisting essentially of) a nucleotide sequence about 60% to about 100%, about 70% to about 100%, about 80% to about 100%, about 85% to about 100%, about 90% to about 100%, about 95% to about 100%, about 98% to about 100%, about 60%, about 70%, about 80%, about 85%, about 90%, about 95%, about 98%, or about 100% identical to the nucleotide sequence of SEQ ID NO: 1.

Non-limiting examples of the disease-specific promoters also include the promoters of the gene of Apoliporotein E (apoE), superoxide dismutase 2 (SOD2), carcinoembryonic antigen, HER-2/neu, DF3/MUC (Dachs, et al. 1997. Oncol. Res. 9:313-25), tyrosinase, fetoprotein, albumin, CC10, or prostate-specific antigen; tet-responsive element, or Myc-Max response elements. Promoters that may also be used in the present invention include the promoters disclosed in Eyster et al., Gene expression signatures differ with extent of atherosclerosis in monkey iliac artery, Menopause, 2011, 18(10):1087-95. Wilcox et al., Local expression of inflammatory cytokines in human atherosclerotic plaques, J Atheroscler Thromb. 1994; 1 Suppl 1:S10-3. The disclosures of both are incorporated herein by reference in their entirety.

In some embodiments, a tissue-specific promoter may be used, including, but not limited to, myocardium-specific promoters (e.g., cardiac troponin T promoter), smooth muscle cell-specific promoters (e.g., SMCs promoter; SM22a promoter), endothelial cell-specific promoters (e.g., vascular endothelial-cadherin promoter) and macrophage-specific promoters (e.g., scavenger receptor A promoter). The tissue-specific promoters may also include cardiac myosin heavy chain (MHC)-α promoter specific for cardiac tissues (both ventricles and atria), myosin light chain 2 (MLC2 including MLC2v) promoter, the cardiac MHC-2 promoter specific for ventricles, and tie (tie1 or tie2) promoters selective for endothelial cells. Endothelium-specific promoters also include vWF, FLT-1 and ICAM-2 promoters. Fishbein et al., Site-specific gene therapy for cardiovascular disease, Curr. Opin. Drug Discov. Devel. 2010 March; 13(2): 203-213. Toscano, et al., Physiological and tissue-specific vectors for treatment of inherited diseases, Gene Therapy (2011) 18, 117-127.

The promoter may be wild-type or a mutant. Mutants can be created by introducing one or more nucleotide substitutions, additions or deletions. For example, mutations can be introduced by standard techniques, such as site-directed mutagenesis and PCR-mediated mutagenesis. The promoter may be full-length or truncated.

Under the control of the disease-specific promoter, the expression level of a therapeutic gene (encoding a therapeutic agent) in the tissue involved in the condition being treated may be lower than the expression level of the therapeutic gene under the control of a constitutive promoter. In one embodiment, the expression level of a therapeutic gene (encoding, e.g., IL-10) in the blood vessel (e.g., the aorta, an artery, artery wall, etc.) may be about 90%, about 80%, about 70%, about 60%, about 50%, about 40%, about 30%, about 20%, less than about 90%, less than about 80%, less than about 70%, less than about 60%, less than about 50%, less than about 40%, less than about 30%, or less than about 20%, of the expression level of the therapeutic gene under the control of a constitutive promoter (e.g., the cytomegalovirus (CMV) immediate early promoter).

In another embodiment, the expression level of a therapeutic gene (encoding a therapeutic agent) in the tissue not involved in the condition being treated may be lower than the expression level of the therapeutic gene under the control of a constitutive promoter. For example, the expression level of a therapeutic gene (encoding, e.g., IL-10) in the tissue not involved in the condition being treated (e.g., the lungs or liver) may be about 90%, about 80%, about 70%, about 60%, about 50%, about 40%, about 30%, about 20%, about 10%, about 5%, about 2%, about 1%, less than about 90%, less than about 80%, less than about 70%, less than about 60%, less than about 50%, less than about 40%, less than about 30%, less than about 20%, less than about 10%, less than about 5%, less than about 2%, less than about 1%, of the expression level of the therapeutic gene under the control of a constitutive promoter (e.g., the cytomegalovirus (CMV) immediate early promoter).

The level of a protein may be determined by any suitable assays, including, but not limited to, using antibodies specific to the protein in Western blot, enzyme-linked immunosorbent assay (ELISA), flow cytometry, immunocytochemistry or immunohistochemistry.

Gene expression levels may be assayed by any suitable methods, including, but not limited to, measuring mRNA levels and/or protein levels. Levels of mRNA can be quantitatively measured by RT-PCR, Northern blot, next-generation sequencing, etc.

### Therapeutic Genes

The vectors of the present application may further comprise a heterologous gene under the control of the promoter. The gene may be a therapeutic gene encoding a therapeutic agent. As used herein, the phrase “therapeutic agents” refer to any molecules or compounds that assist in the treatment or prevention of a disease.

The therapeutic agent that may be expressed under the control of the disease-specific promoters can be a protein, a polypeptide, a polynucleotide (e.g., a small interfering RNA (siRNA), a microRNA (miRNA), a ribozyme or an antisense molecule), an antibody or antigen-binding portion thereof, or any other molecules. The therapeutic agent may be cytokines, immunoglobulins (e.g., IgG, IgM, IgA, IgD or IgE), integrins, albumin or any other potentially, therapeutically relevant molecule. The choice of the particular molecule is determined by the pathology of a particular disease. Non-limiting examples of the therapeutic agents include Interleukin 1 (IL-1), IL-2, IL-3, IL-4, IL-5, IL-6, IL-7, IL-8, IL-9, IL-10, IL-11 IL-12, GM-CSF and G-CSF.

In one embodiment, therapeutic agents for treating atherosclerosis include agents which down-regulate the immune system, agents which control dyslipidemia (e.g. high cholesterol), and agents which down-regulate anti-reactive oxygen species (anti-ROS). For example, the present therapeutic agents may include transforming growth factor beta 1 (TGFβ1), SMAD3, interleukin 10 (IL-10), STAT3, Netrin-1, angiotensin II type 2 receptor (AT2R), an anti-reactive oxygen species (anti-ROS) agent (e.g., peroxiredoxin 6 (PRDX6) which is an anti-thiol-ROS protein), apolipoprotein Al Milano (ApoA1 milano) which affects dyslipidemia. In one embodiment, the IL-10 gene used in the present vector consists of/comprises the nucleotide sequence of SEQ ID NO: 2.

In another embodiment, the therapeutic agent is a FOX (Forkhead box) protein, including, but not limited to FOXP3, such as human FOXP3.

Human FOXP3 may comprise the amino acid sequence corresponding to GenBank Accession No. AAI43786 (SEQ ID NO: 16), GenBank Accession No. AAI13402 (SEQ ID NO: 17), GenBank Accession No. AAI13404, GenBank Accession No. AAI43787, GenBank Accession No. AAY27088, or GenBank Accession No. ABQ15210. FOXP3 may also comprise other sequences.

The cDNA of human FOXP3 may comprise the nucleotide sequence corresponding to GenBank Accession No. BC143785 (SEQ ID NO: 18), GenBank Accession No. BC113401 (SEQ ID NO: 19), GenBank Accession No. BC113403, GenBank Accession No. BC143786, or GenBank Accession No. EF534714. The cDNA of FOXP3 may also comprise other sequences.

The mRNA of human FOXP3 may comprise the nucleotide sequence corresponding to NCBI Reference Sequence: NM_001114377, or NCBI Reference Sequence: NM_014009. The mRNA of FOXP3 may also comprise other sequences.

In certain embodiments, the present therapeutic agent may comprise/consist of (or consist essentially of) an amino acid sequence about 60% to about 100%, about 70% to about 100%, about 80% to about 100%, about 85% to about 100%, about 90% to about 100%, about 95% to about 100%, about 98% to about 100%, about 60%, about 70%, about 80%, about 85%, about 90%, about 95%, about 98%, or about 100% identical to the amino acid sequence corresponding to GenBank Accession No. AAI43786 (SEQ ID NO: 16), GenBank Accession No. AAI13402 (SEQ ID NO: 17), GenBank Accession No. AAI13404, GenBank Accession No. AAI43787, GenBank Accession No. AAY27088, or GenBank Accession No. ABQ 15210.

In certain embodiments, the present expression vector may contain a therapeutic gene comprising/consisting of (or consisting essentially of) a nucleotide sequence about 60% to about 100%, about 70% to about 100%, about 80% to about 100%, about 85% to about 100%, about 90% to about 100%, about 95% to about 100%, about 98% to about 100%, about 60%, about 70%, about 80%, about 85%, about 90%, about 95%, about 98%, or about 100% identical to the nucleotide sequence of GenBank Accession No. BC143785 (SEQ ID NO: 18), GenBank Accession No. BC113401 (SEQ ID NO: 19), GenBank Accession No. BC113403, GenBank Accession No. BC143786, GenBank Accession No. EF534714, NCBI Reference Sequence: NM_001114377, or NCBI Reference Sequence: NM_014009.

In certain embodiments, the therapeutic agent may be encoded by a gene that is downstream from an alternative therapeutic agent in the alternative agent's signal transduction pathway. For example, the anti-inflammatory abilities of TGFβ1 may work through a number of signal transduction pathways, including Ras-ERK, TAK1-JNK Rho-Rac-cdc42, and mothers against decapentaplegic homologs (SMADs) 2, 3, 4 and others. In certain embodiments, the therapeutic agent is SMAD2, SMAD3, or SMAD4, or any other member of a signal transduction pathway involving TGFβ1. In certain embodiments, the therapeutic agent is SMAD1, SMAD5, SMAD6, SMAD7, or SMAD 8. In certain embodiments, the therapeutic agent is specifically the human homolog of such agents, e.g. hSMAD3.

In certain embodiments, the therapeutic agent may be a member of a TGFβ1 signaling pathway, such as SMAD1, SMAD2, SMAD3, SMAD4, SMAD5, SMAD6, SMAD7, SMAD 8, RhoA, mDia, ROCK, MLC, LIMK, Cofilin, Rac/Cdc42, PAK, c-Abl, Par6, PKC, PI3K, Akt, mTOR, PP2A, p70 S6K, SARA, Shc, GRB2, Smurf1, Smurf2, TAK1/MLK1/MEKK1, MKK3, MKK6, MKK4, p38, JNK, SOS, Ras, Erk1, Erk2, or TMEPAI.

In one embodiment, the therapeutic agent is human SMAD3 whose cDNA corresponds to GenBank Accession No. BC050743. In another embodiment, the therapeutic agent is human SMAD3 whose cDNA corresponds to GenBank Accession No. BC000414.

The present methods and systems (e.g., the expression vectors) may lower the blood (serum or plasma) cholesterol level of a subject by from about 5% to about 90%, from about 10% to about 80%, from about 15% to about 70%, from about 20% to about 60%, from about 10% to about 20%, from about 5% to about 15%, from about 20% to about 30%, from about 30% to about 40%, from about 40% to about 50%, from about 50% to about 60%, from about 60% to about 70%, from about 30% to about 50%, about 10%, about 15%, about 20%, about 30%, about 40%, about 50%, at least about 10%, at least about 15%, at least about 20%, at least about 25%, at least about 30%, at least about 40%, at least about 50%, at least about 60%, or at least about 70%, compared to the blood (serum or plasma) cholesterol level had the present system not been delivered (or compared to the blood (serum or plasma) cholesterol level in a control sample where the present method has not been employed).

The present methods and systems (e.g., the expression vectors) may increase the aortic lumen cross-sectional area of a subject by from about 5% to about 90%, from about 10% to about 80%, from about 15% to about 70%, from about 20% to about 60%, from about 10% to about 20%, from about 5% to about 15%, from about 20% to about 30%, from about 30% to about 40%, from about 40% to about 50%, from about 50% to about 60%, from about 60% to about 70%, from about 30% to about 50%, about 10%, about 15%, about 20%, about 30%, about 40%, about 50%, at least about 10%, at least about 15%, at least about 20%, at least about 25%, at least about 30%, at least about 40%, at least about 50%, at least about 60%, or at least about 70%, compared to the aortic lumen cross-sectional area had the present system not been delivered (or compared to the aortic lumen cross-sectional area in a control subject where the present method has not been employed).

The present methods and systems (e.g., the expression vectors) may decrease the aortic wall thickness of a subject by from about 5% to about 90%, from about 10% to about 80%, from about 15% to about 70%, from about 20% to about 60%, from about 10% to about 20%, from about 5% to about 15%, from about 20% to about 30%, from about 30% to about 40%, from about 40% to about 50%, from about 50% to about 60%, from about 60% to about 70%, from about 30% to about 50%, about 10%, about 15%, about 20%, about 30%, about 40%, about 50%, at least about 10%, at least about 15%, at least about 20%, at least about 25%, at least about 30%, at least about 40%, at least about 50%, at least about 60%, or at least about 70%, compared to the aortic wall thickness had the present system not been delivered (or compared to the aortic wall thickness in a control subject where the present method has not been employed).

The present methods and systems (e.g., the expression vectors) may decrease the systolic blood velocity (or systolic pulse wave velocity) of a subject by from about 5% to about 90%, from about 10% to about 80%, from about 15% to about 70%, from about 20% to about 60%, from about 10% to about 20%, from about 5° to about 15%, from about 20° to about 30%, from about 30° to about 40%, from about 40° to about 50%, from about 50° to about 60%, from about 60° to about 70%, from about 30° to about 50%, about 10%, about 15%, about 20%, about 30%, about 40%, about 50%, at least about 10%, at least about 15%, at least about 20%, at least about 25%, at least about 30%, at least about 40%, at least about 50%, at least about 60%, or at least about 70%, compared to the systolic blood velocity (or systolic pulse wave velocity) had the present system not been delivered (or compared to the systolic blood velocity (or systolic pulse wave velocity) in a control subject where the present method has not been employed).

In one embodiment, the therapeutic agent is a SMAD, including, but not limited to, SMAD1, SMAD2, SMAD3, SMAD4, SMAD5, SMAD6, SMAD7, and SMAD 8. In another embodiment, the therapeutic agent is FOXP3. The therapeutic agent may be any agent described herein.

The present methods and systems (e.g., the expression vectors) may decrease fibrosis. For example, the present methods and systems may decrease the mRNA or protein level of COL2A1 (collagen 2A1) in the aorta of a subject by from about 5° to about 90%, from about 10% to about 80%, from about 15% to about 70%, from about 20% to about 60%, from about 10% to about 20%, from about 5% to about 15%, from about 20° to about 30%, from about 30° to about 40%, from about 20° to about 40%, from about 40° to about 50%, from about 50% to about 60%, from about 60° to about 70%, from about 30% to about 50%, about 10%, about 15%, about 20%, about 30%, about 35%, about 40%, or about 50%, compared to the mRNA or protein level of COL2A1 in the aorta had the present system not been delivered (or compared to the mRNA or protein level of COL2A1 in a control sample where the present method has not been employed). In one embodiment, the therapeutic agent is a SMAD, including, but not limited to, SMAD1, SMAD2, SMAD3, SMAD4, SMAD5, SMAD6, SMAD7, and SMAD 8. In another embodiment, the therapeutic agent is FOXP3. The therapeutic agent may be any agent described herein.

The present methods and systems may decrease the mRNA or protein level of COL1A2 (collagen 1A2), COL2A1 (collagen 2A1) and/or connective tissue growth factor (CTGF) in the liver of a subject by from about 5% to about 90%, from about 10% to about 80%, from about 10° to about 40%, from about 15% to about 70%, from about 20% to about 60%, from about 10% to about 20%, from about 5° to about 15%, from about 20° to about 30%, from about 30° to about 40%, from about 40° to about 50%, from about 50° to about 60%, from about 60° to about 70%, from about 30° to about 50%, from about 50° to about 80%, about 10%, about 15%, about 20%, about 25%, about 30%, about 40%, about 45%, about 50%, about 60%, about 65%, about 70%, or about 75%, compared to the mRNA or protein level of COL1A2, COL2A1 or CTGF in the liver had the present system not been delivered (or compared to the mRNA or protein level of COL1A2, COL2A1 or CTGF in a control sample where the present method has not been employed). In one embodiment, the therapeutic agent is a SMAD, including, but not limited to, SMAD1, SMAD2, SMAD3, SMAD4, SMAD5, SMAD6, SMAD7, and SMAD 8. In another embodiment, the therapeutic agent is FOXP3.The therapeutic agent may be any agent described herein.

Non-limiting examples of therapeutic agents also include a cytokine, a chemokine, an immunomodulatory molecule, a prodrug converting enzyme, an angiogenesis inhibitor, an angiogenesis promoter, a toxin, an antitumor agent, a mitosis inhibitor protein, an antimitotic agent, a transporter protein, tissue factor, enzymes, blood derivatives, hormones, lymphokines, interleukins, interferons, tumor necrosis factors, growth factors, neurotransmitters or their precursors or synthetic enzymes, trophic factors (such as BDNF, CNTF, NGF, IGF, GMF, alpha-FGF, beta-FGF, NT3, NTS, and HARP/pleiotrophin), apolipoproteins (such as ApoAI, ApoAIV, ApoE, dystrophin or a minidystrophin), the CFTR protein associated with cystic fibrosis, intrabodies, tumor-suppressing genes such as p53, Rb, RaplA, DCC, k-rev, coagulation factors such as factors VII, VIII, IX, DNA repair factors, suicide agents which cause cell death, cytosine deaminase, and pro-apoptic agents.

The therapeutic gene encoding the therapeutic agent may be from human or other species.

The therapeutic gene may be wild-type or a mutant. Mutants can be created by introducing one or more nucleotide substitutions, additions or deletions. For example, mutations can be introduced by standard techniques, such as site-directed mutagenesis and PCR-mediated mutagenesis. When the therapeutic agent is a protein, conservative amino acid substitutions may be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid, asparagine, glutamine), uncharged polar side chains (e.g., glycine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

For treating atherosclerosis, anti-inflammatory cytokines such as transforming growth factor beta 1 (TGFβ1) or interleukin 10 (IL-10) might be used for therapeutic effect. The use of disease-specific transcriptional promoters is one approach to give reduced and selective expression. Such disease-specific gene expression can direct expression to the site of disease where it is most needed and, at the same time, limit overall production. The disease-specific expression of IL-10 can be contemplated to lower the possibility of significant adverse reactions (22, 27).

Variants, analogs, or fuison proteins of a therapeutic agent may be used. In one embodiment, the therapeutic agent comprises or consists of the nucleotide sequence of SEQ ID NO: 2. In another embodiment, the therapeutic agent comprises or consists of a nucleotide sequence about 60% to about 100%, about 70% to about 100%, about 80% to about 100%, about 85% to about 100%, about 90% to about 100%, about 95% to about 100%, about 98% to about 100%, about 60%, about 70%, about 80%, about 85%, about 90%, about 95%, about 98%, or about 100% identical, to the nucleotide sequence of SEQ ID NO:2.

The therapeutic agent may be a small interfering RNA (siRNAs) or small-hairpin RNA (shRNA). The siRNA or shRNA may reduce or inhibit expression of a therapeutic target. SiRNAs may have 16-30 nucleotides, e.g., 16, 17, 18, 19, 20, 21, 22, 23, 24, 25, 26, 27, 28, 29, or 30 nucleotides. The siRNAs may have fewer than 16 or more than 30 nucleotides.

In another embodiment, the therapeutic agent is an antisense polynucleotide. The antisense polynucleotide may bind to a therapeutic target. An antisense oligonucleotide can be, for example, about 7, 10, 15, 20, 25, 30, 35, 40, 45, 50, 55, 60, 65, 70, 75, 80, or more nucleotides in length.

In a further embodiment, the therapeutic agent is a ribozyme. U.S. Pat. Nos. 8,592,368 and 5,093,246. Haselhoff et al., Nature 334: 585-591 (1988).

The therapeutic agent may be an antibody or antigen-binding portion thereof that is specific to a therapeutic target. The antibody or antigen-binding portion thereof may be the following: (a) a whole immunoglobulin molecule; (b) an scFv; (c) a Fab fragment; and (d) an F(ab′)2 etc. The antibody or antigen-binding portion thereof may be monoclonal, polyclonal, chimeric and humanized.

### Vectors

As used herein, the term “vector” refers to a polynucleotide capable of transporting another nucleic acid to which it has been linked. The present vectors can be, for example, a plasmid vector, a single- or double-stranded phage vector, or a single- or double-stranded RNA or DNA viral vector. Such vectors include, but are not limited to, chromosomal, episomal and virus-derived vectors, e.g., vectors derived from bacterial plasmids, bacteriophages, yeast episomes, yeast chromosomal elements, and viruses such as baculoviruses, papova viruses, SV40, vaccinia viruses, adenoviruses, fowl pox viruses, pseudorabies viruses and retroviruses, and vectors derived from combinations thereof, such as those derived from plasmid and bacteriophage genetic elements, cosmids and phagemids.

Expression vectors can be used to replicate and/or express the nucleotide sequence encoding a therapeutic agent in a target mammalian cell. A variety of expression vectors useful for introducing into cells the polynucleotides of the inventions are well known in the art. Viral vectors include, but are not limited to, adeno-associated virus, adenovirus, vaccinia virus, alphavirus, retrovirus and herpesvirus vectors.

In one embodiment, the vector is an adeno-viral associated virus (AAV) vector. Without wishing to be bound by any specific theory or mechanism, it is believed that the promoter can function in a disease-specific manner within the AAV vector due to the lack of strong promoter elements within the AAV inverted terminal repeats (35, 36). See also, e.g., Walsh et al., 1993, Proc. Soc. Exp. Biol. Med. 204:289-300; U.S. Pat. No. 5,436,146). AAV has been known to be an effective vector since 1984 (31, 32, 33-38), including delivering the IL10 gene in mouse atherogenesis models (26, 27).

Any of the AAV serotypes may be used, including, but not limited to, AAV1, AAV2, AAV3, AAV4, AAV5, AAV6, AAV7, AAV8, AAV9, AAV10, AAV11, etc.

The present vector may comprise wild-type or mutant AAV capsid (e.g., AAV2, AAV8, etc.) encoded by the AAV cap open reading frame (ORF).

In one embodiment, the mutant AAV capsid may have at least one tyrosine residue mutated (e.g., deleted, substituted with other amino acid residue(s), or having amino acid residue(s) inserted adjacent to the tyrosine). In another embodiment, there is an insertion of at least one amino acid residues into the cap ORF or the capsid gene. The mutant AAV capsid may have a combination of two or more of the above mutations.

For example, the AAV2 capsid may have Tyrosine-444 replaced by phenylalanine, and/or Tyrosine-730 replaced by phenylalanine. The AAV8 capsid may have Tyrosine-447 replaced by phenylalanine, and/or Tyrosine-733 replaced by phenylalanine. SEQ ID NO: 4 shows the amino acid sequence of AAV8 capsid with both tyrosine residues at positions 447 and 733 being substituted with phenylalanine residues. SEQ ID NO: 3 shows the nucleotide sequence of AAV8 capsid with both tyrosine residues at positions 447 and 733 being substituted with phenylalanine residues.

In another example, a 7-amino acid peptide having the sequence EYHHYNK (SEQ ID NO: 13) (refered to as “EYH” peptide herein), is inserted into the AAV capsid. The AAV2 capsid may have the peptide having the sequence EYHHYNK inserted after amino acid position 588. The AAV8 capsid may have the peptide having the sequence EYHHYNK inserted after amino acid position 590 (e.g., see SEQ ID NO: 5 for the nucleotide sequence of this embodiment; and SEQ ID NO: 6 for its amino acid sequence).

In a third embodiment, the AAV capsid may have a combination of two or more of the above modifications. SEQ ID NO: 7 shows the modified AAV8 capsid nucleotide sequence (SEQ ID NO: 8 shows the modified AAV8 capsid amino acid sequence) having both (1) Tyr447 and Tyr 733 mutated to Phe residues, and (2) 7-amino acid peptide (EYHHYNK) being inserted after position 590, as well as adjacent changes.

The present vector may comprise one or more of the following sequences: SEQ ID NO: 9, SEQ ID NO: 10, SEQ ID NO: 11, and SEQ ID NO: 12.

Adenoviruses are described in, e.g., Rosenfeld et al., 1991, Science 252:431-434; Rosenfeld et al., 1992, Cell 68:143-155; Mastrangeli et al., 1993, J. Clin. Invest. 91:225-234; Kozarsky and Wilson, 1993, Curr. Opin. Genetics Develop. 3:499-503; Bout et al., 1994, Human Gene Therapy 5:3-10; PCT Publication No. WO 94/12649; and Wang et al., 1995, Gene Therapy 2:775-783). U.S. Pat. No. 7,244,617.

Greater detail about retroviral vectors is available in Boesen et al., 1993, Biotherapy 6:291-302. In one embodiment, the present expression vector is a lentivirus (including human immunodeficiency virus (HIV)), which is a sub-type of retrovirus.

Plasmids that may be used as the present expression vector include, but are not limited to, pGL3, pCDM8 (Seed, 1987, “An LFA-3 cDNA encodes a phospholipid-linked membrane protein homologous to its receptor CD2”, Nature. 840-842) and pMT2PC (Kaufman et al., 1987, “Translational efficiency of polycistronic mRNAs and their utilization to express heterologous genes in mammalian cells”, EMBO J. 6:187-193). Any suitable plasmid may be used in the present invention.

Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome.

### Gene Therapy

The present expression vectors, compositions and methods may be used in gene therapy. Gene therapy refers to therapy performed by the administration to a subject of an expressed or expressible nucleic acid. Accordingly, the present invention provides for a method for treating or preventing a condition including cardiovascular diseases comprising administering to a patient in need thereof an effective amount of the present expression vector.

Any composition described for administration by gene therapy can also be useful, apart from gene therapy approaches, for in vitro or ex vivo manipulations.

Any of the methods for gene therapy available in the art can be used in accordance with the present invention (See, e.g., Goldspiel et al., 1993, Clinical Pharmacy 12:488-505; Grossman and Wilson, 1993, Curr. Opin. in Genetics and Devel. 3:110-114; Salmons and Gunzberg, 1993, Human Gene Therapy 4:129-141; Morgan and Anderson, 1993, Ann. Rev. Biochem. 62:191-217; Mulligan, 1993, Science 260:926-932; Tolstoshev, 1993, Ann. Rev. Pharmacol. Toxicol. 32:573-596; and Clowes et al., 1994, J. Clin. Invest. 93:644-651; Kiem et al., 1994, Blood 83:1467-1473, each of which is incorporated herein by reference). Gene therapy vectors can be administered to a subject systemically or locally by, for example, intravenous injection (See, e.g., U.S. Pat. No. 5,328,470) or by stereotactic injection (See, e.g., Chen et al., 1994, Proc Natl Acad Sci. 91:3054-57).

A pharmaceutical preparation of the gene therapy vector can comprise a gene therapy vector in an acceptable diluent, or can comprise a slow release matrix in which the gene delivery vehicle is embedded. Alternatively, where the complete gene delivery vector can be produced intact from recombinant cells, the pharmaceutical preparation can include one or more cells which produce the gene delivery system.

Gene therapy involves introducing a gene construct to cells in tissue culture or in vivo. Methods for introduction of polynucleotides of the invention to cells in vitro include, but are not limited to, electroporation, lipofection, calcium phosphate-mediated transfection, and viral infection. Usually, the method of transfer includes the transfer of a selectable marker to the cells. The cells are then placed under selection to isolate those cells that have taken up and are expressing the transferred gene. Those cells are then delivered to a subject. In another embodiment, the polynucleotides of the invention can be introduced into the target tissue as an implant, for example, in a polymer formulation (See, e.g., U.S. Pat. No. 5,702,717). In another embodiment, the polynucleotides of the invention can be targeted to the desired cells or tissues.

An expression vector can be delivered directly into a subject. In one embodiment, the polynucleotides of the invention can be injected directly into the target tissue or cell derivation site. Alternatively, a subject's cells are first transfected with an expression construct in vitro, after which the transfected cells are administered back into the subject (i.e., ex vivo gene therapy). Accordingly, the polynucleotides of the invention can be delivered in vivo or ex vivo to target cells. Several methods have been developed for delivering the polynucleotides of the invention to target cells or target tissues.

In a particular embodiment, a vector is introduced in vivo such that it is taken up by a cell and directs the expression of the therapeutic agent of the invention. Such a vector can remain episomal or can chromosomally integrate.

In a specific embodiment, an expression vector is administered directly in vivo, where the vector is expressed to produce the encoded product. This can be accomplished by any of numerous methods known in the art, e.g., by placing a nucleic acid of the invention in an appropriate expression vector such that, upon administration, the vector becomes intracellular and expresses a therapeutic agent. Such vectors can be internalized by using, for example, a defective or attenuated retroviral vector or other viral vectors that can infect mammalian cells (See, e.g., U.S. Pat. No. 4,980,286).

Alternatively, an expression construct comprising a nucleic acid of the invention can be injected directly into a target tissue as naked DNA. In another embodiment, an expression vector can be introduced intracellularly using microparticle bombardment, for example, by using a Biolistic gene gun (Dupont). In another embodiment, an expression construct comprising a nucleic acid of the invention can be coated with lipids, or cell-surface receptors, or transfecting agents, such that encapsulation in liposomes, microparticles, or microcapsules facilitates access to target tissues and/or entry into target cells. In yet another embodiment, an expression construct comprising a nucleic acid of the invention is linked to a polypeptide that is internalized in a subset of cells or is targeted to a particular cellular compartment. In a further embodiment, the linked polypeptide is a nuclear targeting sequence which targets the vector to the cell nucleus. In another embodiment, the linked polypeptide is a ligand that is internalized by receptor-mediated endocytosis in cells expressing the respective receptor for the ligand (See, e.g., Wu and Wu, 1987, J. Biol. Chem. 262:4429-4432).

In another embodiment, nucleic acid-ligand complexes can be formed such that the ligand comprises a fusogenic viral peptide which disrupts endosomes, thereby allowing the nucleic acid to avoid lysosomal degradation. In another embodiment, a nucleic acid of the invention can be targeted in vivo via a cell-specific receptor resulting in cell-specific uptake and expression (See, e.g., International Patent Publications WO 92/06180, WO 92/22635, WO 92/20316, WO 93/14188, and WO 93/2022. In yet another embodiment, a nucleic acid of the invention is introduced intracellularly and, by homologous recombination, can transiently or stably be incorporated within the host cell DNA, which then allows for its expression, (Koller and Smithies, 1989, Proc Natl Acad Sci. 86:8932-8935; Zijlstra et al., 1989, Nature 342:435-438).

In this embodiment, the expression vector is introduced into a cell prior to administration in vivo of the resulting recombinant cell. Such introduction can be carried out by any method known in the art, including, but not limited to, transfection, electroporation, microinjection, infection with a viral or bacteriophage vector comprising the polynucleotides, cell fusion, chromosome-mediated gene transfer, microcell-mediated gene transfer, and spheroplast fusion. Numerous techniques are known in the art for the introduction of foreign genes into cells (See, e.g., Maniatis et al., 1989; Current Protocols in Molecular Biology, John Wiley & Sons, 2000; Loeffler and Behr, 1993, Meth. Enzymol. 217:599-618; Cotten et al., 1993, Meth. Enzymol. 217:618-644; Cline, 1985, Pharmacol. Ther. 29:69-92) and can be used in accordance with the present invention.

The resulting recombinant cells can be delivered to a subject by various methods known in the art, and the skilled artisan would appreciate appropriate modes of administration. For example, intravenous administration may be the preferred mode of administration for recombinant hematopoietic stem cells. Similarly, the number of recombinant cells to be administered to a subject can be determined by one skilled in the art, and would include a consideration of factors such as the desired effect, the disease state, and the mode of administration.

### Conditions Treated

The present expression vectors, compositions and methods may be used to treat or prevent a cardiovascular disease, such as atherosclerosis, stenosis, restenosis, hypertension, heart failure, left ventricular hypertrophy (LVH), myocardial infarction, acute coronary syndrome, stroke, transient ischemic attack, impaired circulation, heart disease, cholesterol and plaque formation, ischemia, ischemia reperfusion injury, peripheral vascular disease, myocardial infection, cardiac disease (e.g., risk stratification of chest pain and interventional procedures), cardiopulmonary resuscitation, kidney failure, thrombosis (e.g., venous thrombosis, deep vein thrombosis, portal vein thrombosis, renal vein thrombosis, jugular vein thrombosis, cerebral venous sinus thrombosis, arterial thrombosis, etc.), thrombus formation, thrombotic event or complication, Budd-Chiari syndrome, Paget-Schroetter disease, coronary heart disease, coronary artery disease, need for coronary revascularization, peripheral artery disease, a pulmonary circulatory disease, pulmonary embolism, a cerebrovascular disease, cellular proliferation and endothelial dysfunction, graft occlusion or failure, need for or an adverse clinical outcome after peripheral bypass graft surgery, need for or an adverse clinical outcome after coronary artery bypass (CABG) surgery, failure or adverse outcome after angioplasty, internal mammary artery graft failure, vein graft failure, autologous vein grafts, vein graft occlusion, ischaemic diseases, intravascular coagulation, cerebrovascular disease, or any other cardiovascular disease related to obesity or an overweight condition.

Conditions that may also be treated using the present compositions and methods include diseases which are more prominent due to aging or increased inflammation, such as stroke, arthritis, and dementia. Schwarz et al., Identification of differentially expressed genes induced by transient ischemic stroke, Brain Res Mol Brain Res. 2002; 101(1-2):12-22. Simopoulou et al., Lectin-like oxidized low density lipoprotein receptor 1 (LOX-1) expression in human articular chondrocytes. Clin Exp Rheumatol. 2007, 25(4):605-12. Kakinuma et al., Lectin-like oxidized low-density lipoprotein receptor 1 mediates matrix metalloproteinase 3 synthesis enhanced by oxidized low-density lipoprotein in rheumatoid arthritis cartilage, Arthritis Rheum. 2004, 50(11):3495-503. Li et al., The new role of LOX-1 in hypertension induced neuronal apoptosis, Biochem Biophys Res Commun. 2012 September 7; 425(4):735-40.

Non-limiting examples of the conditions the present expression vectors, compositions and methods may be used to treat or prevent include rheumatoid arthritis, adenosine deaminase deficiency, hemophilia, cystic fibrosis, and hyperproliferative disorders (e.g., cancer etc.), bone resorption, osteoporosis, osteoarthritis, periodontitis, hypochlorhydia and neuromyelitis optica, insulin resistance, glucose intolerance, diabetes mellitus, hyperglycemia, hyperlipidemia, dyslipidemia, hypercholesteremia, hypertriglyceridemia, hyperinsulinemia, diabetic dyslipidemia, HIV-related lipodystrophy, peripheral vessel disease, cholesterol gallstones, menstrual abnormalities, infertility, polycystic ovaries, osteoarthritis, sleep apnea, metabolic syndrome (Syndrome X), type II diabetes, diabetic complications including diabetic neuropathy, nephropathy, retinopathy, cataracts, any other asthmatic and pulmonary diseases related to obesity and any other viral infection similar to HIV-1 infection related diseases.

### Pharmaceutical Compositions

The present invention provides for a pharmaceutical composition comprising a therapeutically effective amount of the present expression vector.

The present agents or pharmaceutical compositions may be administered by any route, including, without limitation, oral, transdermal, ocular, intraperitoneal, intravenous, ICV, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The present composition may be administered parenterally or systemically.

The pharmaceutical compositions of the present invention can be, e.g., in a solid, semi-solid, or liquid formulation. Intranasal formulation can be delivered as a spray or in a drop; inhalation formulation can be delivered using a nebulizer or similar device; topical formulation may be in the form of gel, ointment, paste, lotion, cream, poultice, cataplasm, plaster, dermal patch aerosol, etc.; transdermal formulation may be administered via a transdermal patch or iontorphoresis. Compositions can also take the form of tablets, pills, capsules, semisolids, powders, sustained release formulations, solutions, emulsions, suspensions, elixirs, aerosols, chewing bars or any other appropriate compositions.

The composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

To prepare such pharmaceutical compositions, one or more of compound of the present invention may be mixed with a pharmaceutical acceptable excipient, e.g., a carrier, adjuvant and/or diluent, according to conventional pharmaceutical compounding techniques.

Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers, preservatives and adjuvants, see Remington's Pharmaceutical Sciences, edited by E. W. Martin (Mack Publishing Company, 18th ed., 1990). Additional excipients, for example sweetening, flavoring and coloring agents, may also be present.

The pharmaceutically acceptable excipient may be selected from the group consisting of fillers, e.g. sugars and/or sugar alcohols, e.g. lactose, sorbitol, mannitol, maltodextrin, etc.; surfactants, e.g. sodium lauryle sulfate, Brij 96 or Tween 80; disintegrants, e.g. sodium starch glycolate, maize starch or derivatives thereof; binder, e.g. povidone, crosspovidone, polyvinylalcohols, hydroxypropylmethylcellulose; lubricants, e.g. stearic acid or its salts; flowability enhancers, e.g. silicium dioxide; sweeteners, e.g. aspartame; and/or colorants. Pharmaceutically acceptable carriers include any and all clinically useful solvents, dispersion media, coatings, antibacterial and antifungal agents, isotonic and absorption delaying agents and the like.

The pharmaceutical composition may contain excipients for modifying, maintaining or preserving, for example, the pH, osmolarity, viscosity, clarity, color, isotonicity, odor, sterility, stability, rate of dissolution or release, adsorption or penetration of the composition. Suitable excipients include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HC1, citrates, phosphates, other organic acids); bulking agents (such as mannitol or glycine), chelating agents (such as ethylenediamine tetraacetic acid (EDTA), ethylene glycol tetraacetic acid (EGTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

Oral dosage forms may be tablets, capsules, bars, sachets, granules, syrups and aqueous or oily suspensions. Tablets may be formed form a mixture of the active compounds with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound, may be prepared by known methods. The contents of the capsule may be formulated using known methods so as to give sustained release of the active compounds. Other dosage forms for oral administration include, for example, aqueous suspensions containing the active compounds in an aqueous medium in the presence of a non-toxic suspending agent such as sodium carboxymethylcellulose, and oily suspensions containing the active compounds in a suitable vegetable oil, for example arachis oil. The active compounds may be formulated into granules with or without additional excipients. The granules may be ingested directly by the patient or they may be added to a suitable liquid carrier (e.g. water) before ingestion. The granules may contain disintegrants, e.g. an effervescent pair formed from an acid and a carbonate or bicarbonate salt to facilitate dispersion in the liquid medium. U.S. Pat. No. 8,263,662.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

Additional compositions include formulations in sustained or controlled delivery, such as using liposome or micelle carriers, bioerodible microparticles or porous beads and depot injections.

The present compound(s) or composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions of the invention can be carried out using any of several standard methods including, but not limited to, continuous infusion, bolus injection, intermittent infusion, inhalation, or combinations of these methods. For example, one mode of administration that can be used involves continuous intravenous infusion. The infusion of the compositions of the invention can, if desired, be preceded by a bolus injection.

### Kits

The present invention also provides for a kit for use in the treatment or prevention of a cardiovascular disease or other conditions. Kits according to the invention include package(s) (e.g., vessels) comprising agents or compositions of the invention. The kit may include an expression vector of the present invention. The expression vector may be present in unit dosage forms.

Examples of pharmaceutical packaging materials include, but are not limited to, bottles, tubes, inhalers, pumps, bags, vials, containers, syringes, bottles, and any packaging material suitable for a selected formulation and intended mode of administration and treatment.

Kits can contain instructions for administering agents or compositions of the invention to a patient. Kits also can comprise instructions for uses of the present agents or compositions. Kits also can contain labeling or product inserts for the present expression vectors or compositions. The kits also can include buffers for preparing solutions for conducting the methods. The instruction of the kits may state that the expression vector drives disease-specific expression of a therapeutic agent.

Subjects, which may be treated according to the present invention include all animals which may benefit from administration of the agents of the present invention. Such subjects include mammals, preferably humans, but can also be an animal such as dogs and cats, farm animals such as cows, pigs, sheep, horses, goats and the like, and laboratory animals (e.g., rats, mice, guinea pigs, and the like).

The following examples of specific aspects for carrying out the present invention are offered for illustrative purposes only, and are not intended to limit the scope of the present invention in any way.

### EXAMPLES

### Example 1

### Disease-Limited Interleukin 10 (IL10)-AAV Gene Therapy for Cardiovascular Disease

Many therapeutic genes, such as transforming growth factor beta 1 (TGFβ1) and interleukin 10 (IL 10), have some level of adverse effects. The use of a disease-specific promoter may minimize the “downside” of these genes, yet provide enough local expression at the site of disease to effect a cure. The lectin-like oxidized low density lipoprotein receptor 1 (LOX1, oxidized LDL receptor 1, OLR1) is transcriptionally up-regulated early on in the disease process by a number of cell types, predating and predicting the sites of future disease. In this study, an adeno-associated virus vector (AAV2 backbone), using the AAV8 capsid, and containing the full length LOX1 promoter (LOX1pr; 2.4 kb), was generated and assayed for its ability to express human interleukin 10 (hIL10) for anti-atherosclerotic effect in low density lipoprotein receptor knockout (LDLR KO) mice on high cholesterol diet (HCD). The cytomegalovirus immediate early (CMVpr) promoter was used for comparison in a similarly structured vector. Both AAV2/8.LOX1pr-hIL10 and AAV2/8.CMVpr-hIL10 were found to give statistically equal efficacy in their down-regulation of atherogenesis as measured by aortic systolic blood velocity, aortic cross sectional area, and aortic wall thickness. This is a direct comparison of a constitutive promoter (CMVpr) with a disease-specific promoter (LOX1pr) in a therapeutic context.

### Materials and Methods

### Generation of Recombinant AAV Virus.

We utilized the full length 2.4 kb LOX1 promoter as has been previously characterized (23-31) To generate the AAV/LOX1pr-IL10, the full length Lox1 promoter (nt -2402 to +9) was amplified from human 293 cell by PCR using the primers: upstream 5′-ATATGCATCTTTCTTATTTGGGGGAAG-3′ (SEQ ID NO: 14) and downstream 5′-ATACGCGTACTAAAAATATGTGAGCTTCTG-3′ (SEQ ID NO: 15). Nsi I and Mlu I (underlined) sites were included in the primers to allow easy ligation in front of the hIL10 gene within the gutted AAV2 plasmid dl3-97. Construction and generation of AAV/Neo and AAV/CMVpr-hIL10 recombinant virus have been described previously (28, 37, 50). For comparison we utilized the cytomegalovirus immediate early constitutive promoter (CMVpr), which is well studied (33, 34). The virus stocks were generated and titered by dot blot hybridization as described previously (18, 32, 50). The titers were calculated to be about 1·10⁹ encapsidated genomes per ml (e.g./ml).

### Animal Treatments.

The low density lipoprotein receptor (LDLR) knockout mouse (B6; 129S7-Ldlr^{tm1Her}/J) was used in these studies, and was purchased from Jackson Laboratories (Bar Harbor, Me., USA). Two groups of male mice weighing 16-20 grams were tail vein injected with AAV2/8.LOX1pr-hIL10, AAV2/8.CMVpr-hIL10, or AAV2/8.SV40pr-Neo virus, each at a titer of 1×10⁹ e.g./ml via tail vein injections of 200 ul virus/mouse, followed by two booster injections at an interval of approximately 5 days. High cholesterol diet (HCD) of 4% cholesterol and 10% Coco butter diet (Harlan Teklad, Madison, Wis., USA) was then provided on the day of first injection and continuously maintained for twenty weeks. This high fat level HCD diet was used to ensure the development of atherosclerosis. Another control group included mice fed a normal chow diet. All animals were weighed weekly starting at 16 weeks post-injection.

### High Resolution Ultrasound Imaging

The Vevo 770 High-Resolution Imaging system (Visualsonics, Toronto, Canada) was used for ultrasound imaging, using an RMV 707B transducer, having a center frequency of 30 MHz. Animals were prepared as described earlier (50). Briefly, eight to ten mice from each group were anesthetized with 1.5% isoflurane (Isothesia, Abbot Laboratories, Chicago, USA), with supplemental oxygen and laid supine on a thermostatically heated platform. Their legs were taped to ECG electrodes for cardiac function monitoring. A shaver was used to remove abdominal hair along with a chemical hair remover (Church & Dwight Co, Inc., NJ, USA) and pre-warmed US gel (Medline Industries, Inc., Mundelein, USA) was then spread over the skin as a coupling medium for the transducer. The thoracic/abdominal region of the aorta was visualized, below the aortic arches to the diaphragm. Image acquisition was started in the B-mode, where, a long axis view was used to visualize the length of the aorta. The scanhead probe was then turned 90° for a short-axis view to obtain pictures of the cross-sectional area of the aorta. Individual frames and cine loops (300 frames) were also acquired at all levels of the aorta both in long axis and short axis view and recorded at distances of 1 mm throughout the length of the aorta. The flow velocity, orientation of the abdominal aorta on ultrasound, was accomplished by tilting the platform and the head of mouse down with the transducer probe towards the feet and tail of the mouse. The described positioning ensured that the Doppler angle was less than 60° for accurate measurements of blood flow velocity in the pulse-wave Doppler (PW) mode within the aorta. Measurements and data analysis was performed off-line on the longitudinal and transverse images using the customized version of Vevo770 Analytical Software. It took approximately 25-30 minutes to carry out the complete imaging for each mouse.

### Measurement of Plasma Cholesterol

The Veterans Animal Laboratory (VAMU) determined the plasma levels of total cholesterol for the animal groups, measured by VetScan VS2 (ABAXIS, Union City, Calif.).

hIL10 Gene Expression Analysis Using Real-Time Quantitative Reverse Transcription PCR (QRT-PCR)

Six mice from each group were sacrificed and total aortic RNA was extracted with TRIzol extraction (Invitrogen Carlsbad, Calif.) according to the manufacturer's instructions. cDNA was generated using random hexamer primers and RNase H-reverse transcriptase (Invitrogen, Carlsbad, Calif.). QRT-PCR was then performed using the Applied Biosystems Fast 7900HT real-time PCR system (Applied Biosystems, Foster City, Calif.) as described (33). We designed qRT-PCR specific primers for analyzing human IL10 using Probe-Finder (http://www.roche-applied-science.com) web-based software from Human and Mouse Universal ProbeLibrary from Roche Applied Science. The results were further analyzed using SDS 2.3 relative quantification (RQ) manager software. The comparative threshold cycles (Ct) values were normalized for the βactin reference gene and then compared with a calibrator by the 2^{-ΔΔCt} method.

AAV2/8 Delivers hIL10

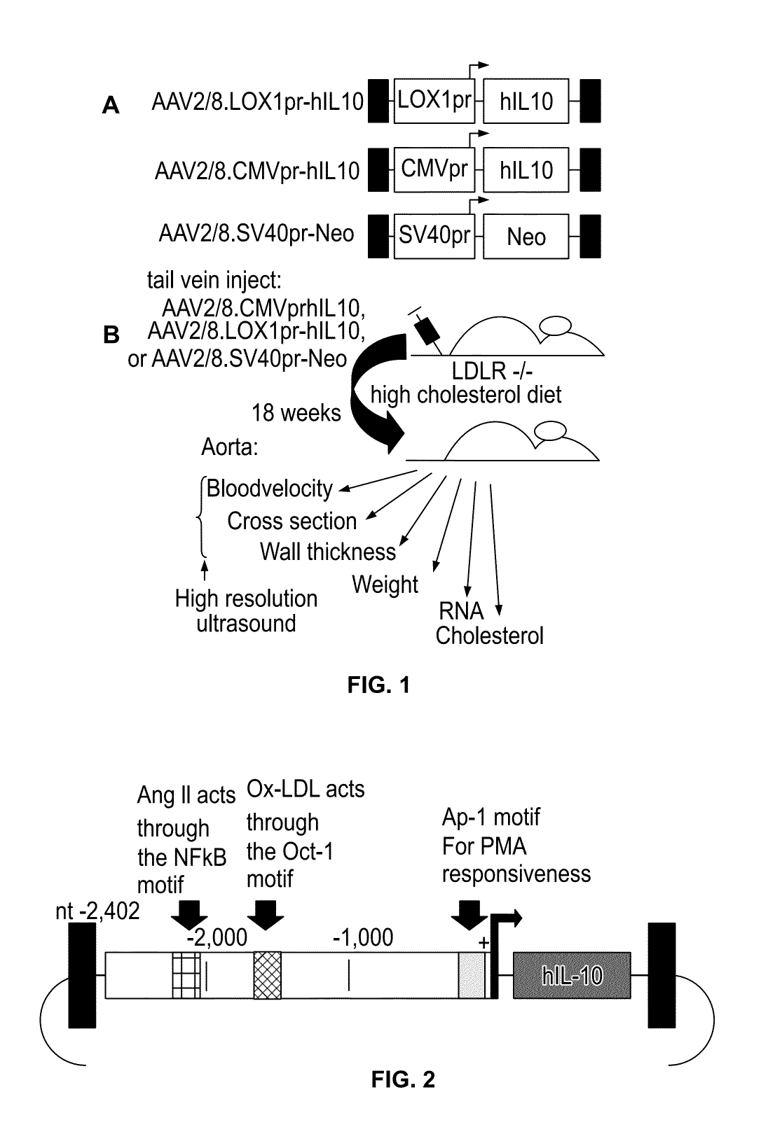

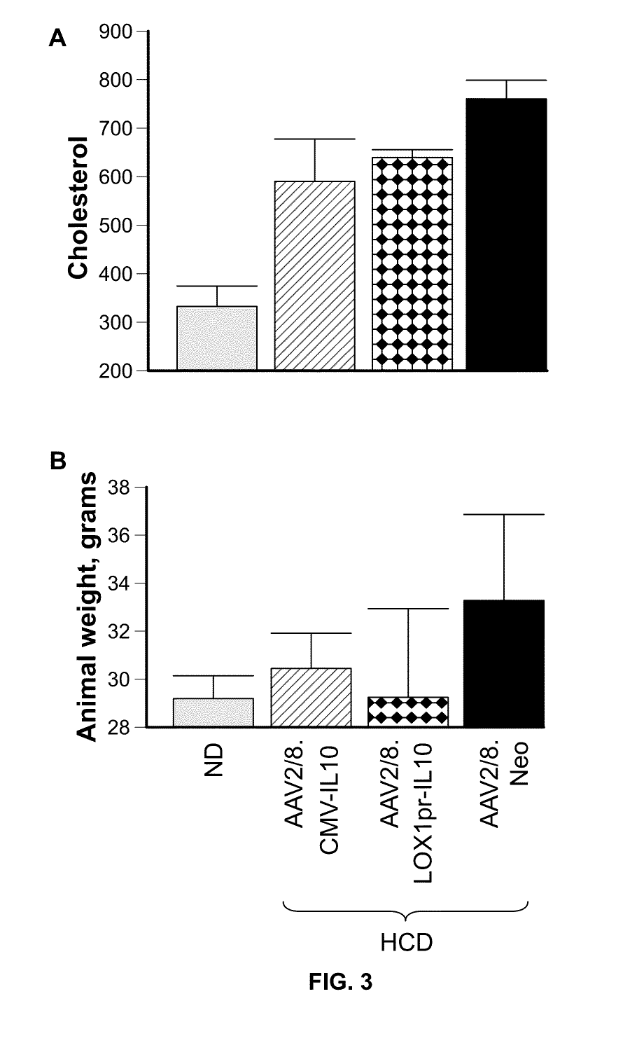

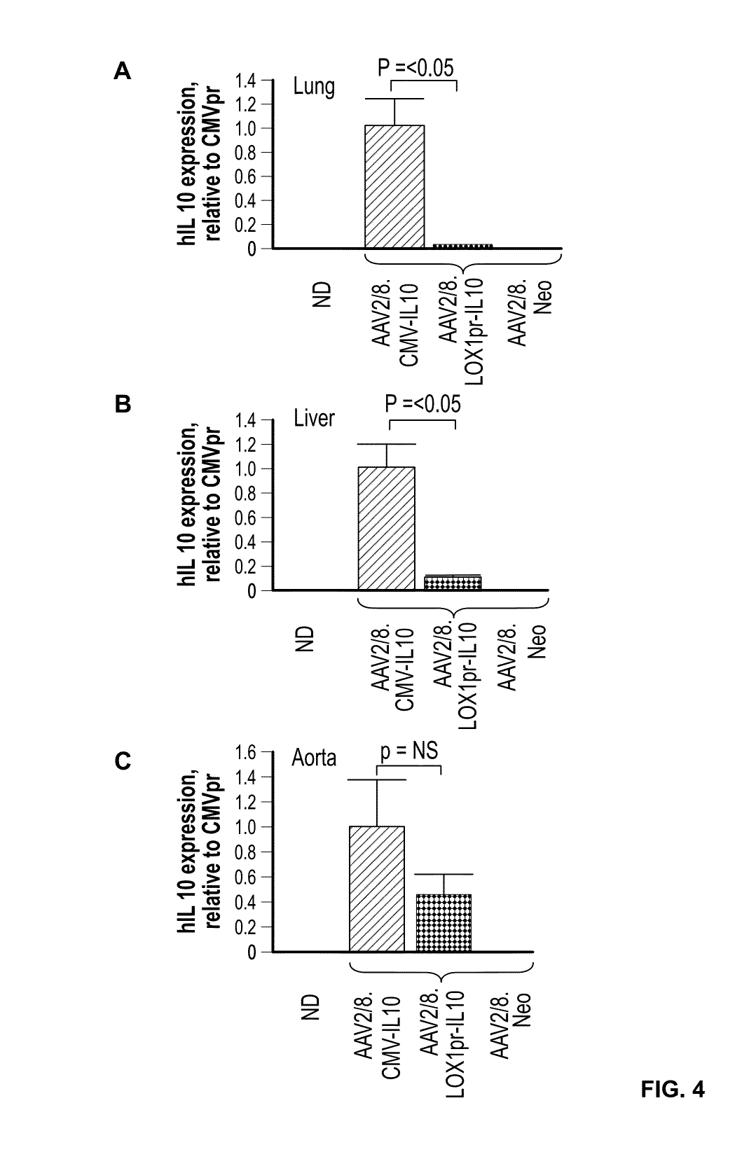

In order to investigate the use of a disease-specific promoter to limit the possibility of adverse reactions to systemic IL-10 expression (18-20), the efficacy of AAV2/8.LOX1pr-IL10 was compared to AAV2/8.CMVpr-IL10 delivery in LDLR KO mice and then placed on high cholesterol diet (HCD). An AAV/Neomycin resistance gene (Neo) vector (AAV2/8.SV40pr-Neo) was also used as a non-therapeutic, null control. Vector structures are shown in FIG. 1A and the overall experimental scheme in FIG. 1B. A more detailed view of the AAV.LOX1pr-hIL10 vector plasmid is shown in FIG. 2. At the time of harvest, a portion of mice were sacrificed to determine the success of gene delivery by analyzing hIL10 mRNA expression in the aorta using qRT-PCR analysis. This analysis utilized mRNA isolated from 6 mouse aortas from each group, harvested at week 20. Representative results for LOX1pr- and CMVpr-hIL10-treated mice are shown in FIG. 3, and both vectors were observed to be highly expressed in aortas at a level of appropriately 0.1-0.2% that of β-actin. Moreover, while the expression by CMVpr and LOX1pr were statistically similar, it is apparent that the LOX1pr expression trended lower, less than 50% than that of CMVpr.

Both Therapeutic CMVpr-hIL10 and LOX1pr-hIL10 Transgene Delivery Inhibits Aortic Blood Flow Velocity with Equal Efficacy.

Having demonstrated significant gene delivery/expression for both CMVpr-hIL10 orLOX1pr-hIL10, we then studied the effects of the transgene. FIG. 3A shows that CMVpr-hIL10 and LOX1pr-hIL10-transgene-HCD-treated animals had similar levels of total cholesterol levels, and were comparable to Neo-treated HCD-fed animals, however hIL-10 treated animals trended slightly lower as has been seen previously. Regarding animal weights, all treatments were statistically the same as normal diet (ND) controls but trended slightly lower in both hIL10-treated animal groups (FIG. 3B).

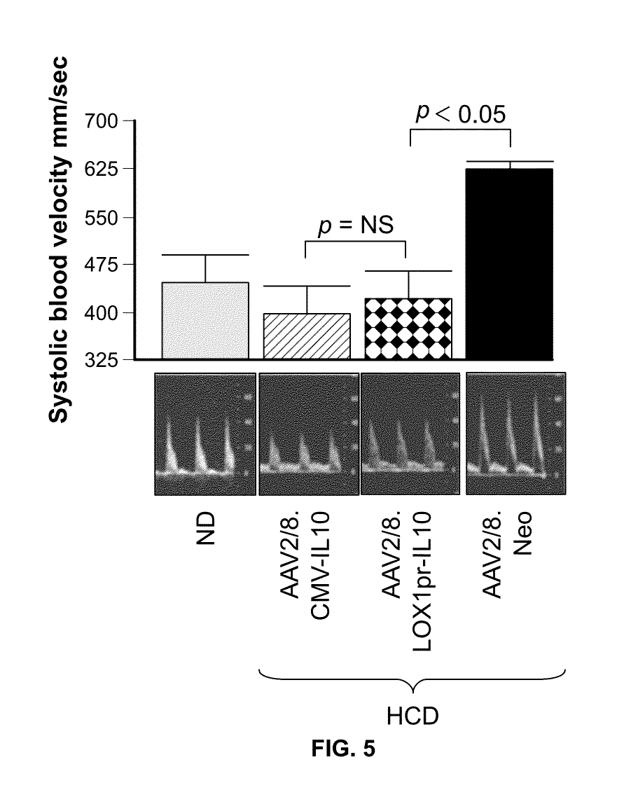

We analyzed arterial blood flow velocity as an easy technique to calculate the extent of atherosclerosis in the mouse groups. The systolic blood velocity in the thoracic/abdominal region of the aorta was quantified by high resolution ultrasound (HRUS) imaging system Vevo 770 with measurements taken on eight-to-ten animals. FIG. 5 shows the quantified systolic blood velocity from five separate measurements on each animal. It can be seen that Neo-HCD-treated animals displayed significantly higher blood flow velocities in their aortas, consistent with a constricted aortic lumen. Additionally, both CMVpr- and LOX1pr-driven gene therapies (hIL-10) on HCD had markedly lower flow velocity than the Neo-HCD-treated group, and very similar to that of the ND fed control animals. Thus, as suggested by lower blood velocity, both CMVpr- and LOX1pr-treatments displayed an anti-atherosclerotic effect and were statistically similar in their degree efficacy.

Both Therapeutic CMVpr-hIL10 and LOX1pr-hIL10 Gene Delivery Inhibits Aortic Structural Changes Associated with Atherosclerosis with Equal Efficacy.

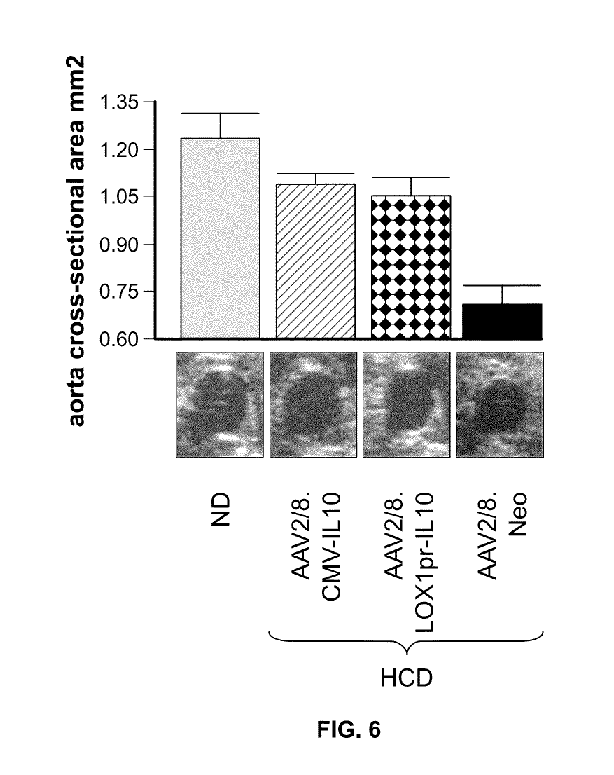

We then analyzed structural changes within the aortas of the various experimental groups by first measuring the aortic cross sectional area. Multiple measurements were taken from eight-to-ten animals in each group. The measurements were made in the same thoracic/abdominal site (see Materials and Methods). FIG. 6 shows the quantified results for the thoracic/abdominal region of the aorta. It can be seen that, the AAV/Neo-HCD-treated positive control animals had the smallest cross sectional lumen area, being consistent with higher atherosclerosis. In contrast, the two therapeutic treatments, both LOX1pr-hIL10-HCD and CMVpr-IL10, both on HCD, had much larger lumens than Neo-treated-HCD controls and this difference was statistically significant. The ND group trended to have the largest lumens.

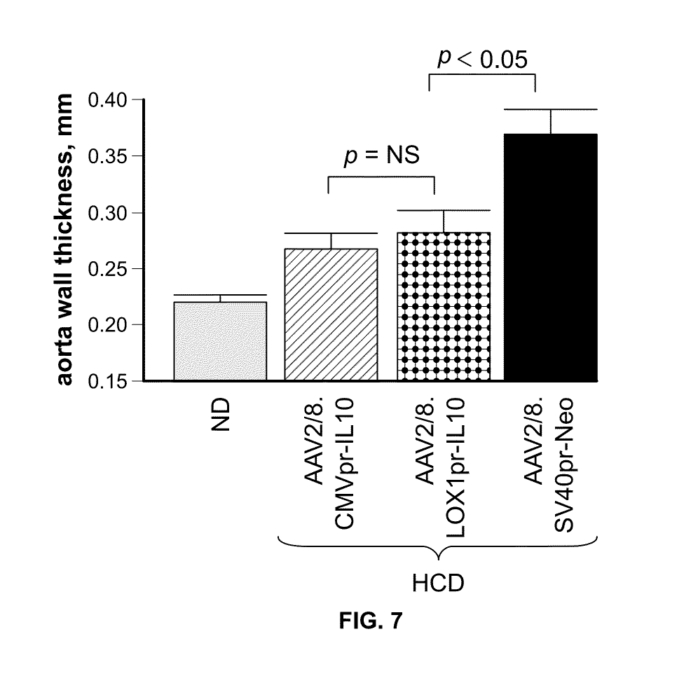

The wall thickness of the thoracic region of the aorta was also measured. FIG. 7 shows the quantified results for the thoracic region of the aorta. The Neo-HCD-treated animals displayed the thickest aortic walls, while both CMVpr- and LOX1pr-hIL10 vector-HCD-treatments were efficacious (p=<0.05), and were statistically similar to each other (p=NS. As expected, the ND group had the thinnest aortic walls. Thus all three measurements by HRUS (aortic systolic blood velocity, lumen size, and wall thickness) showed that the LOX1pr-driven vector was equally efficacious to the CMVpr version.

### Discussion

Here we demonstrate that an AAV2/8.LOX1pr-hIL10 vector was effective in inhibiting atherogenesis in LDLR KO mice on HCD, and was statistically equal to the inhibition derived from an AAV2/8.CMV-IL10 vector delivery. This is a direct comparison of a disease-specific promoter to a general expression promoter for determination of level of efficacy. The cytomegalovirus immediate early promoter is perhaps the most used promoter in gene delivery experiments. Thus this study establishes that disease-specific promoters are still able to express therapeutic transgenes to efficacious levels. It has been shown that the 2.4 kb LOX1 promoter fragment has significant activity and responsiveness to disease-associated stimuli as does the wt LOX-1 promoter. Normally the basal level LOX1 promoter activity is very low (44-48). In the future, we will also utilize a non-secreted marker gene to observe the activity of the vector bound disease-specific promoter. It is likely that OxLDL plays a role as we enhanced this agent with the HCD, but other stimulations are also possible, such as angiotensin II. The LOX1 promoter has been recently reviewed (35).

Moreover, in addition to LOX-1 as an atherogenesis-active promoter, there are a multiplicity of genes that are similarly up-regulated. DNA microarray experiments certainly drive this point home with many of genes being found regulated (52-56). The milieu of cells present with the artery wall during atherogenesis also changes over time (57-59). This issue should be taken into account when considering the exact disease-specific promoter to use, its strength, timing and possible cell preference. In normal arteries low monocyte populations exist within the arterial wall, while over time with the activation of the endothelium and recruitment of additional monocytes this number dramatically increases. Yet, our AAV vectors are systemically delivered before HCD-initiated disease. Thus transduction of the general monocyte population is likely taking place at some level, including some which will ultimately migrate into the arterial wall. Related to this issue, here, we have utilized a model of atherosclerosis in which atherogenesis is an active, ongoing process, induced by HCD, starting from a null standpoint. Another relevant model is the observation of regression of an established plaque.

We have studied the therapeutic use of AAV/IL10 and downstream STAT3 to limit atherogenesis in the LDLR KO mouse. This series of studies (37,59,60), along with those of others (36,37), has shown that IL10, and the IL10 signal transduction pathway, is beneficial for limiting atherogenesis by gene therapy. The first study, with IL10 being expressed from AAV2′s own p5 promoter, demonstrated that IL10 was effective in inhibiting plaque development in the LDLR KO mouse on HCD (18). In the second installment we demonstrated that STAT3, a gene through which IL10 signal transduction works, also confers anti-atherogenesis effect (59). STAT3 gene delivery could be useful as a substitute gene for IL10, hopefully with less adverse reactions than IL10. The third study examined IL10-plus-STAT3 dual gene delivery, studying the hypothesis that two genes in the same anti-inflammatory signal transduction pathway might act synergistically to limit atherosclerosis (60). Finally, going back to the use of original IL10 gene alone, here we demonstrate that AAV2/8.LOX1pr-IL10 delivery was equally efficacious as AAV2/8.CMVpr-IL10 delivery in limiting the development of atherogenesis. The LOX1pr structure in FIG. 2 shows that the DNA elements needed for Ox-LDL and AngII responsiveness represent only a small part of the full 2.4 kb promoter. This suggests some condensation of the LOX1 promoter might be undertaken to make this disease-specific promoter shorter and more useful for a wider variety of transgenes. This is important as the packaging of AAV genomes becomes more problematic as the size of the genome rises above 4.7 kb (61). However, additional disease-specific promoters and alterations to IL10 or other downstream genes besides STAT3, may be studied. This study also solidifies the usefulness and utility of adeno-associated virus vectors for cardiovascular gene therapy (37-40,59,60) as well as other diseases (34-37).

### Example 2

### Disease-Limited Interleukin 10 (IL10)-AAV Gene Therapy for Cardiovascular Disease—Modification of the AAV Vector

For the safe design of vectors carrying such powerful genes we have pioneered the “disease-specific promoter” gene expression approach in gene therapy. A disease-specific promoter will limit the expression and “adverse reactions” of IL 10, yet provides adequate expression at the site of disease to give treatment. The LOX1 gene is transcriptionally up-regulated early on in CVD (cardiovascular disease) in a number of cell types, including smooth muscle cells, predating and predicting the sites of future CVD.

We tested this disease-limited gene therapy hypothesis by studying an adeno-associated virus vector (AAV2 backbone), using the AAV8 capsid, and containing the full length LOX1 promoter (LOX1pr; 2.4 kb) driving expression of the human (h)IL10, for anti-atherosclerotic effect in low density lipoprotein receptor knockout (LDLR KO) mice on high cholesterol diet (HCD). We compared AAV2/8.LOX1pr-hIL10, with the LOX1pr driving hIL10 expression, to AAV2/8.CMVpr-hIL10. CMVpr is a strong constitutive promoter used for comparison (on all the time, positive control). The LOX1pr vector gave statistically equal efficacy to the CMVpr vector in down-regulating atherogenesis as measured by aortic cross sectional area, aortic wall thickness, and aortic systolic blood velocity, yet overall IL10 expression was significantly lower with the LOX1pr. In summary, the disease-specific LOX1pr gives therapeutic expression of IL10, but while maintaining overall lower IL10 expression.

With a disease-limited AAV2-LOX1pr-hIL10 DNA vector backbone capable of inhibiting atherosclerosis in a well respected animal model, our goal is to generate improved AAV-technologies for enhanced delivery of the AAV2-LOX1pr-IL10 vector backbone, and to test its safety in large animal models. In this study, we have three tasks. In Task 1, we will generate two specific improvements to the above proven therapeutic technology generating a more effective AAV2/8(cvd1). The improvements include EYH motif and tyrosine modifications in the AAV8 capsid, such as tyrosine substitution and EYH insertion into the AAV8 capsid gene. LOX1pr-IL10 vector, which will then be tested in explanted human arteries for improved gene delivery. In Task 2, this same vector will be analyzed for the titer needed for efficacy against HCD-induced atherosclerosis in a mouse model (e.g., C57BL/6 LDLR KO mice), and in Task 3, for the titer needed for efficacy in a rabbit model (e.g., familial hypercholesterolemic WHHL rabbits). The completion of these tasks will allow us to move on to toxicology analysis of our anti-atherosclerotic gene therapy agent before finally going to PHASE I clinical trials in patients.

### Specific Aims

We will modify the AAV8 capsid by two modifications and demonstrate its improved gene delivery capabilities and then assay the efficacy of disease-specific LOX1pr-hIL10 gene cassette by gene delivery into two animal models of atherosclerosis, the LDLR KO mouse and WHHL rabbit.

We will carry out the following tasks.
1) Generate single and dual modified (combined) tyrosine-minus, EYH-positive AAV8 capsid and test improvements in gene delivery in human placental umbilical cord blood vessels.
2) Determine titer of AAV2/8(cvd1).LOX1pr-IL10 vector needed for efficacy against HCD-induced atherosclerosis in LDLR KO mice.
3) Determine titer of AAV2/8(cvd1).LOX1pr-IL10 vector needed for efficacy against HCD-induced atherosclerosis in WHHL rabbits.
   Results may include the following:
1) Improve gene delivery into umbilical cord blood vessels smooth muscle cells at least about 3 fold above wild type AAV8 capsid vectors.
2) Inhibit HCD-induced atherosclerosis in LDLR KO mice by at least 70%.
3) Inhibit HCD-induced atherosclerosis in WHHL rabbits by at least 70%.

### Research Strategy

The targets for arterial gene delivery include the smooth muscle cells which express LOX-1.

### Disease-Limited IL10 Expression Using the LOX1 Promoter for Moderate Expression at the Site of Disease.

Using the LOX1 transcriptional promoter (LOX1pr) as a disease-specific promoter for expressing therapeutic genes to counter atherogenesis, we compared AAV2/8.LOX1pr-hIL10 gene delivery to AAV2/8.CMVpr-hIL10 gene delivery and find that both provide statistically similar efficacy.

The first technology will be to improve the AAV serotype 8 (AAV8) capsid protein with two modifications to increase gene delivery into arteries. These are amino acid tyrosine modifications, for capsid survival, and EYH peptide insertion, for very high frequency infection of smooth muscle cells.

### Approach

### Additional Advantages of AAV Vectors for CVD-Targeting Gene Therapy Treatments.

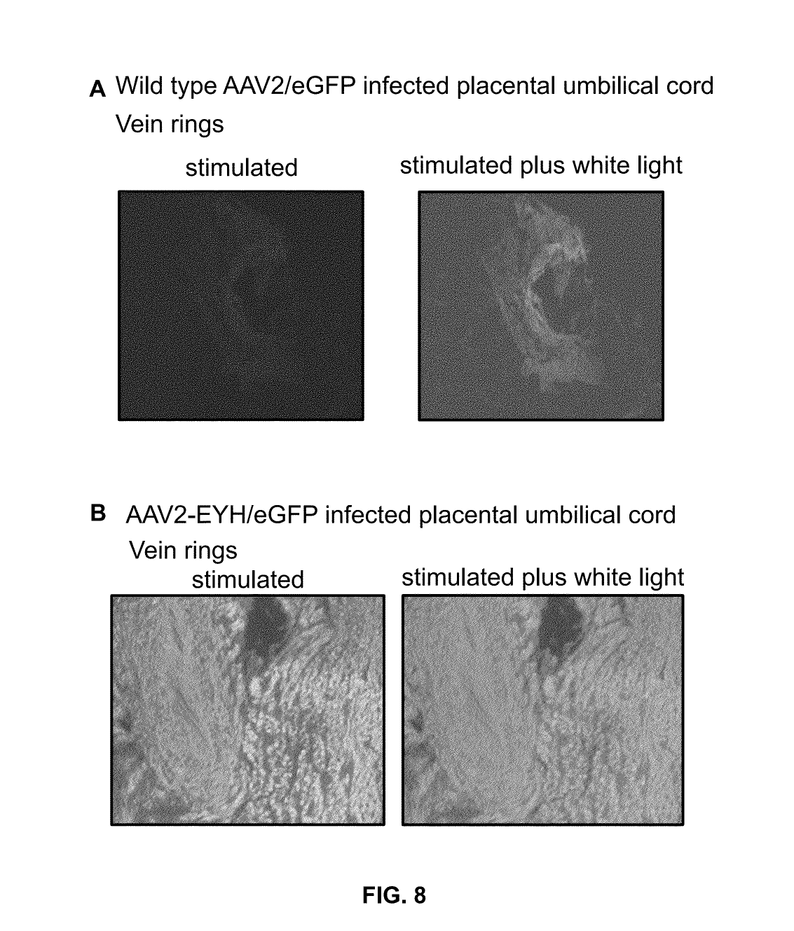

We and others (28-34) have shown that gene expression by AAV can be stably maintained for many months. Moreover, AAV is efficiently taken up by cardiovascular tissues (28-34,27,29). Richter et al. (50) showed efficient transduction in carotid arteries and that smooth muscle cells (SMCs) were the primary target. We found similar results (see FIG. 8), with uptake in the liver, lungs, kidneys, and blood vessels, via a single intravenous injection. In fact, AAV has the advantage of both high safety and long term expression.

Modification of AAV by insertion of EYH improves smooth muscle cell transduction. We modified the AAV2 capsid by inserting a peptide, EYHHYNK (EYH), which promotes infection of human vascular smooth muscle cells (VSMCs) in culture (66-69). We repeated the general AAV capsid modification by inserting the EYH peptide sequence, as a DNA sequence, into the AAV capsid gene at amino acid (AA) position 588. We then assayed for its ability to transduce human VSMCs within human placental umbilical cord vein rings in culture. As shown in FIG. 8, we observed that the EYH modification of the AAV2 capsid enhanced the transduction of the VSMCs.

This enhancement with the AAV2/EYH modification motivated us to employ this AAV capsid in the proposed research instead of AAV8, which we had used most recently. We also have additional capsid modifications that we may substitute for AAV2/EYH if they prove superior. For example, a series of tyrosine mutations within the AAV2 capsid gene which, when replaced by phenylalanine, was shown to allow for 10- to 50-fold higher transduction (69). The most important of these is at amino acid (aa) 730 (69). While AAV2 is the most used vector and, historically, the first AAV serotype to be used for gene delivery, we have used AAV8 extensively for arterial gene delivery (60-62). Our goals in this phase will be to translate both of these modifications into AAV8, and generate an AAV8/Y730F/EYH foundation vector. These modifications should give us a highly effective cardiovascular gene therapy vector from which we will deliver our therapeutic transgene, LOX1pr-IL10.

AAV2/8.LOX1pr-hIL10 Demonstrates that Disease-Limited Expression of IL10 Provides Efficacy Against Atherogenesis, yet with Significantly Lower Overall Expression than when Using the CMVpr.

This study solidifies the importance of the disease-specific gene therapy approach. This is also important as most gene therapy approaches use constitutive promoters which express genes in an “everywhere, all the time” approach. This is dangerous.

### Research Design and Methods

Task 1 is the generation of a new capsid, protein coat, a modification of AAV8, which gives higher transduction of smooth muscle cells in arteries. Our goal will be to improve gene delivery into umbilical cord blood vessels at least 5 fold above wild type AAV8 capsid vectors. This will give us a new, improved modified AAV8 vector for gene delivery into arteries. This improved CVD gene delivery vector will be referred to as AAV2/8(cvd1).LOX1pr-IL10. Task 2 is to determine titer of AAV2/8(cvd1).LOX1pr-IL10 vector, generated in Task 1, needed for efficacy against HCD-induced atherosclerosis in LDLR KO mice. Task 2′s findings will determine the level of virus needed to test in the Phase II toxicology studies, as a prelude before advancing to clinical studies. Task 3 is to determine titer of AAV2/8(cvd1).LOX1pr-IL10 vector, generated in Task 1, needed for efficacy against HCD-induced atherosclerosis in WHHL rabbits.

Task1: Modification of AAV8 by replacement of specific tyrosines and insertion of EYH peptide. Task 1 encompass two modifications of the AAV8 capsid. One modification will be the insertion of the EYH peptide, which improves infection to human vascular smooth muscle cells (see FIG. 8). Smooth muscle cells are in abundance in the normal aorta, as well in the advanced atheroma. Moreover, LOX-1 is up-regulated in arterial smooth muscle cells by a variety of insults such as oxidized low density lipoprotein and pro-inflammatory cytokines (73-74). The other will be the removal of certain tyrosine amino acids which are targeted by the cells protease systems for premature AAV capsid degradation. Thus our goals here will be transfer both of these valuable modifications into AAV8, and generate an AAV8/Y730F/EYH foundation vector. These modifications should give us a highly effective cardiovascular gene therapy vector from which we will be able to deliver our therapeutic transgene, LOX1pr-IL10. The actual DNA sequences of the AAV vector will NOT change. Thus the disease-limited expression of the AAV/LOX1pr-IL10 vector will function specifically as shown in Example 1. The only change will be in that the capsid modifications will result in a higher rate of delivery of these AAV vectors into the arteries.

We will modify the AAV8 capsid, as we have the AAV2 capsid, by inserting the EYHHYNK (EYH) peptide, which promotes binding to and infection of human vascular smooth muscle cells (VSMCs) in culture (68-70). We will utilize GenScript to generate the modifications within the AAV8 helper plasmid pDG8. While the original work was done with AAV2 infection of cultured human VSMCs, we repeated the general AAV capsid modification in AAV8 by inserting the EYH peptide sequence, as a DNA sequence, into the AAV capsid gene at amino acid (AA) position 590 (75). We then assayed for its ability to transduce human VSMCs within human placental umbilical cord vein rings in culture. As shown in FIG. 8, we observed that EYH enhanced the transduction of the VSMCs.

Modification of AAV8 by insertion of EYH and by replacing specific tyrosines with F/phenylalanine. We will then modify the AAV8 capsid to carry both of the modifications using GenScript and, again, test these, two modifications together for improved ability to transduce human VSMCs within human placental umbilical cord vein rings in culture.

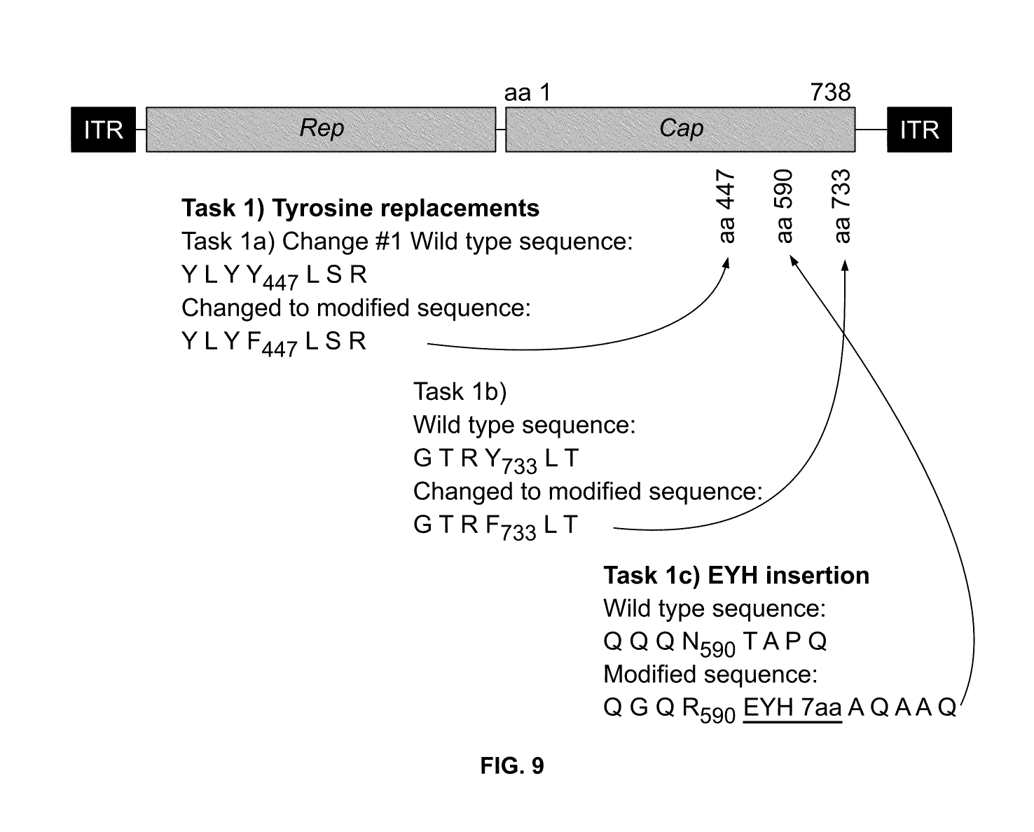

Modification of AAV8 by replacing specific tyrosines with F/phenylalanine. We will also have additional capsid modifications (Genscript) that we will carry out in addition to AAV8/EYH. A series of tyrosine mutations within the AAV2 capsid gene which, when replaced by phenylalanine, has been reported to allow for 10- to 50-fold higher transduction (69). The most important of these substitutions is at amino acid (aa) 444 and 730 (69). The analogous positions to aa444 and 730 of AAV2 in the AAV8 capsid protein are aa447 and 733 (see FIG. 9) (76). We will then generate AAV8/Y447F/Y733F/590-EYH virus which include the enhanced green florescence protein (eGFP) and assay for its ability to transduce human VSMCs within human placental umbilical cord vein rings in culture.

Hereafter AAV2/8(cvd1) refers to the modified AAV2/8-Y730E-EYH capsid derived from Task 1.

Task 2: Test efficacy in LDLR KO mice. The goal in Task 2 is to determine the amount, titer of, AAV2/8(cvd1).LOX1pr-IL10 (Kcardio-1) which is needed to give efficacy. We will use the LDLR KO mouse model to determine this efficacious titer. Three different doses, 10⁸, 10⁹, and 10¹⁰ e.g. (encapsidated genomes), of the AAV2/8(cvd1).LOX1pr-IL10. Our goal will be to inhibit atherosclerosis in the LDLR KO mouse model by 70% when placed on a high fat high cholesterol diet (HCD). The information on the virus amount needed for efficacy will then be utilized in the toxicology studies of Phase2. Methods are described in more detail in our previous publications (26-31,33,35,48,48,70-72).

Viral genome construction and AAV production. We have utilized standard protocols described by our laboratory to develop the AAV delivery vector AAV2/8(cvd1).LOX1pr-IL10 which is shown in FIG. 2.

Justification of animal numbers. Power analysis of from our earlier experiments indicates we will need animal group numbers of 12.

Animals, tissues, and transgene expression analysis. C57BL/6 LDLR KO mice (18-20 grams) in each of the seven treatment groups will include: AAV2/8(cvd1) refers to the modified AAV2/Y730F/EYH vector capsid derived from Task 1.
1) AAV2/8(cvd1)/Neo-normal diet (ND),
2) AAV2/8(cvd1)/Neo-HCD,
3) AAV2/8(cvd1)/empty (no gene)-HCD,
4) AAV2/8(cvd1).LOX1pr-IL10 at 10⁸ eg-HCD,
5) AAV2/8(cvd1).LOX1pr-IL10 at 10⁹ eg-HCD,
6) AAV2/8(cvd1).LOX1pr-IL10 at 10¹⁰ eg-HCD, and
7) ND ctrl mice (12 animals each).
   Neo- and IL10-gene treated animals will be fed a high-cholesterol diet (HCD) consisting of 4% cholesterol and 10% cocoa butter (Harlan Teklad, Madison, Wis.) on the day of the first injection. Mice fed normal chow diets will be included as controls. Animals will be weighed weekly.

Analysis of markers and cytokines in the aorta by quantitative real-time reverse transcriptase-PCR (qRT-PCR). This will be done by standard methodologies. The genes to be analyzed include macrophage markers such as CD68, CD36, ITGAM, and EMR. We expect these to be significantly down when AAV/IL10 is delivered. Other genes to be studied include CD4, CD8, CD 14, CD25, CD56, interleukin (IL)-2, interferon (IFN)-γ, IL-4, IL-7, IL-10, IL-17, transforming growth factor (TGF)-β1 andβ-actin (housekeeping control.

Analysis of markers and cytokines by immunohistochemistry. Standard methods will be utilized to visualize and measure the respective targets described (28,29,54,64,63). In particular, macrophage markers such as CD68, CD36, ITGAM, and EMR will be studied.

Analysis of atherogenesis by Oil Red O staining. This will be done by standard methodologies as we have done with Sudan IV.

Analysis of atherosclerosis by histologic cross-section. Harvested aortas will be fixed in formalin. Ten sections will be placed on slides and analyzed by imaging software (Image Pro Plus, Media Cybernetics) to quantify plaque formation.

High resolution ultrasound (HRUS) imaging. Ultrasound imaging will be performed with a Vevo 770 High-Resolution Imaging system (Visualsonics, Toronto, Canada) and a RMV 707B transducer with a center frequency of 30 MHz will be done by standard methodologies (27,54,63,64,72-91).

Measurement of plasma cholesterol. Plasma levels of total cholesterol in mice from respective treatment groups will be measured by VetScan VS2 (ABAXIS, Union City) at the Veterans Animal Laboratory (VAMU).

Analysis of atherogenesis by direct visualization. Whole dissected aortas will be fixed in 10% buffered formalin and inspected under a dissecting microscope, photographed, and photodigitally analyzed by Image J software (ImageJ, US National Institutes of Health, Bethesda, Md.).

Statistical analysis. Statistical analyses will be performed by nonparametric ANOVA analysis with the Statistical Program for Social Sciences version 11.5 for Windows (SPSS Inc, Chicago). If differences are detected between means, then the Newman-Keuls test will be used for multiple comparisons. Differences will considered significant for P<0.05.

Task 3: Test efficacy in WHHL rabbits. The goal in Task 3 is to determine the amount, titer of, AAV2/8(cvd1).LOX1pr-IL10 (Kcardio-1) which is needed to give efficacy. We will use a second animal model to determine efficacious titer, the Watanabe heritable hyperlipidemic (WHHL) rabbit (LDL receptor deficient). Three different doses, 10¹⁰, 10¹¹, and 10¹² eg (encapsidated genomes), of the AAV2/8(cvd1).LOX1pr-IL10 virus. Our goal will be to inhibit atherosclerosis in the WHHL rabbit model by 70% when placed on a high fat high cholesterol diet (HCD). WHHL rabbits will be infected at 2 months old before the development of atherosclerosis (HCD is not needed) and analyzed at 6 months when atherosclerosis is expected to cover about 35% of the aortic surface (. The information on the virus amount needed for efficacy will then utilized at a 10X/weight, ten fold level in the toxicology studies in Phase II. The animal groups will be: 1) AAV2/8(cvd1)/Neo-normal diet (ND), 2) AAV2/8(cvd1)/empty, 3) AAV2/8(cvd1).LOX1pr-IL10 at 10¹⁰ eg, 4) AAV2/8(cvd1).LOX1pr-IL10 at 10¹¹ eg, 5) AAV2/8(cvd1).LOX1pr-IL10 at 10¹² eg, and 6) ND ctrl rabbits (5 animals each). All experimental studies will be done similar to Task 2 with the LDLR KO mice, with the assays and readings being scaled up for the increased size of rabbits (1,500-2000 grams) over that of mice (18-24 grams).

### Example 3

### hSMAD3-AAV Gene Therapy for Cardiovascular Disease

Adeno-associated virus type 8 (AAV)/hSMAD3 was tail vein injected into low density lipoprotein receptor knockout (LDLR-KO) mice, and the mice were then placed on a high-cholesterol diet (HCD). The hSMAD3 delivery was associated with moderately lower plaque formation as measured by lower aortic systolic blood velocity, larger cross sectional area, and thinner wall thickness compared with NeoR gene-treated controls. hSMAD3 delivery also resulted in fewer aortic macrophages by immunohistochemistry for CD68 and ITGAM, and quantitative reverse transcriptase polymerase chain reaction analysis of EMR and ITGAM. Overall, aortic cytokine expression showed an enhancement of Th2 response (higher IL-4 and IL-10); while Th1 response (IL-12) was lower with hSMAD3 delivery. While TGFβ1 is often associated with increased fibrosis, AAV/hSMAD3 delivery exhibited no increase of collagen 1A2 and significantly lower 2A1 expression in the aorta compared with NeoR-delivery. Connective tissue growth factor (CTGF), a mediator of TGFβ1/SMAD3-induced fibrosis, was unchanged in hSMAD3-delivered aortas. In the liver, all three of these genes were down-regulated by hSMAD3 gene delivery.

These data suggest that AAV/hSMAD3 delivery provided a therapeutic effect without TGFβ1-induced fibrosis.

### Materials and Methods

### AAV Vector Construction and Virus Generation.

The human (h) SMAD3 and calcitonin gene-related protein (CGRP) cDNAs (obtained from Open Biosystems) were ligated downstream from the cytomegalovirus immediate early promoter within the gutted AAV vector dl3-97 to generate AAV/hSMAD3 and

AAV/hCGRP, respectively. The AAV/Neo vector has been described previously (26). AAV2/8 virus (AAV2 DNA in AAV8 virion) was produced using pDG8 helper (TransIT transfection of 6 μg each of pDG8 plus AAV vector plasmid into 10 cm plates of 293 cells), freeze-thawing the plates three times at 60 hours, the virus concentrated (pelleted) by ultracentrifugation, and titered by dot blot analysis by standard methodologies.

### Animal Treatments

LDLR KO mice (B6; 129S7-Ldlr^{tm1Her}/J) were purchased from Jackson Laboratories (Bar Harbor, Me., USA). Three groups of male mice, composed of ten animals each at 8 weeks old, were injected with AAV/Neo, AAV/hSMAD3 or AAV/hCGRP virus at a titer of 1×10¹⁰ eg/ml via tail vein with 200 μl virus per mouse, two booster injections were followed at an interval of 5-6 days. High cholesterol diet (HCD) of 4% cholesterol and 10% Coco butter diet (Harlan Teklad, Madison, Wis., USA) was provided from the first day of injection and maintained for the entire study period. Another group of mice fed with a normal diet was used as the control group. All experimental procedures conform to protocols approved by the Institutional Animal Care and Usage Committee of the Central Arkansas Veterans Health Care System at Little Rock.

### Ultrasound Imaging

The Vevo 770 High-Resolution Imaging system (Visualsonics, Toronto, Canada) with a RMV 707B transducer was used for all direct aortic examinations. Each mouse was anesthetized with inhalation of 1.5% isoflurane (Isothesia, Abbot Laboratories, Chicago, USA) with oxygen and placed supine on a thermostatically heated platform to maintain a constant body temperature. All legs were taped to ECG electrodes for cardiac function monitoring. Abdominal hair was removed using a chemical hair remover (Church & Dwight Co., Inc., NJ, USA), pre-warmed US gel (Medline Industries, Inc., Mundelein, USA) was spread over the skin as a coupling medium for the transducer. Image acquisition was started on B-mode; two levels of the vessel were visualized longitudinally: thoracic region and renal region. Thereafter, a short-axis view was taken to visualize the same arterial site in a crossectional view immediately. For each level, individual frames and cine loops (300 frames) were acquired and recorded at distances of 1 mm throughout the length of the aorta. Measurements and data analysis was performed off line using the customized version of Vevo 770 Analytical Software from both the longitudinal and transverse images.

### Tissue Sampling and Processing

At 20 weeks after first injection of virus and on HCD, mice were euthanized by CO2 exposure and exsanguinations to collect blood. Entire aorta for immunohistochemistry analysis were prepared. The aorta was flushed with saline solution and fixed in 10% neutral-buffered formalin (Sigma, St Louis, Mo., USA). After 24hrs, the fixed tissue was embedded by paraffin for sectioning. For real-time PCR analysis, the aorta sample were frozen in liquid nitrogen and stored in −80° C.

### Measurement of Plasma Cholesterol

Total plasma cholesterol of AAV/Neo and AAV/SMAD3 mice were measured by VetScan VS2 (Abaxis, Union City, Calif., USA) at the Veterans Animal Laboratory (VAMU).

RNA Isolation and Real-Time qRT-PCR.

Total aortic RNAs were extracted using Trizol reagent (Invitrogen Carlsbad, Calif.) and were treated with DNase I (Invitrogen, Carlsbad, Calif.). Then cDNA was synthesized using oligo(dT)18 primers and RNase H-reverse transcriptase (Invitrogen, Carlsbad, Calif.) according to the manufacturer's instructions. The specific primers for qPCR analysis were synthesized by Integrated DNA Technologies, Inc. (Coralville, Iowa). Real-Time Quantitative PCR was performed using SYBR Green PCR Master Mix kit on the Applied Biosystems Fast 7900HT real-time PCR system (Applied Biosystems, Foster City, Calif.). The results were analyzed with SDS 2.3 software.

### Results

### Establishing Gene and HCD Delivery.

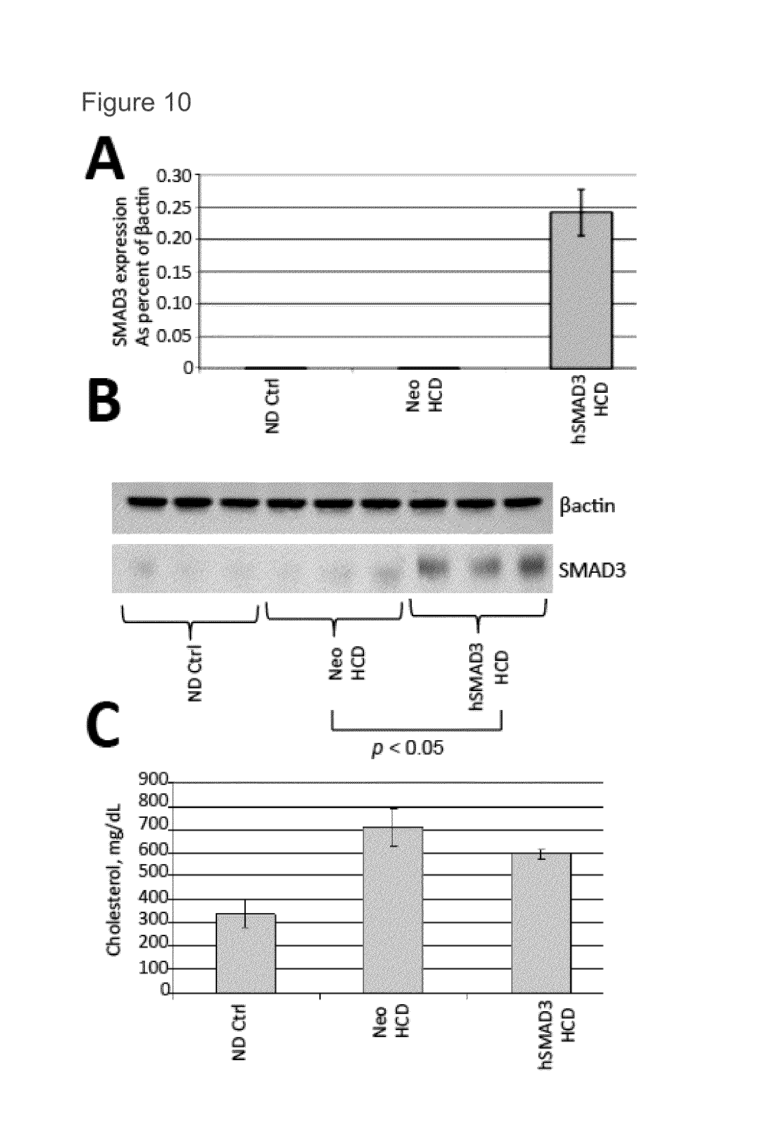

We evaluated whether hSMAD3 gene can serve as a substitute for TGFβ1, with lower frequency of systemic adverse effects than TGFβ1. AAV/hSMAD3 (AAV serotype 8) was delivered by tail vein injection and the animals placed on HCD. The animals were then harvested/high resolution ultrasound analyzed at 16-20 weeks post-injection/post HCD initiation. FIG. 10A shows that the delivery of hSMAD3 into the aorta was successful, being much higher in AAV/hSMAD3-treated animals. FIG. 10C shows that the blood cholesterol levels were high in both groups on a high cholesterol diet (HCD), but that the AAV/hSMAD3-treated animals were statistically lower, yet still similar in overall level.

### Analysis of Aortic Structure.

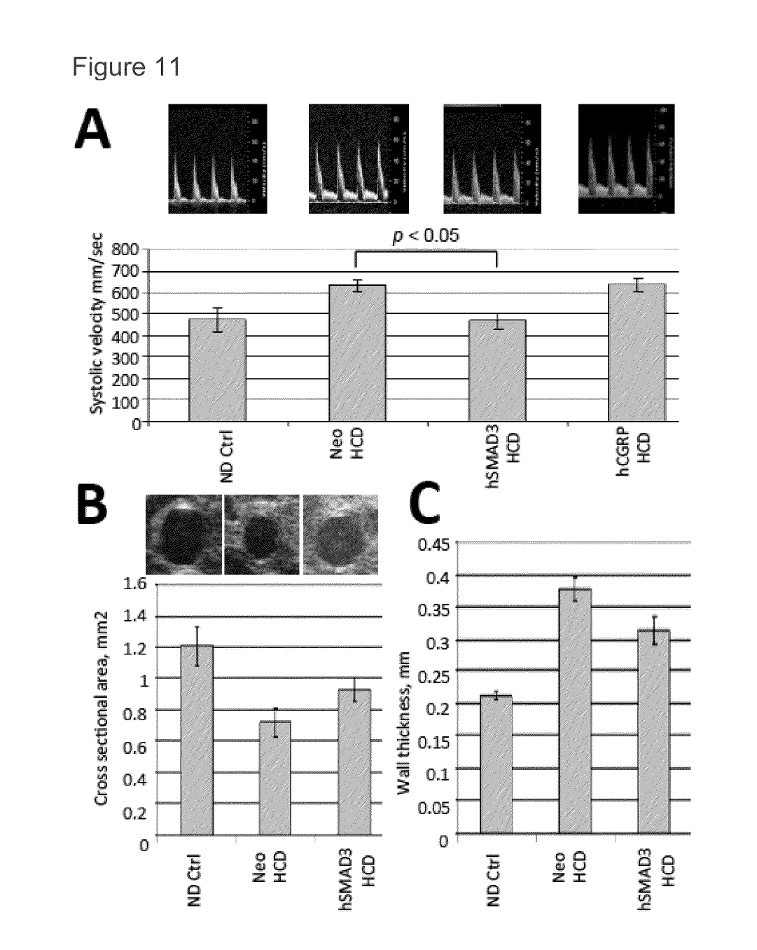

High resolution ultrasound (HRUS) was then used to analyze the aortas of at least three animals per group. FIG. 11C shows that the systolic thoracic region aortic blood velocity was significantly lower (p<0.05) in the hSMAD3/HCD-treated animals than the Neo/HCD-treated animals, consistent with less severe atherosclerosis. In sharp contrast to the effect of hSMAD3 delivery, another gene, calcitonin gene-related peptide (CGRP), had no effect on systolic aortic blood velocity. Thus, CGRP was dropped from further analysis. FIG. 11A shows that the aortic cross-sectional area was significantly larger in the hSMAD3/HCD-treated animals than the Neo/HCD-treated animals by HRUS. Moreover, HRUS, as shown in FIG. 11B, indicated that aortic wall thickness was significantly lower in the hSMAD3/HCD-treated animals than the Neo/HCD-treated animals, consistent with less severe atherosclerosis.

### Analysis of Macrophage Trafficking

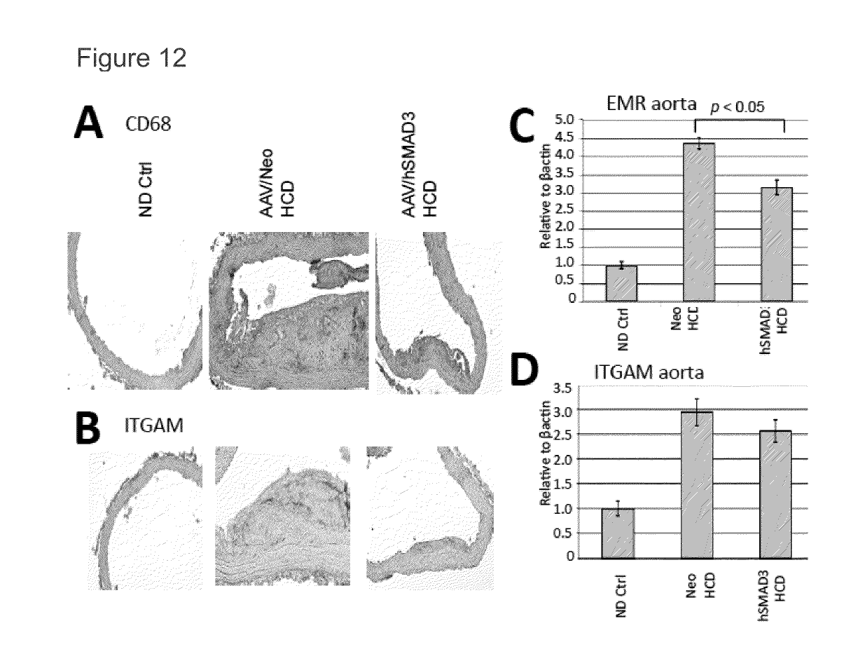

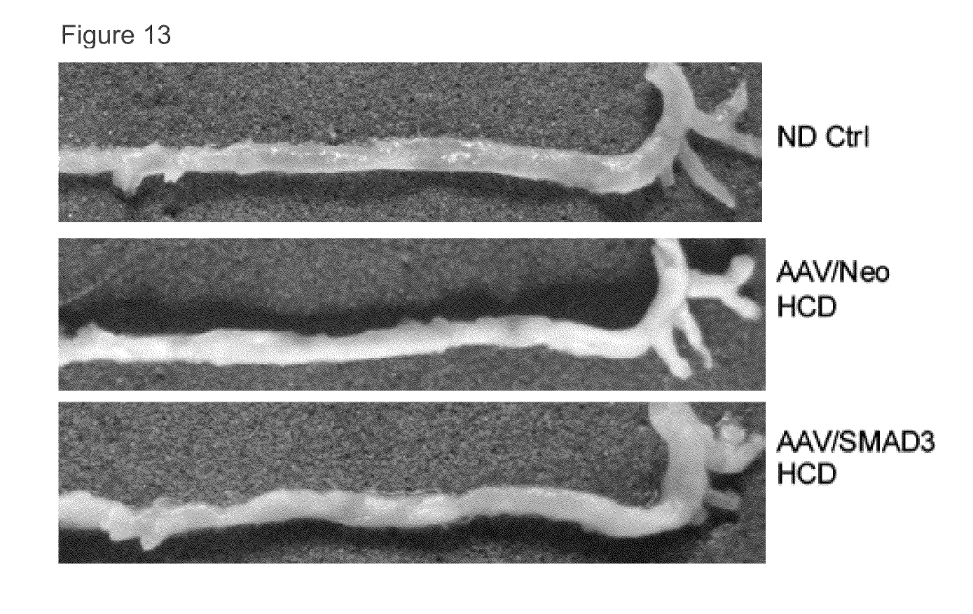

The level of macrophage trafficking into the aortic wall was analyzed by immune-histochemistry using anti-CD68 as shown in FIGS. 12A and 12B, using anti-ITGAM antibody. For both macrophage markers, it is clear that there is a greater number of macrophages in the walls of the Neo/HCD-treated than the SMAD3/HCD-treated animals. Macrophage invasion of the aorta was also quantified by QRT-PCR for the expression of another macrophage marker, EMR. As shown in FIG. 12C, the level of EMR in the SMAD3/HCD-treated animals was significantly lower (p<0.05) than in Neo/HCD-treated animals. QRT-PCR analysis of ITGAM, as another Mo/Mac marker, also trended lower, consistent with the immune-histochemistry data. Thus, these data, taken together, indicate that macrophage levels are lower SMAD3/HCD-treated animals than in Neo/HCD-treated animals. Representative cleaned and unstained aortas are shown in FIG. 13, showing that the AAV/Neo-HCD-treated aorta had higher levels of lipid accumulation in contrast to either the ND ctrl or to the AAV/SMAD3-HCD-treated aorta. No quantification was performed.

### Immune Status of the Aortas

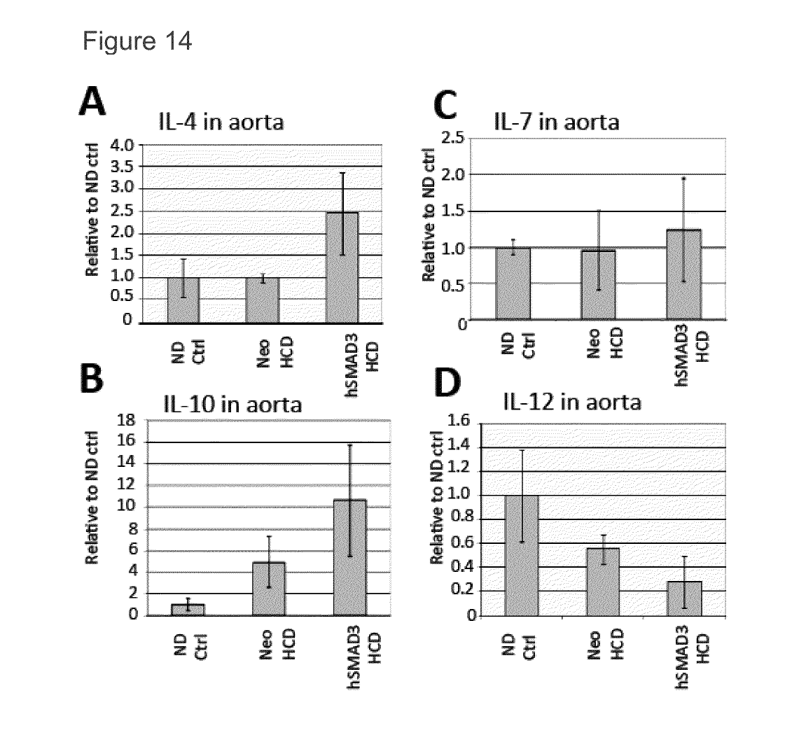

Related to macrophage and lipid accumulation, we observed the expression of various Th1 and Th2 cytokines in the aortas in order to determine the aortas' predominant immune state. FIG. 14A shows that Th2 cytokine IL-4 was significantly (p>0.05) higher in Neo/HCD-treated animals than in the SMAD3/HCD-treated animals. Similarly, IL-10 levels, another Th2 cytokine, trended higher in the SMAD3/HCD-treated animals (FIG. 14B). In contrast, IL-7, a Th1 cytokine, was unchanged in all group (FIG. 14C), while IL-12 (FIG. 14D), yet another Th1 cytokine, trended lower SMAD3/HCD-treated animals over Neo/HCD-treated animals. Thus, overall, these data establish that a predominant Th2 response is present in the aortas as a result of the SMAD3 delivery.

### Analysis of Collagen Expression/Fibrosis.

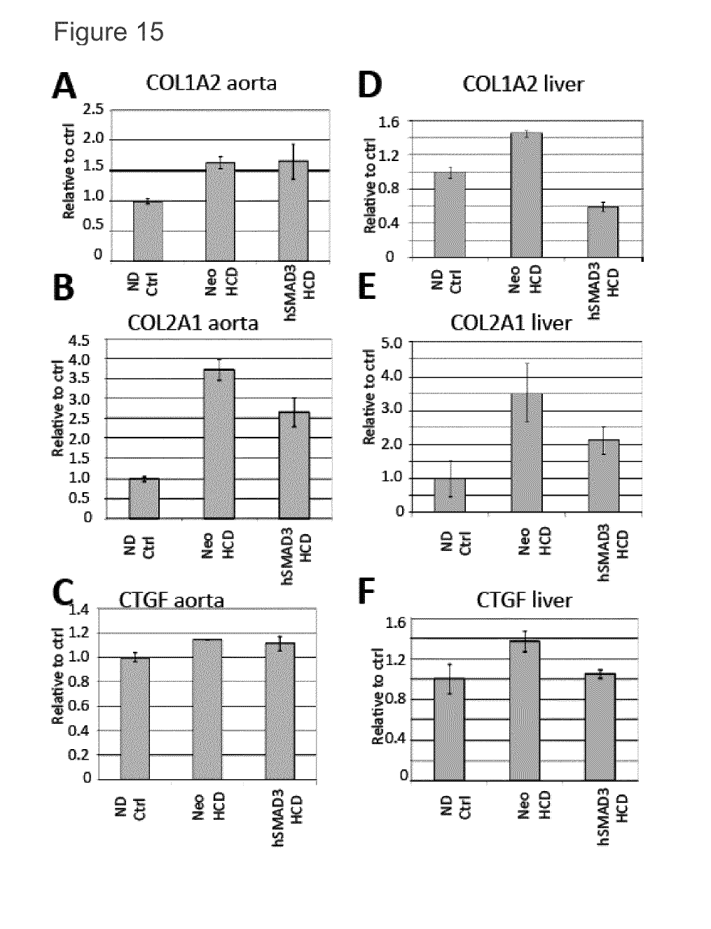

TGFβ1 is very often associated with increased fibrosis, thus it seemed possible that SMAD3 might be similarly associated. In FIG. 15A, the level of collagen 1A expression was analyzed by QRT-PCR and no significant difference in expression in the aortas was found between hSMAD3/HCD- and Neo/HCD-treated animals. However, collagen 2A1 expression (FIG. 15B) was significantly lower in hSMAD3/HCD- than Neo/HCD-treated animals. These data indicate a lack of induction of fibrosis by delivered hSMAD3 expression. Wealso evaluated hepatic collagen 1A expression. It should be noted that the liver is a primary target for AAV8. As shown in FIG. 15C, hepatic collagen 1A expression trended lower in SMAD3/HCD- than in Neo/HCD-treated animals, indicating a lack of induction of fibrosis by higher hSMAD3 expression. The lack of increased fibrosis was further substantiated by observing connective tissue growth factor (CTGF) expression, a known inducer of SMAD3-associated fibrosis, was unchanged throughout all groups (FIG. 15C). In an analogous analysis of the aorta, collagen 1A2, collagen 2A1, and CTGF were all significantly down in AAV/hSMAD3-HDC-treated animals in FIG. 15D-F.

### Example 4

### hFOXP3-AAV Gene Therapy for Cardiovascular Disease

FOXP3 (forkhead box P3) is an anti-inflammatory gene and an important transcription factor of regulatory T cells (Treg). To test whether the FOXP3 gene could be used to limit atherosclerosis, we studied the effects of AAV-based human (h)FOXP3 gene delivery (using AAV2/8 (AAV)/hFOXP3 or AAV/Neo as a control) to inhibit atherogenesis in the low density lipoprotein receptor knockout mice on high-cholesterol diet (LDLR-KO/HCD).

It was found that hFOXP3 gene delivery was associated with significantly lower HCD-induced atherogenesis, as measured by larger aortic lumen cross sectional area, thinner aortic wall thickness, and lower aortic systolic blood velocity compared with Neo gene-HCD-treated controls. Moreover, these measurements taken from the hFOXP3/HCD-treated animals very closely matched those measurements taken from the normal diet (ND) control animals. These data strongly suggest that AAV/hFOXP3 delivery gave a robust anti-atherosclerosis therapeutic effect.

### Materials and Methods

### AAV Vector Construction and Virus Generation

We addressed the hypothesis that hFOXP3 gene delivery can treat or prevent atherosclerosis by using AAV2/8 [AAV2 inverted terminal repeats (ITR) DNA combined with the AAV serotype 8 capsid] gene delivery. The human (h) FOXP3 cDNA was obtained from Open Biosystems or other sources (GenBank Accession number BC113401 or BC143785; or NCBI Reference Sequence NM_001114377) and was ligated downstream from the cytomegalovirus immediate early promoter (CMVpr) within the gutted AAV vector dl3-97 to generate AAV/hFOXP3. The encoded hFOXP3 protein may comprise the amino acid sequence corresponding to GenBank Accession Number: AAI43786 (SEQ ID NO: 16), or AAI13402 (SEQ ID NO: 17). The AAV/Neo vector has been described previously. AAV2/8 virus (AAV2 DNA in AAV8 virion) was produced using pDG8 helper and titered by dot blot analysis by standard methodologies. Zhu et al. (2014) Comparison of efficacy of the disease-specific LOX1- and constitutive cytomegalovirus-promoters in expressing interleukin 10 through adeno-associated virus 2/8 delivery in atherosclerotic mice. PLoS One 9(4):e94665. Zhu et al. (2014) AAV2/8-hSMAD3 gene delivery attenuates aortic atherogenesis, enhances Th2 response without fibrosis, in LDLR-KO mice on high cholesterol diet. J Trans Med 12(1):252. Zhu et al. (2013) Systemic delivery of thiol-specific antioxidant hPRDX6 gene by AAV2/8 inhibits atherogenesis in LDLR KO mice on HCD. Gen. Syndrom Gene Ther. 4(135):2. Cao et al. (2012) Dual AAV/IL-10 plus STAT3 anti-inflammatory gene delivery lowers atherosclerosis in LDLR KO mice, but without increased benefit. Int J Vasc Med 2012:524235. Khan et al. (2011) Systemic hNetrin-1 gene delivery by AAV8 alters leukocyte accumulation and atherogenesis in vivo. Gene Ther 18:437-444. Khan et al. (2010) AAV/hSTAT3-gene delivery lowers aortic inflammatory cell infiltration in LDLR KO mice on high cholesterol. Atherosclerosis 213(1):59-66.

### Animal Treatments

LDLR-KO mice (B6;129S7-Ldlrtm1Her/J) were purchased from Jackson Laboratories (Bar Harbor, Me., USA). Three groups of male mice, composed of ten animals each at 8 weeks old, were injected with AAV/Neo (positive control group), or AAV/hFOXP3 virus at a titer of 1×10¹⁰ e.g./ml via tail vein with 200 μL virus per mouse, two booster injections were followed at an interval of 5-6 days. High cholesterol diet (HCD) of 4% cholesterol and 10% cocoa butter diet (Harlan Teklad, Madison, Wis., USA) was provided from the first day of injection and maintained for the entire study period. Another group of mice fed with a ND was used as a negative control group. The normal background mouse chow was Harlan catalog #7012, and the HCD was #7012, 4% cholesterol/10% cocoa butter, custom formulated by Harlan.

### Ultrasound Imaging

Ultrasound imaging was carried out using a Vevo 770 High-Resolution Imaging system (Visualsonics, Toronto, Canada) with a RMV 707B transducer having a center frequency of 30 MHz. Animal preparation was done as described earlier. Martin-McNulty et al. (2005) Noninvasive measurement of abdominal aortic aneurysms in intact mice by a high-frequency ultrasound imaging system. Ultrasound Med Biol 31(6):745-749. In brief, the mice were anesthetized using 1.5% isoflurane (Isothesia, Abbott Laboratories, Chicago, USA) with oxygen and laid supine out on a thermostatically heated platform. Abdominal hair was removed with a shaver and a chemical hair remover (Church & Dwight Co, Inc., NJ, USA). A pre-warmed transducing gel (Medline Industries, Inc., Mundelein, USA) was spread over the skin as a coupling medium for more accurate measurements. Two general levels of the vessel were visualized: thoracic region—below the aortic arches to the diaphragm and then the renal region—the upper abdominal region to the iliac bifurcation. Image acquisition was started on B-mode, where, a long axis view was used to visualize the length of the aorta. Then the scan head probe was turned 90° for a short-axis view to visualize the cross-sectional area of the aorta. Individual frames and cine loops (300 frames) were acquired at all levels of the aorta, and included both the long axis and short axis view and recorded at distances of 1 mm throughout the length of the aorta. Measurement of the flow velocity, orientation of the abdominal aorta by ultrasound, was accomplished by tilting the platform and the head of mouse down with the transducer probe towards the feet and tail of the mouse. This positioning resulted in the Doppler angle to be less than 60° for accurate measurements of blood flow velocity in the pulse-wave Doppler (PW) mode within abdominal aorta. Off-line measurements and data analysis was performed using the customized version of Vevo770 Analytical Software from both the longitudinal and transverse images. The complete imaging for each mouse lasted for about 25-30 min.

### Measurement of Plasma Cholesterol

Total plasma cholesterol of AAV/Neo and AAV/FOXP3 mice were measured by VetScan VS2 (Abaxis, Union City, Calif., USA) at the Veterans Animal Laboratory (VAMU).

### Atherosclerotic Lesion Analysis by Direct Visualization

Whole dissected aortas were fixed in 10% buffered formalin, inspected under a dissecting microscope and any small pieces of adventitial fat that remained attached were removed very carefully without disturbing the aorta itself and the internal lipid accumulations/plaque. Unstained small animal aortas are normally translucent but show lipid deposition as white areas. Daugherty et al. (2003) Quantification of atherosclerosis in mice. Mol Biol 209(293-309):466. Chen et al. (2002) In vivo imaging of proteolytic activity in atherosclerosis. Circulation 105:2766-2771. Aortas were then photographed under natural light using a 10 megapixel digital camera (Nikon, Japan).

### Observation of Atherosclerosis by Histology

Twenty weeks after first injection of virus and on HCD, mice were killed by CO2 exposure. Entire aortas, including the aortic arches, thoracic and abdominal aortas, were removed. The aorta was flushed with saline solution and fixed in 10% neutral buffered formalin (Sigma). After 24 h, the fixed tissue was used for paraffin embedding and sectioning for histological analysis. Finally, representative sections were hematoxylin and eosin-stained.

### Statistics

Parameters were analyzed with statistics software SPSS 16.0 by nonparametric ANOVA test. If differences were detected between means, Newman-Keuls test was used for multiple comparisons. Difference were considered as significant if P<0.05.

### Results

### AAV Vectors and Animal Treatments

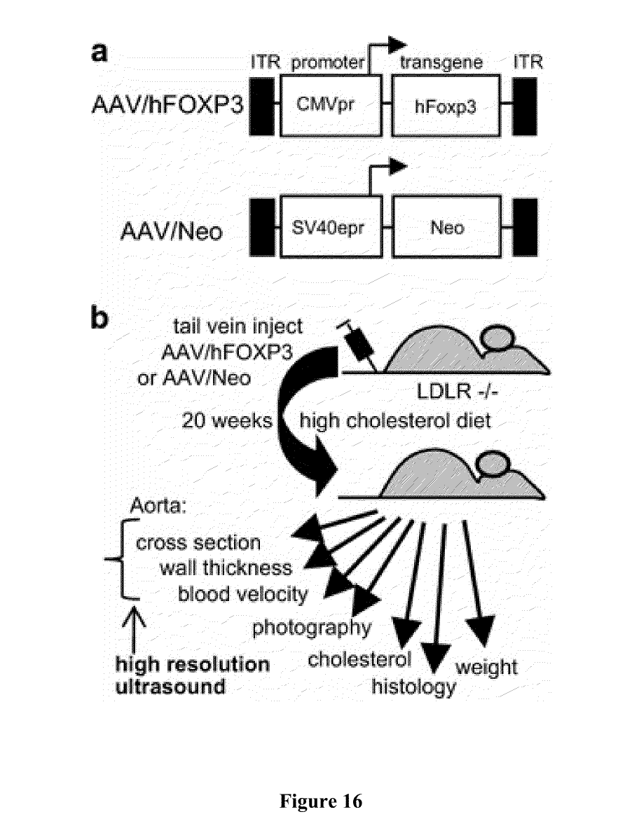

We addressed the hypothesis that hFOXP3 gene delivery can inhibit atherosclerosis by using AAV2/8 [AAV2 inverted terminal repeats (ITR) DNA combined with the AAV serotype 8 capsid] gene delivery. The AAV2/8-hFOXP3 was delivered by tail vein injection and the animals placed on HCD (4% cholesterol, 10% cocoa butter). Another animal group received AAV2/8-Neo virus delivered by tail vein injection and also placed on HCD, which served as positive controls. Lastly, a group of animals received only a ND and served as negative controls. The animals were then analyzed by high resolution ultrasound and harvested at 20 weeks post-injection/post HCD initiation. FIG. 16A shows the general structure of the AAV vectors used. FIG. 16B shows the overall structure of the experiments. We have previously demonstrated specific delivery and expression of multiple transgenes into the aorta by AAV2/8 (AAV2 DNA inside the AAV8 capsid), utilizing the CMVpr transcriptional promoter, delivered by tail vein injection, to give expression levels 1.7-2.3% that of β-actin. Zhu et al. (2014) AAV2/8-hSMAD3 gene delivery attenuates aortic atherogenesis, enhances Th2 response without fibrosis, in LDLR-KO mice on high cholesterol diet. J Trans Med 12(1):252. Zhu et al. (2013) Systemic delivery of thiol-specific antioxidant hPRDX6 gene by AAV2/8 inhibits atherogenesis in LDLR KO mice on HCD. Gen. Syndrom Gene Ther. 4(135):2.

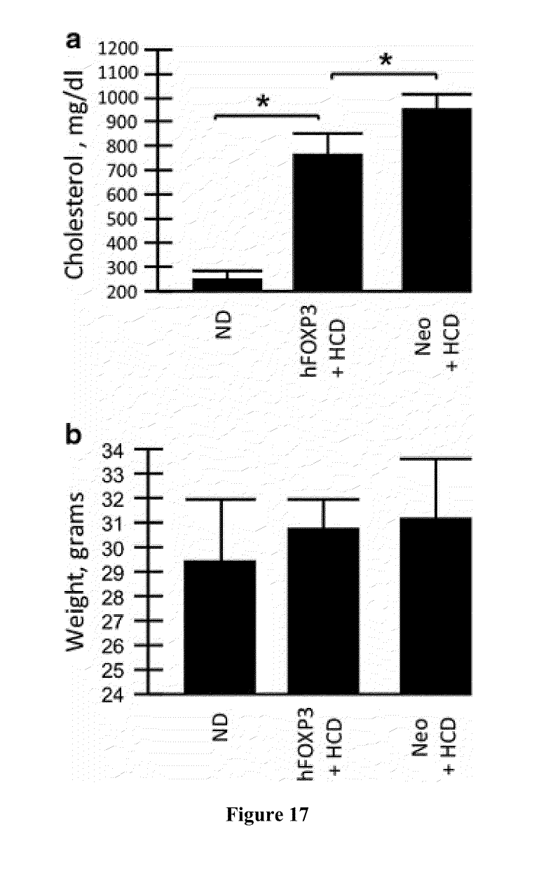

FIG. 17A shows that the blood cholesterol levels were high in both groups on HCD compared to the ND control. However, the AAV/hFOXP3-HCD and AAV/Neo-HCD treated animal were statistically different, with the AAV/ hFOXP3-HCD group having a lower blood cholesterol level (e.g., about 20%, at least 15%, or at least 20% lower than the control AAV/Neo-HCD treated animal). In FIG. 17B, animal weights were statistically similar in all groups.

### Analysis of Aortic Structure

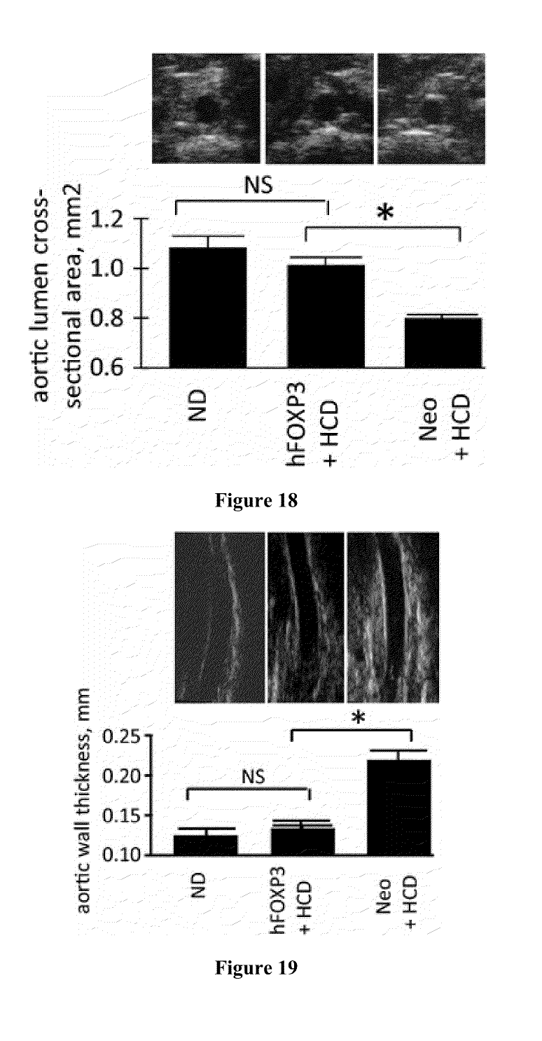

High resolution ultrasound (HRUS) was then used to analyze the aortas of at least eight animals per group. FIG. 18 shows that the cross-sectional area of the lumens of the aortas was significantly larger (p<0.05) in the hFOXP3/HCD-treated animals (e.g., about 25%, at least 15%, at least 20% or at least 25% greater) than the Neo/HCDtreated animals by HRUS. Moreover, the lumens of the aortas in the hFOXP3/HCD-treated animals versus the ND control animals (negative control) were statistically the same (not significant, NS). This evidence of robust efficacy was surprising to us as Netrinl gene delivery, while showing efficacy, did not give this higher level of efficacy.

Moreover, further HRUS analysis, as shown in FIG. 19, indicated that aortic wall thickness was significantly thinner (p<0.05) in the hFOXP3/HCD-treated animals (e.g., about 40%, at least 20%, at least 25%, at least 30%, or at least 40% thinner) than the Neo/HCD-treated, positive control animals. Additionally, the wall thickness of the aorta in the hFOXP3/HCD-treated animals and the ND control animals were statistically the same (not significant, NS). Thus, this additional evidence of robust efficacy fully supports the lumen area analysis.

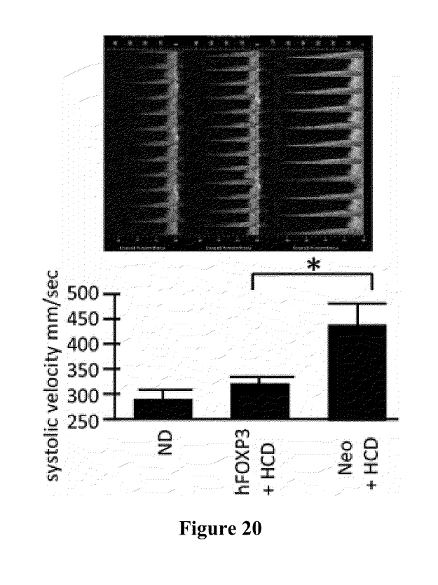

FIG. 20 shows that the systolic blood velocity (systolic pulse wave velocity) in the aorta of the hFOXP3/HCD-treated animals was significantly lower (p<0.05) (e.g., about 25%, at least 15%, at least 20% or at least 25% lower) than the Neo/HCD-treated animals, again consistent with a lower level of atherosclerosis. In this additional measurement the wall thickness of the aorta in the hFOXP3/HCD-treated animals and the ND control animals were statistically significant.

However, it can also be easily observed that the systolic blood velocity of the hFOXP3/HCD-treated animals was much closer (lower) to the ND animals than to the Neo/HCD-treated animals. The correlation between low systolic blood velocity with thinner aortic wall thickness and larger aortic lumens we observed here matches the correlations we have seen in our other anti-atherosclerosis gene delivery studies in our studies of LDLR-KO mice on 4% cholesterol, 10% cocoa butter HCD. This again indicated that the hFOXP3 gene delivery did provide high level of efficacy against HCD-associated atherosclerosis.

### Visual Inspection of Aortas

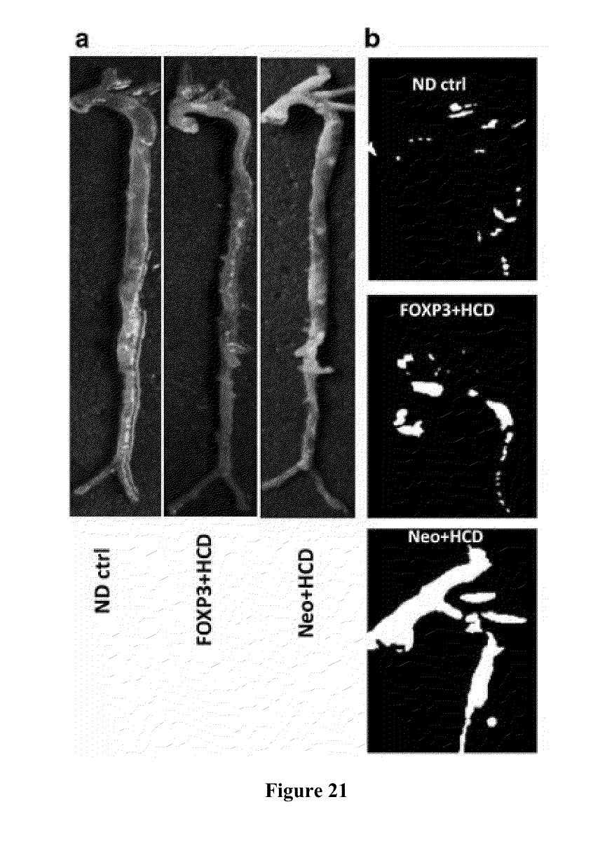

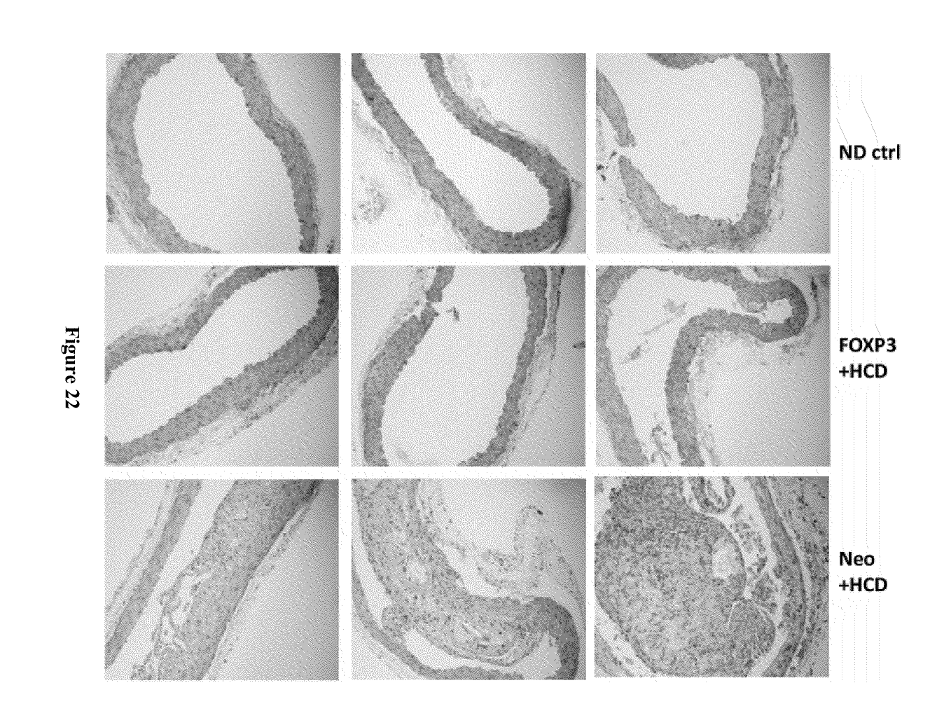

We further studied the effects of the transgene hFOXP3 transgene on the aorta challenged with HCD by unstained small animal aortas show lipid deposition as white areas. FIG. 21A shows representative unstained aortas from the indicated animal groups. It is readily visible that the AAV/Neo-HCD-treated animals have much more numerous areas of white in both the aortic arch and in the descending aorta than either the ND or AAV/hFOXP3-HCD-treated animals. As the aortic arch is particularly susceptible to atherosclerosis the aortic arches in these same aortas were further image enhanced using MS PowerPoint black-white 25% display as shown in FIG. 21B. Again, consistent with the HRUS measurements, the AAV/hFOXP3-HCD-treated animal showed much less atherosclerosis than the AAV/Neo-HCD-treated animal, and was very similar to the ND animal.

### Histologic Views of Representative Aortas

Finally, histologic sections were taken across the axis of the aortas to give representative cross sectional views. In FIG. 22, representative hematoxylin and eosin-stained sections are shown, one each from each of three animals, from each of the three animal treatment groups. Note that the AAV/Neo-HCD group showed much higher levels of atherosclerotic plaque than either the ND or AAV/FOXP3-HCD group, consistent with the HRUS (FIGS. 18, 19 and 20) and the direct visualization (FIG. 21) analysis. The photomicrographs were further analyzed for smooth muscle layer thickness, but no statistical significance was seen. However, within the AAV/Neo-HCD animal group an increase in smooth muscle layer thickness of 61% (p<0.05) was observed in regions associated with plaque compared to the opposing non-atherosclerotic region of the same micrograph.

### Discussion

This study has demonstrated that CMVpr-FOXP3-gene delivery by AAV2/8 vector results in a robust protection of aortas from developing atherosclerosis in the LDLR-KO/HCD model. The protection afforded by the FOXP3-treated animals on HCD was shown by the finding that multiple aortic parameters were not statistically different from the ND control group. These data suggest that the FOXP3 gene gives a high level of efficacy against HCD-induced atherosclerosis in the well-established LDLR/KO-HCD animal model.

It is known that the loss of FOXP3 expression (or mutation FOXP3) results in increases in chronic autoimmunity. Ziegler S F (2006) FOXP3: of mice and men. Annu Rev Immunol. 24:209-226. The phenotype of FOXP3 knockouts gives the “scurfy” phenotype in mice and in humans generates the X-linked autoimmunity-allergic dysregulation and immuno-dysregulation, X-linked syndromes. Brunkow et al. (2001) Disruption of a new forkhead/wingedhelix protein, scurfin, results in the fatal lymphoproliferative disorder of the scurfy mouse. Nat Genet. 27:68-73. Bennett et al. (2001) The immune dysregulation, polyendocrinopathy, enteropathy, X-linked syndrome (IPEX) is caused by mutations of FOXP3. Nat. Genet 27:20-21. Tregs are also important for lowering excess inflammation and giving tolerance to gut commensal microbes. Littman et al. (2010) Th17 and regulatory T cells in mediating and restraining inflammation. Cell 140:845-858. However, if present in extreme excess, Tregs may allow for dysplastic and malignant cell growth and chronic infections through the governance of limited anti-tumor surveillance or limited anti-pathogenic organism immune responses. Zou W (2006) Regulatory T cells, tumour immunity and immunotherapy. Nat Rev Immunol 6:295-307. Workman et al. (2009) The development and function of regulatory T cells. Cell Mol Life Sci 66:2603-2622. Fontenot et al. (2003) Foxp3 programs the development and function of CD4+CD25+regulatory T cells. Nat. Immunol. 4:330-336. Williams et al. (2007) Maitenance of the Foxp3-dependent developmental program in mature regulatory T cells requires continued expression of Foxp3. Nat Immunol. 8:277-284.

The use of AAV-based FOXP3 gene therapy showed efficacy against inflammation and cardiovascular diseases.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

### CITATIONS

1) Hermonat P L, and Muzyczka N. Use of adeno-associated virus as a mammalian DNA cloning vector: transduction of neomycin resistance into mammalian tissue culture cells. Proc. Natl. Acad. Sci. U.S.A. 81:6466-6470, 1984.

2) Hermonat P L. The first adeno-associated virus gene transfer experiment, 1983. Human Gene Therapy, 2014, 25(6):486-7

3) Buchlis, G, Podsakoff G M, Radu A, Hawk S M, Flake A W, Mingozzi F, High K A. Factor IX expression in skeletal muscle of a severe hemophilia B patient 10 years after AAV-mediated gene transfer. Blood, 2012 Mar. 29; 119(13):3038-41.

4) Zhu H, Cao M, Chriva-Internati M, Hermonat P L. Comparison of efficacy of human interleukin 10, expressed from the disease-specific LOX1 or constitutive cytomegalovirus promoters, against atherosclerosis in mice using adeno-associated virus 2/8 delivery. in press PLoS ONE, 2014.

5) Zhu H, Cao M, Figueroa J A, Cobos E, Uretsky B F, Chiriva-Internati M, Hermonat, P L. (2014) AAV2/8-hSMAD3 gene delivery attenuates aortic atherogenesis, enhances Th2 response, without inducing COL1A2/2A1 and CTGF (fibrosis) in LDLR-KO mice on high cholesterol diet. In press J Translational Medicine.

6) Zhu H, Cao M, Straub K D, Hermonat P L. (2013) Systemic delivery of thiol-specific antioxidant hPRDX6 gene by AAV2/8 inhibits atherogenesis in LDLR KO mice on HCD. In press Genetic Syndromes and Gene Therapy

7)Damdindorj L, Kaman S, Ota A, Hossain E, Konishi Y, Hosokawa Y, Konishi H. A comparative analysis of constitutive promoters located in adeno-associated viral vectors. PLoS One. 2014 Aug. 29; 9(8):e106472

8) Filippi C M, von Herrath M G. IL-10 and the resolution of infections. High levels of IL10 are associated with increased viral, bacterial, and fungal infections, as well as cancer. J Pathol. 214: 224-230, 2008.

9) Zobel, Katrin. Martus, Peter. Pletz, Mathias W. Ewig, Santiago. Prediger, Michael. Welte, Tobias. Buhling, Frank. Interleukin 6, lipopolysaccharide-binding protein and interleukin 10 in the prediction of risk and etiologic patterns in patients with community-acquired pneumonia: results from the German competence network CAPNETZ. BMC Pulmonary Medicine. 12:6, 2012.

10) Clemons K V. Grunig G. Sobel R A. Mirels L F. Rennick D M. Stevens D A. Role of IL-10 in invasive aspergillosis: increased resistance of IL-10 gene knockout mice to lethal systemic aspergillosis. Clinical & Experimental Immunology. 122(2):186-91, 2000.

11) Brooks, D. G., A. M. Lee, H. Elsaesser, D. B. McGavern, and M. B. Oldstone. IL-10 blockade facilitates DNA vaccine-induced T cell responses and enhances clearance of persistent virus infection. J. Exp. Med. 205:533-541, 2008.

12) Brooks, D. G., M. J. Trifilo, K. H. Edelmann, L. Teyton, D. B. McGavern, and M. B. Oldstone. Interleukin-10 determines viral clearance or persistence in vivo. Nat. Med. 12:1301-1309, 2006.

13) Ejrnaes, M., C. M. Filippi, M. M. Martinic, E. M. Ling, L. M. Togher, S. Crotty, and M. G. von Herrath. Resolution of a chronic viral infection after interleukin-10 receptor blockade. J. Exp. Med. 203:2461-2472, 2006.

14) Zeni E, Mazzetti L, Miotto D, Lo Cascio N, Maestrelli P, et al. Macrophage expression of interleukin-10 is a prognostic factoir in nonsmall cell lung cancer. Eur Respir J. 30: 627-632, 2007.

15) Maris, C. H., C. P. Chappell, and J. Jacob. Interleukin-10 plays an early role in generating virus-specific T cell anergy. BMC Immunol. 8:8. 2007.

16) Herfarth H, Scholmerich J. IL-10 therapy in Crohn's disease: at the crossroads. Gut 50:146-147.

17) Hermonat P L, Zhou H, Cao M, Mehta J L. LOX-1 transcription. Cardiovasc Drugs Ther., 2012.

18) Flotte T R, Afione S A, Solow R, Drumm M L, Markakis D, Guggino W B, Zeitlin P L, Carter B J. Expression of the cystic fibrosis transmembrane conductance regulator from a novel adeno-associated virus promoter. J Biol Chem. 268: 3781-3790, 1993.

19) Haberman R P, McCown T J, Samulski R J. Novel transcriptional regulatory signals in the adeno-associated virus terminal repeat A/D junction element. J Virol 74: 8732-8739, 2000.

20) Libby, P, Ridker, P M, Maseri, A. (2002) Inflammation and Atherosclerosis. Circulation. 105: 1135-1143.

21) Libby, P, Ridker, P M, Hansson, G K. Inflammation in atherosclerosis. (2009) J Amer Coll Card 54:2129-2138.

22) Ludewig B, Laman J D. (2004) The in and out of monocytes in atherosclerotic plaques: Balancing inflammation through migration. Proc Natl Acad Sci USA. 101: 11529-11530.

23) Woollard K J, Geissmann F. (2010) Monocytes in atherosclerosis: subsets and functions. Nat Rev Cardiol. 7: 77-86.

24) Lacolley P, Regnault V, Nicoletti A, Li Z, Michel J B. The vascular smooth muscle cell in arterial pathology: a cell that can take on multiple roles. Cardiovasc Res 95: 194-2004, 2012.

25) Rebsen S S M, Doevendans P A F M, van Eys G J J M. Regulation and characteristics of vascular smooth muscle cell phenotypic diversity. Neth Heart J. 15: 100-108, 2007.

26) Liu Y, Li D, Chen J, Xie J, Bandyopadhyay S, et al. (2006) Inhibition of atherogenesis in LDLR knockout mice by systemic delivery of adeno-associated virus type 2-hIL-10. Atherosclerosis 188: 19-27.

27) Cao M, Khan J A, Kang B Y, Liu Y, Mehta J L, Hermonat P L. Dual AAV/IL-10 plus STAT3 anti-inflammatory gene delivery lowers atherosclerosis in LDLR KO mice, but without increased benefit. Int J Vasc Med. 2012: 52435, 2012.

28) Chen S, Kapturczak M H, Wasserfall C, Glushakova O Y, Campbell-Thompson M, et al. (2005) Interleukin 10 attenuates neointimal proliferation and inflammation in aortic allografts by a heme oxygenase-dependent pathway. Proc Natl Acad Sci 102: 7251-7256.

29) Yoshioka T, Okada T, Maeda Y, Ikeda U, Shimpo M, et al. (2004) Adeno-associated virus vector-mediated interleukin-10 gene transfer inhibits atherosclerosis in apolipoprotein E-deficient mice. Gene Therapy 11: 1772-1779.

30) Sun J, Li X, Feng H, Gu H, Blair T, Li J, Soriano S, Meng Y, Zhang F, Feng Q, Yang X. Magnetic resonance imaging of bone marrow-cell mediated interleukin-10 gene therapy of atherosclerosis. PloS One 6(9): e24529, 2011

31) Han X, Kitamoto S, Wang H, Boisvert WA. Interleukin-10 overexpression in macrophages suppresses atherosclerosis in hyperlipidemic mice. FASEB J 24: 2869-2880, 2010.

32) Li, D., et al. Suppression of Atherogenesis by Delivery of TGF [symbol] 1ACT Using Adeno-Associated Virus Type 2 in LDLR Knockout Mice. Biochem. Biophys. Res. Commun. 344: 701-707, 2006.

33) Dandapat A, Hermonat P L, Mehta J L. Over-expression of TGFbeta(1) by adeno-associated virus type 2 vector protects myocardium from ischemia-reperfusion injury. Gene Ther. 15: 415-23, 2008.

34) Letterio J J, Roberts A B. (1998) Regulation of immune responses by TGF-beta. Annu Rev Immunol 16:137-161.

35) de Vries J E. (1995) Immunosuppressive and anti-inflammatory properties of interleukin 10. Ann Med. 27:537-41.

36) Byfield S D, Roberts A B. (2004) Lateral signaling enhances TGF-beta response complexity. Trends Cell Biol 14:107-111.

37) Attisano L, Wrana J L. (2002) Signal Transduction by the TGF-beta Superfamily. Science 296:1646-1647.

38) Kopp J B, Factor V M, Mozes M, Nagy P, Sanderson N, et al. (1996) Transgenic mice with increased plasma levels of TGF-beta 1 develop progressive renal disease. Lab Invest 74: 991-1003.

39) Gressner A M, Weiskirchen R, Breitkopf K, Dooley S. (2002) Roles of TGF-beta in hepatic fibrosis. Front Biosci 7: 793-807.

40) Filippi C M, von Herrath M G. IL-10 and the resolution of infections. High levels of IL10 are associated with increased viral, bacterial, and fungal infections, as well as cancer. J Pathol 214: 224-230, 2008.

41) Zobel, Katrin. Martus, Peter. Pletz, Mathias W. Ewig, Santiago. Prediger, Michael. Welte, Tobias. Buhling, Frank. Interleukin 6, lipopolysaccharide-binding protein and interleukin 10 in the prediction of risk and etiologic patterns in patients with community-acquired pneumonia: results from the German competence network CAPNETZ. BMC Pulmonary Medicine. 12:6, 2012.

42) Clemons K V. Grunig G. Sobel R A. Mirels L F. Rennick D M. Stevens D A. Role of IL-10 in invasive aspergillosis: increased resistance of IL-10 gene knockout mice to lethal systemic aspergillosis. Clinical & Experimental Immunology. 122(2):186-91, 2000 November.

43) Brooks, D. G., A. M. Lee, H. Elsaesser, D. B. McGavern, and M. B. Oldstone. 2008. IL-10 blockade facilitates DNA vaccine-induced T cell responses and enhances clearance of persistent virus infection. J. Exp. Med. 205:533-541.

44) Brooks, D. G., M. J. Trifilo, K. H. Edelmann, L. Teyton, D. B. McGavern, and M. B. Oldstone. 2006. Interleukin-10 determines viral clearance or persistence in vivo. Nat. Med. 12:1301-1309.

45) Ejrnaes, M., C. M. Filippi, M. M. Martinic, E. M. Ling, L. M. Togher, S. Crotty, and M. G. von Herrath. 2006. Resolution of a chronic viral infection after interleukin-10 receptor blockade. J. Exp. Med. 203:2461-2472.

46) Maris, C. H., C. P. Chappell, and J. Jacob. 2007. Interleukin-10 plays an early role in generating virus-specific T cell anergy. BMC Immunol. 8:8.

47) Herfarth H, Scholmerich J. (2002) IL-10 therapy in Crohn's disease: at the crossroads Gut 50:146-147.

48) Asadullah K, Sterry W, Volk H D. (2003) Interleukin-10 Therapy—Review of a New Approach Pharmacological Reviews 55: 241-269.

49) Hermonat P L, Muzyczka N. (1984) Use of adeno-associated virus as a mammalian DNA cloning vector: transduction of neomycin resistance into mammalian tissue culture cells. Proc Natl Acad Sci USA 81: 6466-6470.

50) Tratschin J D, West M H, Sandbank T, Carter B J. (1984) A human parvovirus, adeno-associated virus, as a eukaryotic vector: transient expression and encapsidation of the prokaryotic gene for chloramphenicol acetyltransferase, Molec Cell Biol 4: 2072-2081.

51) Hermonat P, Labow, L M, R. Wright, Berns K I, Muzyczka N. (1984) Genetics of adeno-associated virus: isolation and preliminary characterization of mutants in adeno-associated virus type 2. J Virol 51: 329-339.

52) Liu Y, Chiriva-Internati M, Grizzi F, Salati E, Roman J J, Lim S, et al. (2001) Rapid induction of cytotoxic T cell response against cervical cancer cells by human papillomavirus type 16 E6 antigen gene delivery into human dendritic cells by an adeno-associated virus vector. Can Gene Ther 8: 948-957.

53) Chiriva-Internati M, Liu Y, Weidanz J A, Grizzi F, You H, Zhou W, et al. (2003) Testing recombinant adeno-associated virus-gene loading of dendritic cells for generating potent cytotoxic T lymphocytes against a prototype self-antigen, multiple myeloma HM1.24. Blood. 102: 3100-3107.

54) You C X, Shi M, Liu Y, Cao M, Luo R C, et al. (2012) AAV2/IL-12 gene delivery into dendritic cells (DC) enhances CTL stimulation above other IL-12 applications: evidence for IL-12 intracrine activity in DC. Oncoimm 2012: 1:847-855.

55) Jiang H., Pierce G F, Ozelo M C, de Paula E V, Vargas J A, et al. (2006) Evidence of multiyear factor IX expression by AAV-mediated gene transfer to skeletal muscle in an individual with severe hemophilia B. Molec Ther 14: 452-455.

56) Wettergren E E, Gussing F, Quintino L, Lundberg C. (2012) Novel disease-specific promoters for use in gene therapy for Parkinson's disease. Neurosci Lett 530: 29-34

57) Kim H A, Mahato R I, Lee M. (2009) Hypoxia-specific gene expression for ischemic disease gene therapy Adv Drug Del Rev 61: 614-622.

58) Sawamura T, Kume N, Aoyama T, Morlaki H, Hoshikawa H, et al. (1997) An endothelial receptor for oxidized low-density lipoprotein. Nature 386:73-77.

59) Oka K, Sawamura T, Kikuta K, Itokawa S, Kume N, et al. (1998) Lectin-like oxidized low density lipoprotein receptor 1 mediates phagocytosis of aged/apoptotic cells in endothelial cells. Proc Natl Acad Sci USA 95: 9535-9540.

60) Mehta J L, Li D. (1998) Identification and autoregulation of receptor for OX-LDL in cultured human coronary artery endothelial cells. Biochem Biophys Res Commun 248: 511-514.

61) Kakutani M, Masaki T, Sawamura T. (2000) A platelet-endothelium interaction mediated by lectin-like oxidized low-density lipoprotein receptor-1. Proc Natl Acad Sci USA. 97: 360-364.

62) Ogura S, Kakino A, Sato Y, Fujita Y, Iwamoto S, Otsui K, et al. (2009) LOX-1: the multifunctional receptor underlying cardiovascular dysfunction. Circ J 73:1993-1999.

63) Aoyama T, Sawamura T, Furutani Y, Matsuoka R, Yoshida MC, Fujiwara H, et al. (1999) Structure and chromosomal assignment of the human lectin-like oxidized low-density-lipoprotein receptor-1 (LOX-1) gene. Biochem J. 339:177-184.

64) Khan, J A, Cao M, Kang B Y, Liu Y, Mehta J L, Hermonat P L. (2010) AAV/hSTAT3-gene delivery lowers aortic inflammatory cell infiltration in LDLR KO mice on high cholesterol diet. Atherosclerosis 213: 59-66.

65) Richter M, Iwata A, Nyhuis J, Nitta Y, Miller A D, Halbert C L, Allen M D. Adeno-associated virus vector transduction of vascular smooth muscle cells in vivo. Physiol Genomics 2: 117-127, 2000.

66) Liu Q, Muruve D A. Molecular basis of the inflammatory response to adenovirus vectors. Gene Ther 10: 935-940, 2003.

67) Jooss K, Chirmule N. Immunity to adenovirus and adeno-associated viral vectors: implications for gene therapy. Gene Ther 10: 955-963, 2003.

68) Work L M, Nicklin S A, Brain N J R, Dishart K L, von Seggern D J, et al. Development of efficient viral vectors selective for vascular smooth muscle cells. Mol Ther 9: 198-208, 2004.

69) Zhong L, Li B, Mah C S, Govindasamy L, Agbandje-McKenna M, Cooper M, et al. Next generation of adeno-associated virus vectors: point mutations in tyrosines lead to high-efficiency transduction at lower doses. Proc Natl Acad Sci USA 105: 7827-7832, 2008.

70) Wang Z, Zhu T, Qiao C, Zhou L, Wang B, et al. Adeno-associated virus serotype 8 efficiently delivers genes to muscle and heart. Nat Biotechnol 23: 321-328, 2005.

71) Efficacy and safety of adeno-associated viral vectors based on serotype 8 and 9 vs lentiviral vectors for hemophilia B gene therapy. Vandendriessche T, Thorrez L, Acosta-Sanchez A, Petrus I, Wang L, et al. Efficacy and safety of adeno-associated viral vectors based on serotype 8 and 9 vs lentiviral vectors for hemophilia B gene therapy. J Thromb Haemost 5: 16-24, 2007.

72) Hermonat P L, Quirk J G, Bishop B M, Han L. The Packaging capacity of adeno-associated virus and the potential for wild type-plus AAV gene therapy vectors. FEBS Letters 407:78-84, 1997.

73) Hofnagel O, Luechtenborg B, Stolle K, Lorkowski S, Eschert H, Plenz G, Robenek H. Proinflammatory cytokines regulate LOX-1 expression in vascular smooth muscle cells. Arterioscler Tromb Vasc Biol 24: 1789-1795, 2004.

74) Sun Y, Chen X. Ox-LDL-induced LOX-1 expression in vascular smooth muscle cells: role of reactive oxygen species. Fundam Clin Pharmacol 25: 572-579, 2011.

75) Michelfelder S, Varadi K, Raupp C, Hunger A, Korbelin J, Pahrmann C, et al. Peptide ligands incorporated into the threefold spike capsid domain to re-direct gene transduction of AAV8 and AAV9 in vivo. PlosOne 6: e23101, 2011.

76) Kay C N, Ryals R C, Aslanidi G V, Min S H, Ruan Q, Sun J, et al. Targeting photoreceptors via intravitreal delivery using novel capsid-mutated AAV vectors. PlosOne 8: e62097, 2013.

77) Shiomi M, Koike T, Ito T. Contribution of the WHHL rabbit, an animal model of familial hypercholesterolemia, to elucidation of the anti-atherosclerotic effects of statins. Atherosclerosis 231: 39-47, 2013.

78) Liu Y, Li D, Chen J, Xie J, Bandyopadhyay S, et al. (2006) Inhibition of atherogenesis in LDLR knockout mice by systemic delivery of adeno-associated virus type 2-hIL-10. Atherosclerosis 188: 19-27.

79) Chen J, Liu Y, Liu H, Hermonat P L, Mehta J L. (2006) Lectin-like oxidized low-density lipoprotein receptor-1 (LOX-1) transcriptional regulation by Oct-1 in human endothelial cells: implications for atherosclerosis. Biochemical J 393: 255-265.

80) Chen J, Liu Y, Liu H, Hermonat P L, Mehta J L. (2006) Molecular dissection of angiotensin II-activated human LOX-1 promoter. Arterioscler Thromb Vasc Biol. 26:1163-1168.

81) Qin J Y, Zhang L, Clift K L, Hulur I, Xiang A P, et al. (2010) Systematic comparison of constitutive promoters and the doxycyline-inducible promoter. PLOSone 5: e10611.

82) Zarrin A A, Malkin L, Fong I, Luk K D, Ghose A, et al. (1999) Comparison of CMV, RSV, SV40 viral and V lambda 1 cellular promoters in B and T lymphoid and non-lymphoid cell lines. Biochim Biophys Acta 1446: 135-139.

83) Faber B C, Cleutjens K B, Niessen R L, Aarts P L, Boon W, et al. (2001) Identification of genes potentially involved in rupture of human atherosclerotic plaques. Circ Res 89: 547-554.

84) Armstrong P J, Johanning J M, Calton Jr W C, Delatore J R, Franklin D P, et al. (2002) Differential gene expression in human abdominal aorta: aneurysmal versus occlusive disease. J Vasc Surg 35: 346-355.

85) Hiltunen M O, TuomistoBrasen J H, Rissanen T T, et al. (2002) Changes in gene expression in atherosclerotic plaques analyzed using DNA array. Atherosclerosis 165: 23-32.

86) Martinet W, Schrijvers D M, De Meyer G R, Thielemans J, Knaapen M W, et al. (2002) Gene expression profiling of apoptosis-related genes in human atherosclerosis: upregulation of death-associated protein kinase. Arterioscler Thromb Vasc Biol 22: 2023-2029.

87) Woodside K J, Hernandez A, Smith F W, Xue X Y, Hu M, et al. (2003) Differential gene expression in primary and recurrent carotid stenosis. Biochem Biophys Res Commun 302: 509-514.

88) Stary H C. (2000) Natural History and Histological Classification of Atherosclerotic Lesions: An Update, Arterioscler Thromb Vasc Biol 20: 1177-1178.

89) Stone G W, Maehara A, Lansky A J, de Bruyne B, Cristea E, et al. (2011) A prospective natural-history study of coronary atherosclerosis. N Engl J Med 364: 226-235.

90) Cao M, Khan J A, Kang B Y, Mehta J L, Hermonat P L. (2012) Dual AAV/IL-10 plus STAT3 anti-inflammatory gene delivery lowers atherosclerosis in LDLR KO mice, but without increased benefit. Int J Vasc Med. 2012: 524235.

91) Hermonat P L, Quirk J G, Bishop B M, Han L. (1997) Packaging capacity of adeno-associated virus and the potential for wild type-plus AAV gene therapy vectors. FEBS Letters 407:78-84.

92) House S L, Bolte C, Zhou M, Doetschman T, Klevitsky R, Newman G, Schultz Jel J. Cardiac-specific overexpression of fibroblast growth factor-2 protects against myocardial dysfunction and infarction in a murine model of low-flow ischemia. Circulation. 2003; 108:3140-8.

93) Chen H, Li D, Saldeen T, Mehta J L. TGF-beta 1 attenuates myocardial ischemia-reperfusion injury via inhibition of upregulation of MMP-1. Am J Physiol Heart Circ Physiol. 2003; 284:H1612-7.

94) Yang B C, Zander D S, Mehta J L. Hypoxia-reoxygenation-induced apoptosis in cultured adult rat myocytes and the protective effect of platelets and transforming growth factor-beta(1). J Pharmacol Exp Ther. 1999; 291:733-8.

95) Gamble J R, Khew-Goodall Y, Vadas M A. Transforming growth factor-beta inhibits E-selectin expression on human endothelial cells. J Immunol. 1993; 150: 4494-503.

96) Argmann C A, Van Den Diepstraten C H, Sawyez C G, Edwards J Y, Hegele R A, Wolfe B M, Huff M W. Transforming growth factor-betal inhibits macrophage cholesteryl ester accumulation induced by native and oxidized VLDL remnants. Arterioscler Thromb Vasc Biol. 2001; 21:2011 8.

97) Chen H, Li D, Saldeen T, Mehta J L. Transforming growth factor-beta(1) modulates oxidatively modified LDL-induced expression of adhesion molecules: role of LOX-1. Circ Res. 2001; 89:1155-60.

98) Mallat Z, Tedgui A. The role of transforming growth factor beta in atherosclerosis: novel insights and future perspectives. Curr Opin Lipidol. 2002; 13: 523-9.

99) Goj ova A, Brun V, Esposito B, Cottrez F, Gourdy P, Ardouin P, Tedgui A, Mallat Z, Groux H. Specific abrogation of transforming growth factor-{beta} signaling in T cells alters atherosclerotic lesion size and composition in mice. Blood. 2003.

100) Prud'homme G. Pathobiology of transforming growth factor β in cancer, fibrosis and immunologic disease, and therapeutic considerations. Lab Invest 87: 1077-1091, 2007.

101) Li D, Liu Y, Chen J, Velchala N, Amani F, Nemarkommula A, Chen K, Rayaz H, Zhang D, Liu H, Sinha A K, Romeo F, Hermonat P L, Mehta J L. Suppression of atherogenesis by delivery of TGFbetal ACT using adeno-associated virus type 2 in LDLR knockout mice. Biochem Biophys Res Commun. 2006; 344(3):701-7.

102) Wahl, S M, Swisher, J.,and Chen W. (2004) TGFbeta: the perpetrator of immune suppression by regulatory T cells and suicidal T cells. J. Leukoc. Biology 76:15-24.

103) Yang, X., Letterio, J. J., Lechleider, R. J., Chen, L., Hayman, R., Gu, H., Roberts, A. B., Deng, C. (1999) Targeted disruption of SMAD3 results in impaired mucosal immunity and diminished T cell responsiveness to TGF-β EMBO 1 18,1280-1291.

104) Meng X-M, Huang H R, Xiao J, Chung A C K, Qin W, Chen H, Lan H Y. Disruption of Smad4 impairs TGF-β/Smad3 and Smad7 transcriptional regulation during renal inflammation and fibrosis in vivo and in vitro. Kidney Internat. 2012, 81: 266-279.˜10˜

105) Zawel L, Dai J L, Buckhaults P, Zhou S, Kinzler K W, Vogelstein B, Kern S E. Human Smad3 and Smad4 are sequence-specific transcription activators. Mol Cell 1998, 1:611-617.

106) Guo B. Slevin M. Li C. Parameshwar S. Liu D. Kumar P. Bernabeu C. Kumar S. CD105 inhibits transforming growth factor-beta-Smad3 signalling. Anticancer Research. 24(3a):1337-45, 2004.

107) Ryer E J. Hom R P. Sakakibara K. Nakayama K I. Nakayama K. Faries P L. Liu B. Kent K C. PKCdelta is necessary for Smad3 expression and transforming growth factor beta-induced fibronectin synthesis in vascular smooth muscle cells. Arteriosclerosis, Thrombosis & Vascular Biology. 26(4):780-6, 2006.

108) You C X, Liu Y, Shi M, Cao M, Luo R-C, Hermonat P L. Comparison of AAV/IL-7 autocrine (T cell) versus paracrine (DC) gene delivery for enhancing CTL stimulation and function. (2010) In press Cancer Imm & Immunother.

109) Kislauskis E H, Zhu X, Singer R H Beta-actin messenger RNA localization and protein synthesis augment cell motility. J Cell Biology1997, 136: 1263-1270.

110) Maeda H, Shiraishi A. TGF-beta contributes to the shift toward Th2-type responses through direct and IL-10-mediated pathways in tumor bearing mice. J Immunol 156: 73-78, 1996.

111) Chyu K Y, Nilsson J, Shah P K. Immune mechanisms in atherogenesis and potential for an atherosclerosis vaccine. Discov Med 11: 403-412, 2011.

112) Tsuchida, Nakatani M, Hitachi K, Uezumi A, Sunada Y, Ageta H, Inokuchi K. Activin signaling as an emerging target for therapeutic interventions. Cell Comm Signal 7:15, 2009.

113) Ruiz-Ortega M, Ruperez M, Esteban V, Rodriguez-Vita J, Sanchez-Lopez E, Carvajal G, Egido J. Angiotensin II: a key factor in inflammatory and fibrotic response in kidney diseases. Neph Dialysis Transplant 21: 16-20, 2006.

114) Masszi A, Kapu A. Smaddening complexity: the role of Smad3 in epithelial-myofibroblast transition. Cells Tiss Organs 193:41-52, 2011.

115) Bowen G P, Borgland S L, Lam M, Libermann T A, Wong N C, Muruve D A. Adenovirus-induced inflammation: capsid-dependent induction of the C-C chemokine RANTES requires NF-kappa B. Hum Gene Ther. 2002 Feb. 10; 13(3):367-79.

116) Leivonen S-K, Chantry A, Hakkinen L, Han J, Kahari V-M. Smad3 mediates transforming growth factor-β-induced collagenase-3 (Matrix Metalloproteinase-13) expression in human gingival fibroblasts. J Biolog Chem 277: 46338-46346. 2002.˜11˜

117) Kim E-J, Cho H-J, Park D, Kim J Y, Kim Y B, Park T G, Shim C-K, Oh Y-K. Antifibrotic effect of MMP13-encoding plasmid DNA delivered using polyethylenimine shielded with hyaluronic acid. Molec Ther 12: 355-362, 2011.

118) Kundi, R, Hollenbeck S T, Yamanouchi D, Herman B C, Edlin R, Ryer E J, Wang C, Tsai S, Liu S, Kent K C. Arterial gene transfer of the TGF-beta signaling protein Smad3 induces adaptive remodeling following angioplasty: a role for CTGF. Cardiovascular Research 2009, 84: 326-335.

119) Liu Q, Muruve D A. Molecular basis of the inflammatory response to adenovirus vectors. Gene therapy 2003, 10:935-940.

120) Kawai T, Fujiwara T, Aoyama Y, Aizawa Y, Yamada Y, Aoyagi T, Mikata A, Kageyama K. Diffuse interstitial fibrosing pneumonitis and adenovirus infection. Chest 1976; 69;692-694.

121) Keddis M, Leung N, Herrmann S, El-Zoghby Z, Sethi S. Adenovirus-induced interstitial nephritis following umbilical cord blood transplant for chronic lymphocytic leukemia. Am J Kid Dis. 59:886-890, 2012.

122) NIH Report. Assessment of adenoviral vector safety and toxicity: Report of the National Institutes of Health Recombinant DNA Advisory Committee. Hum Gene Ther 2002, 13: 3-13.

123) Libby P (2002) Inflammation in atherosclerosis. Nature 420(6917):868-874.

124) Hou X, Song J, Su J, Huang D, Gao W, Yan J et al (2015) CD4(+)Foxp3(+) Tregs protect against innate immune cell-mediated fulminant hepatitis in mice. Mol Immunol 63(2):420-427.

125) Mahajan D, Wang Y, Qin X, Wang Y, Zheng G, Wang YM et al (2006) CD4+CD25+regulatory T cells protect against injury in an innate murine model of chronic kidney disease. J Am Soc Nephrol 17(10):2731-2741.

126) Bacchetta R, Passerini L, Gambineri E, Dai M, Allan S E, Perroni L et al (2006) Defective regulatory and effector T cell functions in patients with FOXP3 mutations. J Clin Invest 116:1713-1722.

127) Roncarolo M G Battaglia M (2007) Regulatory T-cell immunotherapy for tolerance to self antigens and alloantigens in humans. Nat Rev Immunol 7:585-598.

128) Quintana F J, Iglesias A H, Farez M F, Caccamo M, Burns E J, Kassam N et al (2010) Adaptive autoimmunity and Foxp3-based immunoregulation in zebrafish. PLoS One 5(3):e9478.

129) Hermonat P L (2014) Adeno-associated virus-based transgene delivery for treating progressive vascular diseases. Clon Transgen 3:e111.

130) Hon S, Nomura T, Sakaguchi S (2003) Control of regulatory T cell development by the transcription factor Foxp3. Science 299:1057-1061.

131) Han X, Kitamoto S, Wang H, Boisvert W A (2010) Interleukin-10 overexpression in macrophages suppresses atherosclerosis in hyperlipidemic mice. FASEB J 24:2869-2880.

132) Chung H Y, Kim H J, Kim J W, Yu B P (2006) The inflammation hypothesis of aging. Annals NY Acad Sci 928:327-336.

133) Kregel K C, Zhang H J (2007) An integrated view of oxidative stress in aging: basic mechanisms, functional effects, and pathological considerations. Am J Physiol Regul Comp Physiol 292:R18-R36.

134) Chung H Y, Cesari M, Anton S, Marzetti E, Giovannini S, Seo A Y et al (2009) Leeuwenburgh. Molecular inflammation: Underpinnings of aging and age-related diseases. Aging Res Rev 8:18-30.

135) Klingenberg R, Gerdes N, Badeau R M, Gistera A, Strothoff D, Ketelhuth D F et al (2013) Depletion of Foxp3 +regulatory T cells promotes hypercholesterolemia and atherosclerosis. J Clin Investig 123:1323-1334.

SEQUENCE LISTING

```
LOX1 promoter: 2.4 kb (derived from GenBank Accession No. AH00781.1)
SEQ ID NO: 1
atgaggccca cctacattat gcagcgaaat ctactttcct ctgctgatta atatgttaat
ctcatttaaa agcatcctgt ctgggaaccc catgacccag tcaagaagac acagaaggcc
agcacggtgg ctcacggctt gtaatcccag cactgtggga ggctgaggca ggcggatcac
ttgaggacag gagttcaaga ccagcctggc caacatggtg aaaccctgtc tctactgaaa
atacaaaaca attagccggg aggggtggcg ggcacctgta atcccagcta ctcaggaggc
tgaggcagga gaattgcttg aacccaggag gcggaggttg cagtgagccg agatcatgcc
attgcaaccc accctgggta acaagggcga aactccatct caaaaacaaa caaacaaaca
aacaaacaaa caaaacacat gaaattcacc atcacaggag ttattgcttg gtgggtatag
agtttcagtt ggggaagatg aaaagttctg gagatggatg gtggtgatgg ctgcacaaca
atgtaaatgt acttaatgcc actgaacagt acgcctaaaa atggttgaaa tggtaaacac
tttatgttct ataatattca tggcatccaa aataactgag tcactgtaga aatgataatg
tgcccttttc agtgtgatat cgtttcagta ttttttaatt cttttttttt ttttttttaa
agggagtctc gctctgttgc ccaggctgga gtacagtggc acaatctcag ctcactgaaa
atgaaaagca ttttcatttt ccacctccca aatagctggg attataggct tgcaaccagg
cccagctaac ttttgtattt ttagtggtga cagggtttca acaggttggc caggctggtc
tcgaactcct gacctcagtg atccaccccc ccctcagctt cccaaagtgc tgagattaca
ggcatgagcc actgcacctg gccaactttt tcaatttgtt ctcaaatact ctgcacataa
taaaaggaat ataggaaaca gggaggagga gggaaatgga atcctaatta ggctgtcaat
attggtaatt tttcctaata gtttcctata tgttttattt atgtatttat ttttccattc
ttcctttgtg tattcttttc tttgcacttt tttttcctgg agtaatttaa agtaaatgcc
agatatcata tcattttgct tataaatact tccttcagaa gacatctcta atagatatat
atctggttag gaggttgtca agggaaaata gaaaagtatt ctatcagtaa atacatatag
acatacttat gggtctctca tgtaagcgtc tttgtgacat gagtcaatca tggattttac
tgaaatacat tatttgatgg gtaattcaac tgtgctgaat attagtacta tactaataat
tattatttat tggcttctta ttatgctcca agtacaaagc ttgtcatttt taaccagcgt
gtcatttgaa ttcctatagc aacgtgacat ttttcacaaa cctagtgtaa aaaagcttat
tatttttacc cagcatgtca tttgaattcc tataacaatg ttatcaggta gacattatcc
ctaatttcca gacatgtcat ttgaattcct ataacaatgt tatcaggtag acattatccc
taatttccag acaaagacat agattcaaag tattaagatt tcccaagatt gtagactaaa
ggaaagtact gaaactagct ctttctgcat tcaaagtgct ctctgctgct acgttacact
ggcactgcat gaactagtag ccccagataa attctaaaat cacccaggac acaccatcgc
tggaaatggg agccatatgt gattcacccc aaaattctta gatccattgc cagataaaac
agtgataatc aaaagagaat atatctgatt cataaaagac accatataga aacagtctgc
taatgaaatt agcaaacaaa cattggaatc aaatattttg gcccataatt ctggcacatt
ttttacaaat gtagtgtgac ttactctctt tgaatttcag tttctgtctc tgaagagtgg
gtacaatatc tcctctgatg ctcatgaaaa atagttttcc ctttcataaa ttacttagcg
aaatatcctg aaacaccttc agaatcacca ctttctccac ctgcaataca cataactcaa
gaatttgcgt cagcgaactt cccaatatga agcaaagcct ctccttcctc ctaccaatga
ttgagccatt cttctattag ataacagtag ctatttaaat acttctgcag aagctcacat
atttttagtt tgttgaagtt cgtgactgct tcactctctc attcttagct tgaatttgga
aatgactttt gatgacctaa
Human IL10 coding (nucleotide) sequence (derived from NCBI Reference
Sequence (RefSeq) NM_000572)
SEQ ID NO: 2
aaagaaggca tgcacagctc agcactgctc tgttgcctgg tcctcctgac tggggtgagg
gccagcccag gccagggcac ccagtctgag aacagctgca cccacttccc aggcaacctg
cctaacatgc ttcgagatct ccgagatgcc ttcagcagag tgaagacttt ctttcaaatg
aaggatcagc tggacaactt gttgttaaag gagtccttgc tggaggactt taagggttac
ctgggttgcc aagccttgtc tgagatgatc cagttttacc tggaggaggt gatgccccaa
gctgagaacc aagacccaga catcaaggcg catgtgaact ccctggggga gaacctgaag
accctcaggc tgaggctacg gcgctgtcat cgatttcttc cctgtgaaaa caagagcaag
gccgtggagc aggtgaagaa tgcctttaat aagctccaag
SEQ ID NOs: 3 and 4
SEQ ID NO: 3:
AAV8 capsid coding (nucleotide) sequence with Tyr447 and Tyr 733
mutated to Phe residues (bolded and in capital letters)
SEQ ID NO: 4:
AAV8 capsid amino acid sequence with Tyr447 and Tyr 733 mutated to
Phe residues (bolded and in capital letters)
atggctgccgatggttatcttccagattggctcgaggacaacctc
  M A A D G Y L P D W L E D N L
tctgagggcattcgcgagtggtgggcgctgaaacctggagccccg
  S E G I R E W W A L K P G A P
aagcccaaagccaaccagcaaaagcaggacgacggccggggtctg
  K P K A N Q Q K Q D D G R G L
gtgcttcctggctacaagtacctcggacccttcaacggactcgac
  V L P G Y K Y L G P F N G L D
aagggggagcccgtcaacgcggcggacgcagcggccctcgagcac
  K G E P V N A A D A A A L E H
gacaaggcctacgaccagcagctgcaggcgggtgacaatccgtac
  D K A Y D Q Q L Q A G D N P Y
ctgcggtataaccacgccgacgccgagtttcaggagcgtctgcaa
  L R Y N H A D A E F Q E R L Q
gaagatacgtcttttgggggcaacctcgggcgagcagtcttccag
  E D T S F G G N L G R A V F Q
gccaagaagcgggttctcgaacctctcggtctggttgaggaaggc
  A K K R V L E P L G L V E E G
gctaagacggctcctggaaagaagagaccggtagagccatcaccc
  A K T A P G K K R P V E P S P
cagcgttctccagactcctctacgggcatcggcaagaaaggccaa
  Q R S P D S S T G I G K K G Q
cagcccgccagaaaaagactcaattttggtcagactggcgactca
  Q P A R K R L N F G Q T G D S
gagtcagttccagaccctcaacctctcggagaacctccagcagcg
  E S V P D P Q P L G E P P A A
ccctctggtgtgggacctaatacaatggctgcaggcggtggcgca
  P S G V G P N T M A A G G G A
ccaatggcagacaataacgaaggcgccgacggagtgggtagttcc
  P M A D N N E G A D G V G S S
tcgggaaattggcattgcgattccacatggctgggcgacagagtc
  S G N W H C D S T W L G D R V
atcaccaccagcacccgaacctgggccctgcccacctacaacaac
  I T T S T R T W A L P T Y N N
cacctctacaagcaaatctccaacgggacatcgggaggagccacc
  H L Y K Q I S N G T S G G A T
aacgacaacacctacttcggctacagcaccccctgggggtatttt
  N D N T Y F G Y S T P W G Y F
gactttaacagattccactgccacttttcaccacgtgactggcag
  D F N R F H C H F S P R D W Q
cgactcatcaacaacaactggggattccggcccaagagactcagc
  R L I N N N W G F R P K R L S
ttcaagctcttcaacatccaggtcaaggaggtcacgcagaatgaa
  F K L F N I Q V K E V T Q N E
ggcaccaagaccatcgccaataacctcaccagcaccatccaggtg
  G T K T I A N N L T S T I Q V
tttacggactcggagtaccagctgccgtacgttctcggctctgcc
  F T D S E Y Q L P Y V L G S A
caccagggctgcctgcctccgttcccggcggacgtgttcatgatt
  H Q G C L P P F P A D V F M I
ccccagtacggctacctaacactcaacaacggtagtcaggccgtg
  P Q Y G Y L T L N N G S Q A V
ggacgctcctccttctactgcctggaatactttccttcgcagatg
  G R S S F Y C L E Y F P S Q M
ctgagaaccggcaacaacttccagtttacttacaccttcgaggac
  L R T G N N F Q F T Y T F E D
gtgcctttccacagcagctacgcccacagccagagcttggaccgg
  V P F H S S Y A H S Q S L D R
ctgatgaatcctctgattgaccagtacctgtacTTCttgtctcgg
  L M N P L I D Q Y L Y F L S R
actcaaacaacaggaggcacggcaaatacgcagactctgggcttc
  T Q T T G G T A N T Q T L G F
agccaaggtgggcctaatacaatggccaatcaggcaaagaactgg
  S Q G G P N T M A N Q A K N W
ctgccaggaccctgttaccgccaacaacgcgtctcaacgacaacc
  L P G P C Y R Q Q R V S T T T
gggcaaaacaacaatagcaactttgcctggactgctgggaccaaa
  G Q N N N S N F A W T A G T K
taccatctgaatggaagaaattcattggctaatcctggcatcgct
  Y H L N G R N S L A N P G I A
atggcaacacacaaagacgacgaggagcgtttttttcccagtaac
  M A T H K D D E E R F F P S N
gggatcctgatttttggcaaacaaaatgctgccagagacaatgcg
  G I L I F G K Q N A A R D N A
gattacagcgatgtcatgctcaccagcgaggaagaaatcaaaacc
  D Y S D V M L T S E E E I K T
actaaccctgtggctacagaggaatacggtatcgtggcagataac
  T N P V A T E E Y G I V A D N
ttgcagcagcaaaacacggctcctcaaattggaactgtcaacagc
  L Q Q Q N T A P Q I G T V N S
cagggggccttacccggtatggtctggcagaaccgggacgtgtac
  Q G A L P G M V W Q N R D V Y
ctgcagggtcccatctgggccaagattcctcacacggacggcaac
  L Q G P I W A K I P H T D G N
ttccacccgtctccgctgatgggcggctttggcctgaaacatcct
  F H P S P L M G G F G L K H P
ccgcctcagatcctgatcaagaacacgcctgtacctgcggatcct
  P P Q I L I K N T P V P A D P
ccgaccaccttcaaccagtcaaagctgaactctttcatcacgcaa
  P T T F N Q S K L N S F I T Q
tacagcaccggacaggtcagcgtggaaattgaatgggagctgcag
  Y S T G Q V S V E I E W E L Q
aaggaaaacagcaagcgctggaaccccgagatccagtacacctcc
  K E N S K R W N P E I Q Y T S
aactactacaaatctacaagtgtggactttgctgttaatacagaa
  N Y Y K S T S V D F A V N T E
ggcgtgtactctgaaccccgccccattggcacccgtTTCctcacc
  G V Y S E P R P I G T R F L T
cgtaatctgtaa 2217
  R N L *
SEQ ID NOs: 5 and 6
SEQ ID NO: 5:
AAV8 capsid coding (nucleotide) sequence with EYH modification
(bolded and in capital letters) showing 7 additional amino acids
(EYHHYNK) being inserted after position 590, as well as adjacent changes
SEQ ID NO: 6:
AAV8 capsid amino acid sequence with EYH modification (bolded and
in capital letters) showing 7 additional amino acids (EYHHYNK) being
inserted after position 590, as well as adjacent changes
atggctgccgatggttatcttccagattggctcgaggacaacctc
  M A A D G Y L P D W L E D N L
tctgagggcattcgcgagtggtgggcgctgaaacctggagccccg
  S E G I R E W W A L K P G A P
aagcccaaagccaaccagcaaaagcaggacgacggccggggtctg
  K P K A N Q Q K Q D D G R G L
gtgcttcctggctacaagtacctcggacccttcaacggactcgac
  V L P G Y K Y L G P F N G L D
aagggggagcccgtcaacgcggcggacgcagcggccctcgagcac
  K G E P V N A A D A A A L E H
gacaaggcctacgaccagcagctgcaggcgggtgacaatccgtac
  D K A Y D Q Q L Q A G D N P Y
ctgcggtataaccacgccgacgccgagtttcaggagcgtctgcaa
  L R Y N H A D A E F Q E R L Q
gaagatacgtcttttgggggcaacctcgggcgagcagtcttccag
  E D T S F G G N L G R A V F Q
gccaagaagcgggttctcgaacctctcggtctggttgaggaaggc
  A K K R V L E P L G L V E E G
gctaagacggctcctggaaagaagagaccggtagagccatcaccc
  A K T A P G K K R P V E P S P
cagcgttctccagactcctctacgggcatcggcaagaaaggccaa
  Q R S P D S S T G I G K K G Q
cagcccgccagaaaaagactcaattttggtcagactggcgactca
  Q P A R K R L N F G Q T G D S
gagtcagttccagaccctcaacctctcggagaacctccagcagcg
  E S V P D P Q P L G E P P A A
ccctctggtgtgggacctaatacaatggctgcaggcggtggcgca
  P S G V G P N T M A A G G G A
ccaatggcagacaataacgaaggcgccgacggagtgggtagttcc
  P M A D N N E G A D G V G S S
tcgggaaattggcattgcgattccacatggctgggcgacagagtc
  S G N W H C D S T W L G D R V
atcaccaccagcacccgaacctgggccctgcccacctacaacaac
  I T T S T R T W A L P T Y N N
cacctctacaagcaaatctccaacgggacatcgggaggagccacc
  H L Y K Q I S N G T S G G A T
aacgacaacacctacttcggctacagcaccccctgggggtatttt
  N D N T Y F G Y S T P W G Y F
gactttaacagattccactgccacttttcaccacgtgactggcag
  D F N R F H C H F S P R D W Q
cgactcatcaacaacaactggggattccggcccaagagactcagc
  R L I N N N W G F R P K R L S
ttcaagctcttcaacatccaggtcaaggaggtcacgcagaatgaa
  F K L F N I Q V K E V T Q N E
ggcaccaagaccatcgccaataacctcaccagcaccatccaggtg
  G T K T I A N N L T S T I Q V
tttacggactcggagtaccagctgccgtacgttctcggctctgcc
  F T D S E Y Q L P Y V L G S A
caccagggctgcctgcctccgttcccggcggacgtgttcatgatt
  H Q G C L P P F P A D V F M I
ccccagtacggctacctaacactcaacaacggtagtcaggccgtg
  P Q Y G Y L T L N N G S Q A V
ggacgctcctccttctactgcctggaatactttccttcgcagatg
  G R S S F Y C L E Y F P S Q M
ctgagaaccggcaacaacttccagtttacttacaccttcgaggac
  L R T G N N F Q F T Y T F E D
gtgcctttccacagcagctacgcccacagccagagcttggaccgg
  V P F H S S Y A H S Q S L D R
ctgatgaatcctctgattgaccagtacctgtactacttgtctcgg
  L M N P L I D Q Y L Y Y L S R
actcaaacaacaggaggcacggcaaatacgcagactctgggcttc
  T Q T T G G T A N T Q T L G F
agccaaggtgggcctaatacaatggccaatcaggcaaagaactgg
  S Q G G P N T M A N Q A K N W
ctgccaggaccctgttaccgccaacaacgcgtctcaacgacaacc
  L P G P C Y R Q Q R V S T T T
gggcaaaacaacaatagcaactttgcctggactgctgggaccaaa
  G Q N N N S N F A W T A G T K
taccatctgaatggaagaaattcattggctaatcctggcatcgct
  Y H L N G R N S L A N P G I A
atggcaacacacaaagacgacgaggagcgtttttttcccagtaac
  M A T H K D D E E R F F P S N
gggatcctgatttttggcaaacaaaatgctgccagagacaatgcg
  G I L I F G K Q N A A R D N A
gattacagcgatgtcatgctcaccagcgaggaagaaatcaaaacc
  D Y S D V M L T S E E E I K T
actaaccctgtggctacagaggaatacggtatcgtggcagataac
  T N P V A T E E Y G I V A D N
ttgcagGGAcaaCGA
  L Q G Q R
gaataccaccactacaacaaa
  E Y H H Y N K (this is the EYH insertion with
                                                                                            adjacent changes)
gcacaagcagca caaattggaactgtcaacagc
  A Q A A Q I G T V N S
1801 cagggggccttacccggtatggtctggcagaaccgggacgtgtac
            Q G A L P G M V W Q N R D V Y
          ctgcagggtcccatctgggccaagattcctcacacggacggcaac
            L Q G P I W A K I P H T D G N
          ttccacccgtctccgctgatgggcggctttggcctgaaacatcct
            F H P S P L M G G F G L K H P
          ccgcctcagatcctgatcaagaacacgcctgtacctgcggatcct
            P P Q I L I K N T P V P A D P
          ccgaccaccttcaaccagtcaaagctgaactctttcatcacgcaa
            P T T F N Q S K L N S F I T Q
          tacagcaccggacaggtcagcgtggaaattgaatgggagctgcag
            Y S T G Q V S V E I E W E L Q
          aaggaaaacagcaagcgctggaaccccgagatccagtacacctcc
            K E N S K R W N P E I Q Y T S
          aactactacaaatctacaagtgtggactttgctgttaatacagaa
            N Y Y K S T S V D F A V N T E
          ggcgtgtactctgaaccccgccccattggcacccgttacctcacc
            G V Y S E P R P I G T R Y L T
          cgtaatctgtaa 2217
            R N L *
SEQ ID NOs: 7 and 8
SEQ ID NO: 7:
AAV8 capsid coding (nucleotide) sequence dual modifications: (1)
Tyr447 and Tyr 733 mutated to Phe residues (bolded and in capital
letters) (2) with EYH modification (bolded and in capital letters)
showing 7 additional amino acids (EYHHYNK) being inserted after
position 590, as well as adjacent changes
SEQ ID NO: 8:
AAV8 capsid amino acid sequence dual modifications: (1) Tyr447 and
Tyr 733 mutated to Phe residues (bolded and in capital letters)
(2) with EYH modification (bolded and in capital letters) showing
7 additional amino acids (EYHHYNK) being inserted after position
590, as well as adjacent changes
atggctgccgatggttatcttccagattggctcgaggacaacctc
  M A A D G Y L P D W L E D N L
tctgagggcattcgcgagtggtgggcgctgaaacctggagccccg
  S E G I R E W W A L K P G A P
aagcccaaagccaaccagcaaaagcaggacgacggccggggtctg
  K P K A N Q Q K Q D D G R G L
gtgcttcctggctacaagtacctcggacccttcaacggactcgac
  V L P G Y K Y L G P F N G L D
aagggggagcccgtcaacgcggcggacgcagcggccctcgagcac
  K G E P V N A A D A A A L E H
gacaaggcctacgaccagcagctgcaggcgggtgacaatccgtac
  D K A Y D Q Q L Q A G D N P Y
ctgcggtataaccacgccgacgccgagtttcaggagcgtctgcaa
  L R Y N H A D A E F Q E R L Q
gaagatacgtcttttgggggcaacctcgggcgagcagtcttccag
  E D T S F G G N L G R A V F Q
gccaagaagcgggttctcgaacctctcggtctggttgaggaaggc
  A K K R V L E P L G L V E E G
gctaagacggctcctggaaagaagagaccggtagagccatcaccc
  A K T A P G K K R P V E P S P
cagcgttctccagactcctctacgggcatcggcaagaaaggccaa
  Q R S P D S S T G I G K K G Q
cagcccgccagaaaaagactcaattttggtcagactggcgactca
  Q P A R K R L N F G Q T G D S
gagtcagttccagaccctcaacctctcggagaacctccagcagcg
  E S V P D P Q P L G E P P A A
ccctctggtgtgggacctaatacaatggctgcaggcggtggcgca
  P S G V G P N T M A A G G G A
ccaatggcagacaataacgaaggcgccgacggagtgggtagttcc
  P M A D N N E G A D G V G S S
tcgggaaattggcattgcgattccacatggctgggcgacagagtc
  S G N W H C D S T W L G D R V
atcaccaccagcacccgaacctgggccctgcccacctacaacaac
  I T T S T R T W A L P T Y N N
cacctctacaagcaaatctccaacgggacatcgggaggagccacc
  H L Y K Q I S N G T S G G A T
aacgacaacacctacttcggctacagcaccccctgggggtatttt
  N D N T Y F G Y S T P W G Y F
gactttaacagattccactgccacttttcaccacgtgactggcag
  D F N R F H C H F S P R D W Q
cgactcatcaacaacaactggggattccggcccaagagactcagc
  R L I N N N W G F R P K R L S
ttcaagctcttcaacatccaggtcaaggaggtcacgcagaatgaa
  F K L F N I Q V K E V T Q N E
ggcaccaagaccatcgccaataacctcaccagcaccatccaggtg
  G T K T I A N N L T S T I Q V
tttacggactcggagtaccagctgccgtacgttctcggctctgcc
  F T D S E Y Q L P Y V L G S A
caccagggctgcctgcctccgttcccggcggacgtgttcatgatt
  H Q G C L P P F P A D V F M I
ccccagtacggctacctaacactcaacaacggtagtcaggccgtg
  P Q Y G Y L T L N N G S Q A V
ggacgctcctccttctactgcctggaatactttccttcgcagatg
  G R S S F Y C L E Y F P S Q M
ctgagaaccggcaacaacttccagtttacttacaccttcgaggac
  L R T G N N F Q F T Y T F E D
gtgcctttccacagcagctacgcccacagccagagcttggaccgg
  V P F H S S Y A H S Q S L D R
ctgatgaatcctctgattgaccagtacctgtacTTCttgtctcgg
  L M N P L I D Q Y L Y F L S R
actcaaacaacaggaggcacggcaaatacgcagactctgggcttc
  T Q T T G G T A N T Q T L G F
agccaaggtgggcctaatacaatggccaatcaggcaaagaactgg
  S Q G G P N T M A N Q A K N W
ctgccaggaccctgttaccgccaacaacgcgtctcaacgacaacc
  L P G P C Y R Q Q R V S T T T
gggcaaaacaacaatagcaactttgcctggactgctgggaccaaa
  G Q N N N S N F A W T A G T K
taccatctgaatggaagaaattcattggctaatcctggcatcgct
  Y H L N G R N S L A N P G I A
atggcaacacacaaagacgacgaggagcgtttttttcccagtaac
  M A T H K D D E E R F F P S N
gggatcctgatttttggcaaacaaaatgctgccagagacaatgcg
  G I L I F G K Q N A A R D N A
gattacagcgatgtcatgctcaccagcgaggaagaaatcaaaacc
  D Y S D V M L T S E E E I K T
actaaccctgtggctacagaggaatacggtatcgtggcagataac
  T N P V A T E E Y G I V A D N
ttgcagGGAcaaCGA
  L Q G Q R
GAATACCACCACTACAACAAA
  E Y H H Y N K (this is the EYH insertion with
                                                      adjacent changes)
GCACAAGCAGCA caaattggaactgtcaacagc
  A Q A A Q I G T V N S
cagggggccttacccggtatggtctggcagaaccgggacgtgtac
  Q G A L P G M V W Q N R D V Y
ctgcagggtcccatctgggccaagattcctcacacggacggcaac
  L Q G P I W A K I P H T D G N
ttccacccgtctccgctgatgggcggctttggcctgaaacatcct
  F H P S P L M G G F G L K H P
ccgcctcagatcctgatcaagaacacgcctgtacctgcggatcct
  P P Q I L I K N T P V P A D P
ccgaccaccttcaaccagtcaaagctgaactctttcatcacgcaa
  P T T F N Q S K L N S F I T Q
tacagcaccggacaggtcagcgtggaaattgaatgggagctgcag
  Y S T G Q V S V E I E W E L Q
aaggaaaacagcaagcgctggaaccccgagatccagtacacctcc
  K E N S K R W N P E I Q Y T S
aactactacaaatctacaagtgtggactttgctgttaatacagaa
  N Y Y K S T S V D F A V N T E
ggcgtgtactctgaaccccgccccattggcacccgtTTCctcacc
  G V Y S E P R P I G T R F L T
cgtaatctgtaa 2217
  R N L *
Remains of pGL3 polylinker:
SEQ ID NO: 9
ggtaccgagc tcttacgcgt gctagccc ctgcag
(KpnI NheI PstI)
LT (left-terminal) ITR (Inverted Terminal Repeat):
SEQ ID NO: 10
ttggccactc cctctctgcg cgctcgctcg ctcactgagg ccgggcgacc aaaggtcgcc
cgacgcccgg gctttgcccg ggcggcctca gtgagcgagc gagcgcgcag agagggagtg
gccaactcca tcactagggg ttcct
Sequence between ITRs:
SEQ ID NO: 11
gcggccgc atgcat agatct acgcgt ctcgag
(NotI NsiI BglII MluI XhoI)
RT (right-terminal) ITR (Inverted Terminal Repeat) plus remains of pGL3:
SEQ ID NO: 12
aggaac ccctagtgat ggagttggcc actccctctc tgcgcgctcg ctcgctcact
gaggccgggc gaccaaaggt cgcccgacgc ccgggctttg cccgggcggc ctcagtgagc
gagcgagcgc gcagagaggg agtggccaa ctgcag gaattc ggatccg tcgaccgatg
cccttgagag ccttcaaccc
(PstI EcoRI BamHI-remains of pGL3)
agtcagctcc ttccggtggg cgcggggcat gactatcgtc gccgcactta tgactgtctt
ctttatcatg caactcgtag gacaggtgcc ggcagcgctc ttccgcttcc tcgctcactg
actcgctgcg ctcggtcgtt cggctgcggc gagcggtatc agctcactca aaggcggtaa
tacggttatc cacagaatca ggggataacg caggaaagaa catgtgagca aaaggccagc
aaaaggccag gaaccgtaaa aaggccgcgt tgctggcgtt tttccatagg ctccgccccc
ctgacgagca tcacaaaaat cgacgctcaa gtcagaggtg gcgaaacccg acaggactat
aaagatacca ggcgtttccc cctggaagct ccctcgtgcg ctctcctgtt ccgaccctgc
cgcttaccgg atacctgtcc gcctttctcc cttcgggaag cgtggcgctt tctcaatgct
cacgctgtag gtatctcagt tcggtgtagg tcgttcgctc caagctgggc tgtgtgcacg
aaccccccgt tcagcccgac cgctgcgcct tatccggtaa ctatcgtctt gagtccaacc
cggtaagaca cgacttatcg ccactggcag cagccactgg taacaggatt agcagagcga
ggtatgtagg cggtgctaca gagttcttga agtggtggcc taactacggc tacactagaa
ggacagtatt tggtatctgc gctctgctga agccagttac cttcggaaaa agagttggta
gctcttgatc cggcaaacaa accaccgctg gtagcggtgg tttttttgtt tgcaagcagc
agattacgcg cagaaaaaaa ggatctcaag aagatccttt gatcttttct acggggtctg
acgctcagtg gaacgaaaac tcacgttaag ggattttggt catgagatta tcaaaaagga
tcttcaccta gatcctttta aattaaaaat gaagttttaa atcaatctaa agtatatatg
agtaaacttg gtctgacagt taccaatgct taatcagtga ggcacctatc tcagcgatct
gtctatttcg ttcatccata gttgcctgac tccccgtcgt gtagataact acgatacggg
agggcttacc atctggcccc agtgctgcaa tgataccgcg agacccacgc tcaccggctc
cagatttatc agcaataaac cagccagccg gaagggccga gcgcagaagt ggtcctgcaa
ctttatccgc ctccatccag tctattaatt gttgccggga agctagagta agtagttcgc
cagttaatag tttgcgcaac gttgttgcca ttgctacagg catcgtggtg tcacgctcgt
cgtttggtat ggcttcattc agctccggtt cccaacgatc aaggcgagtt acatgatccc
ccatgttgtg caaaaaagcg gttagctcct tcggtcctcc gatcgttgtc agaagtaagt
tggccgcagt gttatcactc atggttatgg cagcactgca taattctctt actgtcatgc
catccgtaag atgcttttct gtgactggtg agtactcaac caagtcattc tgagaatagt
gtatgcggcg accgagttgc tcttgcccgg cgtcaatacg ggataatacc gcgccacata
gcagaacttt aaaagtgctc atcattggaa aacgttcttc ggggcgaaaa ctctcaagga
tcttaccgct gttgagatcc agttcgatgt aacccactcg tgcacccaac tgatcttcag
catcttttac tttcaccagc gtttctgggt gagcaaaaac aggaaggcaa aatgccgcaa
aaaagggaat aagggcgaca cggaaatgtt gaatactcat actcttcctt tttcaatatt
attgaagcat ttatcagggt tattgtctca tgagcggata catatttgaa tgtatttaga
aaaataaaca aataggggtt ccgcgcacat ttccccgaaa agtgccacct gacgcgccct
gtagcggcgc attaagcgcg gcgggtgtgg tggttacgcg cagcgtgacc gctacacttg
ccagcgccct agcgcccgct cctttcgctt tcttcccttc ctttctcgcc acgttcgccg
gctttccccg tcaagctcta aatcgggggc tccctttagg gttccgattt agtgctttac
ggcacctcga ccccaaaaaa cttgattagg gtgatggttc acgtagtggg ccatcgccct
gatagacggt ttttcgccct ttgacgttgg agtccacgtt ctttaatagt ggactcttgt
tccaaactgg aacaacactc aaccctatct cggtctattc ttttgattta taagggattt
tgccgatttc ggcctattgg ttaaaaaatg agctgattta acaaaaattt aacgcgaatt
ttaacaaaat attaacgttt acaatttccc attcgccatt caggctgcgc aactgttggg
aagggcgatc ggtgcgggcc tcttcgctat tacgccagcc caagctacca tgataagtaa
gtaatattaa ggtacgggag gtacttggag cggccgcaat aaaatatctt tattttcatt
acatctgtgt gttggttttt tgtgtgaatc gatagtacta acatacgctc tccatcaaaa
caaaacgaaa caaaacaaac tagcaaaata ggctgtcccc agtgcaagtg caggtgccag
aacatttctc tatcgata
7-amino acid peptide that can be inserted into AAV capsid:
SEQ ID NO: 13
EYHHYNK
Upstream primer to generate Lox-1 promoter:
SEQ ID NO: 14
5′-ATATGCATCTTTCTTATTTGGGGGAAG-3′
Downstream primer to generate Lox-1 promoter:
SEQ ID NO: 15
5′-ATACGCGTACTAAAAATATGTGAGCTTCTG-3′
Human FOXP3 protein (GenBank Accession No.: AAI43786):
SEQ ID NO: 16
    1 mpnprpgkps apslalgpsp gaspswraap kasdllgarg pggtfqgrdl rggahassss
  61 lnpmppsqlq lstvdahart pvlqvhples pamisltppt tatgvfslka rpglppginv
121 aslewvsrep allctfpnps aprkdstlsa vpqssyplla ngvckwpgce kvfeepedfl
181 khcqadhlld ekgraqcllq remvqsleqq lvlekeklsa mqahlagkma ltkassvass
241 dkgsccivaa gsqgpvvpaw sgpreapdsl favrrhlwgs hgnstfpefl hnmdyfkfhn
301 mrppftyatl irwaileape kqrtlneiyh wftrmfaffr nhpatwknai rhnlslhkcf
361 vrvesekgav wtvdelefrk krsqrpsrcs nptpgp
Human FOXP3 protein (GenBank Accession No.: AAI13402):
SEQ ID NO: 17:
    1 mpnprpgkps apslalgpsp gaspswraap kasdllgarg pggtfqgrdl rggahassss
  61 lnpmppsqlq 1ptlplvmva psgarlgplp hlqallqdrp hfmhqlstvd ahartpvlqv
121 hplespamis ltppttatgv fslkarpglp pginvaslew vsrepallct fpnpsaprkd
181 stlsavpqss ypllangvck wpgcekvfee pedflkhcqa dhlldekgra qcllqremvq
241 sleqqlvlek eklsamqahl agkmaltkas svassdkgsc civaagsqgp vvpawsgpre
301 apdslfavrr hlwgshgnst fpeflhnmdy fkfhnmrppf tyatlirwai leapekqrtl
361 neiyhwftrm faffrnhpat wknairhnls lhkcfvrves ekgavwtvde lefrkkrsqr
421 psrcsnptpg p
Human FOXP3 cDNA (GenBank Accession No.: BC143785):
SEQ ID NO: 18:
      1 tcaccaagcc tgcccttgga caaggacccg atgcccaacc ccaggcctgg caagccctcg
    61 gccccttcct tggcccttgg cccatcccca ggagcctcgc ccagctggag ggctgcaccc
  121 aaagcctcag acctgctggg ggcccggggc ccagggggaa ccttccaggg ccgagatctt
  181 cgaggcgggg cccatgcctc ctcttcttcc ttgaacccca tgccaccatc gcagctgcag
  241 ctctcaacgg tggatgccca cgcccggacc cctgtgctgc aggtgcaccc cctggagagc
  301 ccagccatga tcagcctcac accacccacc accgccactg gggtcttctc cctcaaggcc
  361 cggcctggcc tcccacctgg gatcaacgtg gccagcctgg aatgggtgtc cagggagccg
  421 gcactgctct gcaccttccc aaatcccagt gcacccagga aggacagcac cctttcggct
  481 gtgccccaga gctcctaccc actgctggca aatggtgtct gcaagtggcc cggatgtgag
  541 aaggtcttcg aagagccaga ggacttcctc aagcactgcc aggcggacca tcttctggat
  601 gagaagggca gggcacaatg tctcctccag agagagatgg tacagtctct ggagcagcag
  661 ctggtgctgg agaaggagaa gctgagtgcc atgcaggccc acctggctgg gaaaatggca
  721 ctgaccaagg cttcatctgt ggcatcatcc gacaagggct cctgctgcat cgtagctgct
  781 ggcagccaag gccctgtcgt cccagcctgg tctggccccc gggaggcccc tgacagcctg
  841 tttgctgtcc ggaggcacct gtggggtagc catggaaaca gcacattccc agagttcctc
  901 cacaacatgg actacttcaa gttccacaac atgcgacccc ctttcaccta cgccacgctc
  961 atccgctggg ccatcctgga ggctccagag aagcagcgga cactcaatga gatctaccac
1021 tggttcacac gcatgtttgc cttcttcaga aaccatcctg ccacctggaa gaacgccatc
1081 cgccacaacc tgagtctgca caagtgcttt gtgcgggtgg agagcgagaa gggggctgtg
1141 tggaccgtgg atgagctgga gttccgcaag aaacggagcc agaggcccag caggtgttcc
1201 aaccctacac ctggcccctg acctcaagat caaggaaagg aggatggacg aacaggggcc
Human FOXP3 cDNA (GenBank Accession No.: BC113401):
SEQ ID NO: 19:
      1 atgcccaacc ccaggcctgg caagccctcg gccccttcct tggcccttgg cccatcccca
    61 ggagcctcgc ccagctggag ggctgcaccc aaagcctcag acctgctggg ggcccggggc
  121 ccagggggaa ccttccaggg ccgagatctt cgaggcgggg cccatgcctc ctcttcttcc
  181 ttgaacccca tgccaccatc gcagctgcag ctgcccacac tgcccctagt catggtggca
  241 ccctccgggg cacggctggg ccccttgccc cacttacagg cactcctcca ggacaggcca
  301 catttcatgc accagctctc aacggtggat gcccacgccc ggacccctgt gctgcaggtg
  361 caccccctgg agagcccagc catgatcagc ctcacaccac ccaccaccgc cactggggtc
  421 ttctccctca aggcccggcc tggcctccca cctgggatca acgtggccag cctggaatgg
  481 gtgtccaggg agccggcact gctctgcacc ttcccaaatc ccagtgcacc caggaaggac
  541 agcacccttt cggctgtgcc ccagagctcc tacccactgc tggcaaatgg tgtctgcaag
  601 tggcccggat gtgagaaggt cttcgaagag ccagaggact tcctcaagca ctgccaggcg
  661 gaccatcttc tggatgagaa gggcagggca caatgtctcc tccagagaga gatggtacag
  721 tctctggagc agcagctggt gctggagaag gagaagctga gtgccatgca ggcccacctg
  781 gctgggaaaa tggcactgac caaggcttca tctgtggcat catccgacaa gggctcctgc
  841 tgcatcgtag ctgctggcag ccaaggccct gtcgtcccag cctggtctgg cccccgggag
  901 gcccctgaca gcctgtttgc tgtccggagg cacctgtggg gtagccatgg aaacagcaca
  961 ttcccagagt tcctccacaa catggactac ttcaagttcc acaacatgcg accccctttc
1021 acctacgcca cgctcatccg ctgggccatc ctggaggctc cagagaagca gcggacactc
1081 aatgagatct accactggtt cacacgcatg tttgccttct tcagaaacca tcctgccacc
1141 tggaagaacg ccatccgcca caacctgagt ctgcacaagt gctttgtgcg ggtggagagc
1201 gagaaggggg ctgtgtggac cgtggatgag ctggagttcc gcaagaaacg gagccagagg
1261 cccagcaggt gttccaaccc tacacctggc ccctga
```

What is claimed is:

1. A method of treating a cardiovascular disease in a subject, comprising the step of administering to the subject a vector comprising a cDNA encoding a FOX (Forkhead box) protein, wherein the cDNA is under control of a disease-specific or constitutive promoter.

2. The method of claim 1, wherein the cardiovascular disease is atherosclerosis, coronary artery disease, or hyptertension.

3. The method of claim 1, wherein the FOX protein is FOXP3.

4. The method of claim 3, wherein the FOX protein is human FOXP3 (hFOXP3).

5. The method of claim 1, wherein the promoter is a disease-specific promoter.

6. The method of claim 5, wherein the promoter is a lectin-like oxidized low density lipoprotein receptor 1 (LOX-1) promoter.

7. The method of claim 1, wherein the promoter is a constitutive promoter.

8. The method of claim 7, wherein the promoter is a cytomegalovirus (CMV) immediate early promoter.

9. The method of claim 1, wherein the vector is an adeno-associated virus (AAV) vector.

10. The method of claim 9, wherein the vector comprises an AAV8 capsid gene.

11. The method of claim 10, wherein the AAV8 capsid gene comprises a nucleotide sequence selected from the group consisting of: (i) SEQ ID NO: 3; (ii) SEQ ID NO: 5; and (iii) SEQ ID NO: 7.


SEQUENCE LISTING (19 sequences)

SEQ ID NO: 1 - DNA, 2420 bp, Homo sapiens
atgaggcccacctacattatgcagcgaaatctactttcctctgctgattaatatgttaatctcatttaaaagcatcctgtctgggaaccccatgacccagtcaagaagacacagaaggccagcacggtggctcacggcttgtaatcccagcactgtgggaggctgaggcaggcggatcacttgaggacaggagttcaagaccagcctggccaacatggtgaaaccctgtctctactgaaaatacaaaacaattagccgggaggggtggcgggcacctgtaatcccagctactcaggaggctgaggcaggagaattgcttgaacccaggaggcggaggttgcagtgagccgagatcatgccattgcaacccaccctgggtaacaagggcgaaactccatctcaaaaacaaacaaacaaacaaacaaacaaacaaaacacatgaaattcaccatcacaggagttattgcttggtgggtatagagtttcagttggggaagatgaaaagttctggagatggatggtggtgatggctgcacaacaatgtaaatgtacttaatgccactgaacagtacgcctaaaaatggttgaaatggtaaacactttatgttctataatattcatggcatccaaaataactgagtcactgtagaaatgataatgtgcccttttcagtgtgatatcgtttcagtattttttaattctttttttttttttttttaaagggagtctcgctctgttgcccaggctggagtacagtggcacaatctcagctcactgaaaatgaaaagcattttcattttccacctcccaaatagctgggattataggcttgcaaccaggcccagctaacttttgtatttttagtggtgacagggtttcaacaggttggccaggctggtctcgaactcctgacctcagtgatccacccccccctcagcttcccaaagtgctgagattacaggcatgagccactgcacctggccaactttttcaatttgttctcaaatactctgcacataataaaaggaatataggaaacagggaggaggagggaaatggaatcctaattaggctgtcaatattggtaatttttcctaatagtttcctatatgttttatttatgtatttatttttccattcttcctttgtgtattcttttctttgcacttttttttcctggagtaatttaaagtaaatgccagatatcatatcattttgcttataaatacttccttcagaagacatctctaatagatatatatctggttaggaggttgtcaagggaaaatagaaaagtattctatcagtaaatacatatagacatacttatgggtctctcatgtaagcgtctttgtgacatgagtcaatcatggattttactgaaatacattatttgatgggtaattcaactgtgctgaatattagtactatactaataattattatttattggcttcttattatgctccaagtacaaagcttgtcatttttaaccagcgtgtcatttgaattcctatagcaacgtgacatttttcacaaacctagtgtaaaaaagcttattatttttacccagcatgtcatttgaattcctataacaatgttatcaggtagacattatccctaatttccagacatgtcatttgaattcctataacaatgttatcaggtagacattatccctaatttccagacaaagacatagattcaaagtattaagatttcccaagattgtagactaaaggaaagtactgaaactagctctttctgcattcaaagtgctctctgctgctacgttacactggcactgcatgaactagtagccccagataaattctaaaatcacccaggacacaccatcgctggaaatgggagccatatgtgattcaccccaaaattcttagatccattgccagataaaacagtgataatcaaaagagaatatatctgattcataaaagacaccatatagaaacagtctgctaatgaaattagcaaacaaacattggaatcaaatattttggcccataattctggcacattttttacaaatgtagtgtgacttactctctttgaatttcagtttctgtctctgaagagtgggtacaatatctcctctgatgctcatgaaaaatagttttccctttcataaattacttagcgaaatatcctgaaacaccttcagaatcaccactttctccacctgcaatacacataactcaagaatttgcgtcagcgaacttcccaatatgaagcaaagcctctccttcctcctaccaatgattgagccattcttctattagataacagtagctatttaaatacttctgcagaagctcacatatttttagtttgttgaagttcgtgactgcttcactctctcattcttagcttgaatttggaaatgacttttgatgacctaa

SEQ ID NO: 2 - DNA, 460 bp, Homo sapiens
aaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaag

SEQ ID NO: 3 - DNA, 2217 bp, Artificial Sequence
  Description of Artificial Sequence Synthetic polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 4 - protein, 738 aa, Artificial Sequence
  Description of Artificial Sequence Synthetic polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 5 - DNA, 2241 bp, Artificial Sequence
  Description of Artificial Sequence Synthetic polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagggacaacgagaataccaccactacaacaaagcacaagcagcacaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 6 - protein, 743 aa, Artificial Sequence
  Description of Artificial Sequence Synthetic polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQREYHHYNKAQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLT

SEQ ID NO: 7 - DNA, 2241 bp, Artificial Sequence
  Description of Artificial Sequence Synthetic polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagggacaacgagaataccaccactacaacaaagcacaagcagcacaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgtttcctcacccgtaatctgtaa

SEQ ID NO: 8 - protein, 743 aa, Artificial Sequence
  Description of Artificial Sequence Synthetic polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQGQREYHHYNKAQAAQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLT

SEQ ID NO: 9 - DNA, 34 bp, Artificial Sequence
  Description of Artificial Sequence Synthetic remains of pGL3 polylinker oligonucleotide
ggtaccgagctcttacgcgtgctagcccctgcag

SEQ ID NO: 10 - DNA, 145 bp, Artificial Sequence
  Description of Artificial Sequence Synthetic LT ITR (Inverted Terminal Repeat) polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 11 - DNA, 32 bp, Artificial Sequence
  Description of Artificial Sequence Synthetic oligonucleotide sequence between ITRs
gcggccgcatgcatagatctacgcgtctcgag

SEQ ID NO: 12 - DNA, 2972 bp, Artificial Sequence
  Description of Artificial Sequence Synthetic RT ITR (Inverted Terminal Repeat) plus remains of pGL3 polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactgcaggaattcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 13 - protein, 7 aa, Artificial Sequence
  Description of Artificial Sequence Synthetic 7-amino acid peptide that can be inserted into AAV capsid peptide
EYHHYNK

SEQ ID NO: 14 - DNA, 27 bp, Artificial Sequence
  Description of Artificial Sequence Synthetic upstream primer to generate Lox-1 promoter
atatgcatctttcttatttgggggaag

SEQ ID NO: 15 - DNA, 30 bp, Artificial Sequence
  Description of Artificial Sequence Synthetic downstream primer to generate Lox-1 promoter
atacgcgtactaaaaatatgtgagcttctg

SEQ ID NO: 16 - protein, 396 aa, Homo sapiens
MPNPRPGKPSAPSLALGPSPGASPSWRAAPKASDLLGARGPGGTFQGRDLRGGAHASSSSLNPMPPSQLQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 17 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 18 - DNA, 1260 bp, Homo sapiens
tcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggcc

SEQ ID NO: 19 - DNA, 1296 bp, Homo sapiens
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga